SEQUENCE LISTING (62 sequences)

SEQ ID NO: 1 - protein, 5 aa, synthetic construct
HHHHH

SEQ ID NO: 2 - protein, 4 aa, synthetic construct
AEEA

SEQ ID NO: 3 - protein, 7 aa, unidentified
PKKKRKV

SEQ ID NO: 4 - protein, 20 aa, unidentified
AVKRPAATKKAGQAKKKKLD

SEQ ID NO: 5 - protein, 9 aa, unidentified
PAAKRVKLD

SEQ ID NO: 6 - protein, 25 aa, unidentified
MSRRRKANPTKLSENAKKLAKEVEN

SEQ ID NO: 7 - protein, 9 aa, unidentified
KLKIKRPVK

SEQ ID NO: 8 - protein, 11 aa, unidentified
YGRJJRRQRRR

SEQ ID NO: 9 - protein, 16 aa, unidentified
MLGDFDLDMLGDFDLD

SEQ ID NO: 10 - protein, 10 aa, synthetic construct
KFFKFFKFFK

SEQ ID NO: 11 - DNA, 12 bp, synthetic construct
  source 1..12: mol_type = other DNA
caccggcaagtg

SEQ ID NO: 12 - protein, 9 aa, unidentified
ETFSDLWKL

SEQ ID NO: 13 - protein, 9 aa, unidentified
DDIEQWFTE

SEQ ID NO: 14 - protein, 9 aa, unidentified
SDIMDFVLK

SEQ ID NO: 15 - protein, 9 aa, unidentified
DLLDFSMMF

SEQ ID NO: 16 - protein, 9 aa, unidentified
ETLDFSLVT

SEQ ID NO: 17 - protein, 9 aa, unidentified
RKILNDLSS

SEQ ID NO: 18 - protein, 9 aa, unidentified
EAILAELKK

SEQ ID NO: 19 - protein, 9 aa, unidentified
DDVVQYLNS

SEQ ID NO: 20 - protein, 9 aa, unidentified
DDVYNYLFD

SEQ ID NO: 21 - protein, 9 aa, unidentified
DLFDYDFLV

SEQ ID NO: 22 - protein, 9 aa, unidentified
DFFDYDLLF

SEQ ID NO: 23 - protein, 9 aa, unidentified
EDLYSILWS

SEQ ID NO: 24 - protein, 9 aa, unidentified
TDLYHTLWN

SEQ ID NO: 25 - DNA, 17 bp, synthetic construct
  source 1..17: mol_type = other DNA
agtggtaccgtctgcta

SEQ ID NO: 26 - DNA, 17 bp, synthetic construct
  source 1..17: mol_type = other DNA
cttctactgctcacagg

SEQ ID NO: 27 - DNA, 17 bp, synthetic construct
  source 1..17: mol_type = other DNA
cgggtgcttgccatctt

SEQ ID NO: 28 - DNA, 17 bp, synthetic construct
  source 1..17: mol_type = other DNA
tgccattctgaagccgg

SEQ ID NO: 29 - DNA, 17 bp, Homo sapiens
  source 1..17: mol_type = genomic DNA
cgggtgcttgccatctt

SEQ ID NO: 30 - DNA, 17 bp, Homo sapiens
  source 1..17: mol_type = genomic DNA
gtgcttgccatcttcat

SEQ ID NO: 31 - DNA, 17 bp, Homo sapiens
  source 1..17: mol_type = genomic DNA
cttgccatcttcatctg

SEQ ID NO: 32 - DNA, 17 bp, Homo sapiens
  source 1..17: mol_type = genomic DNA
ccatcttcatctgcagc

SEQ ID NO: 33 - DNA, 17 bp, Homo sapiens
  source 1..17: mol_type = genomic DNA
cagcgggtgcttgccat

SEQ ID NO: 34 - DNA, 17 bp, Homo sapiens
  source 1..17: mol_type = genomic DNA
ttctgccattctgaagc

SEQ ID NO: 35 - DNA, 17 bp, Homo sapiens
  source 1..17: mol_type = genomic DNA
tgccattctgaagccgg

SEQ ID NO: 36 - DNA, 17 bp, Homo sapiens
  source 1..17: mol_type = genomic DNA
ccattctgaagccgggt

SEQ ID NO: 37 - DNA, 17 bp, Homo sapiens
  source 1..17: mol_type = genomic DNA
atcatcttctgccattc

SEQ ID NO: 38 - DNA, 17 bp, Homo sapiens
  source 1..17: mol_type = genomic DNA
atcttctgccattctga

SEQ ID NO: 39 - DNA, 17 bp, Mus musculus
  source 1..17: mol_type = genomic DNA
agtggtaccgtctgcta

SEQ ID NO: 40 - DNA, 17 bp, Mus musculus
  source 1..17: mol_type = genomic DNA
cttctactgctcacagg

SEQ ID NO: 41 - DNA, 15 bp, synthetic construct
  source 1..15: mol_type = other DNA
taaagttcgtttaga

SEQ ID NO: 42 - DNA, 15 bp, synthetic construct
  source 1..15: mol_type = other DNA
gctctccatcttacc

SEQ ID NO: 43 - DNA, 15 bp, synthetic construct
  source 1..15: mol_type = other DNA
acaccgcaaacccgt

SEQ ID NO: 44 - DNA, 15 bp, synthetic construct
  source 1..15: mol_type = other DNA
cgggctgcacttaca

SEQ ID NO: 45 - DNA, 15 bp, synthetic construct
  source 1..15: mol_type = other DNA
tactagtgcctgtgc

SEQ ID NO: 46 - DNA, 15 bp, synthetic construct
  source 1..15: mol_type = other DNA
gtatacgacatcagt

SEQ ID NO: 47 - protein, 22 aa, synthetic construct
AQQAQQQQQNQAQQAQQQQQNQ

SEQ ID NO: 48 - protein, 15 aa, synthetic construct
PPPDLGPPPDLGPPP

SEQ ID NO: 49 - protein, 19 aa, synthetic construct
KSHAHAQKRIRRRLLIILL

SEQ ID NO: 50 - DNA, 12 bp, synthetic construct
  source 1..12: mol_type = other DNA
atcaccaagtag

SEQ ID NO: 51 - DNA, 12 bp, synthetic construct
  source 1..12: mol_type = other DNA
tgtgttacgcta

SEQ ID NO: 52 - DNA, 12 bp, synthetic construct
  source 1..12: mol_type = other DNA
gtgacatacatt

SEQ ID NO: 53 - DNA, 17 bp, synthetic construct
  source 1..17: mol_type = other DNA
ccactgtcgagtcgcgt

SEQ ID NO: 54 - DNA, 21 bp, synthetic construct
  source 1..21: mol_type = other DNA
cgcagcgatatcgtcatccat

SEQ ID NO: 55 - DNA, 20 bp, synthetic construct
  source 1..20: mol_type = other DNA
cctgggggactttggaatca

SEQ ID NO: 56 - DNA, 20 bp, synthetic construct
  source 1..20: mol_type = other DNA
gacaacacgcggatgtgaga

SEQ ID NO: 57 - DNA, 19 bp, synthetic construct
  source 1..19: mol_type = other DNA
gcagcagcacatcaacaag

SEQ ID NO: 58 - DNA, 20 bp, synthetic construct
  source 1..20: mol_type = other DNA
cacgggaaagacacaggtag

SEQ ID NO: 59 - DNA, 20 bp, synthetic construct
  source 1..20: mol_type = other DNA
gtggaggcatgttcggtagt

SEQ ID NO: 60 - DNA, 20 bp, synthetic construct
  source 1..20: mol_type = other DNA
cctgcgttggatttcgtgac

SEQ ID NO: 61 - DNA, 21 bp, synthetic construct
  source 1..21: mol_type = other DNA
gttgtcaattgctgccctgtc

SEQ ID NO: 62 - DNA, 20 bp, synthetic construct
  source 1..20: mol_type = other DNA
cagtgacccctgatggatgt

(19) United States
(12) Patent Application Publication
(10) Pub. No.: US 2026/0232813 A1
(43) Pub. Date: Aug. 13, 2026

(54) COMPOSITION AND METHOD OF USE FOR A THERAPEUTIC NANOLIGOMER

(71) Applicant: Sachi Bioworks Inc., Louisville, CO (US)

(72) Inventor: Prashant Nagpal, Lafayette, CO (US)

(73) Assignee: Sachi Bioworks Inc., Louisville, CO (US)

(21) Appl. No.: 19/561,655

(22) Filed: Mar. 10, 2026

Related U.S. Application Data
(63) Continuation-in-part of application No. 18/079,312, filed on Dec. 12, 2022, now Pat. No. 12,636,303.
Application No. 18/079,312 is a continuation-in-part of application No. 17/460,968, filed on Aug. 30, 2021, now Pat. No. 11,530,406.

(51) Int. Cl. A61K 47/54; A61K 47/69

(52) U.S. Cl. CPC A61K 47/549; A61K 47/6929

(57) ABSTRACT

A therapeutic nanoligomer composition includes at least a peptide nucleic acid (PNA), the at least a PNA including a sequence of nucleobases capable of interacting with a gene target in a target host and a polypeptide backbone attached to the sequence of nucleobases, the polypeptide backbone including a plurality of amino acid units, wherein the plurality of amino acid units includes at least a 2-N-aminoethylglycine unit; and a transport domain, the transport domain including a delivery nanoparticle and a cellular uptake domain (CUD) associated with the delivery nanoparticle, wherein the delivery nanoparticle is attached to the at least a PNA, and wherein the CUD includes a hydrophilic polymeric coating or one or more weakly charged surface groups.

### CROSS-REFERENCE TO RELATED APPLICATIONS

This application is a continuation-in-part of U.S. Non-provisional application Ser. No. 18/079,312 filed on Dec. 12, 2022, and entitled “COMPOSITION OF A THERAPEUTIC OLIGOMER”, which is a continuation-in-part of U.S. Non-provisional application Ser. No. 17/460,968 filed on Aug. 30, 2021, now U.S. Pat. No. 11,530,406 issued on Dec. 20, 2022, and entitled “SYSTEM AND METHOD FOR PRODUCING A THERAPEUTIC OLIGOMER”, the entirety of each of which is incorporated herein by reference.

### FIELD OF THE INVENTION

The present invention generally relates to the field of DNA therapeutics. In particular, the present invention is directed to compositions and methods of use for a therapeutic nanoligomer.

### REFERENCE TO SEQUENCE LISTING

This specification includes a sequence listing submitted herewith, which includes the file entitled 1186-001USC2.xml having the following size: 54,872 bytes which was created Jan. 26, 2026, the contents of which are incorporated by reference herein.

### BACKGROUND

An encroaching crisis of limited pharmaceutical treatment options is rising exponentially. This is further complicated by the dwindling pipeline of treatment options for many healthcare challenges.

### SUMMARY OF THE DISCLOSURE

In an aspect, a therapeutic nanoligomer composition includes at least a peptide nucleic acid (PNA), the at least a PNA including a sequence of nucleobases capable of interacting with a gene target in a target host and a polypeptide backbone attached to the sequence of nucleobases, the polypeptide backbone including a plurality of amino acid units, wherein the plurality of amino acid units includes at least a 2-N-aminoethylglycine unit; and a transport domain, the transport domain including a delivery nanoparticle and a cellular uptake domain (CUD) associated with the delivery nanoparticle, wherein the delivery nanoparticle is attached to the at least a PNA, and wherein the CUD includes a hydrophilic polymeric coating or one or more weakly charged surface groups.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for producing a therapeutic oligomer;

FIG. 2 is a block diagram illustrating an exemplary embodiment of a criterion element;

FIGS. 3A-B are diagrammatic representations illustrating exemplary embodiments of a PNA;

FIG. 4 is a diagrammatic representation illustrating an exemplary embodiment of a peptide synthesis;

FIG. 5 is a block diagram of an exemplary embodiment of a neural network;

FIG. 6 is a block diagram of an exemplary embodiment of a node in a neural network;

FIG. 7 is a block diagram of an exemplary embodiment of a machine-learning module;

FIG. 8 is a block diagram of an exemplary embodiment of a modular approach;

FIG. 9 is a diagrammatic representation of a regulation modification;

FIGS. 10A-C are diagrammatic representations of exemplary embodiments of a therapeutic effect;

FIG. 11 is a block diagram illustrating an exemplary embodiment of a proposed therapeutic oligomer;

FIGS. 12A-C are diagrammatic representations illustrating exemplary embodiments of a therapeutic oligomer/nanoligomer;

FIGS. 13A-C are diagrammatic representations illustrating exemplary embodiments of a therapeutic effect of a nanoligomer;

FIGS. 14A-C are diagrammatic representations illustrating exemplary embodiments of a therapeutic effect of an antiviral nanoligomer;

FIGS. 15A-C are diagrammatic representations illustrating exemplary embodiments of an in vivo efficacy assessment of therapeutic oligomer for treating SARS-COV-2;

FIG. 16 is a diagrammatic representation illustrating an exemplary embodiment of a therapeutic effect of a nanoligomer;

FIGS. 17A-B are diagrammatic representations illustrating exemplary embodiments of a toxicity of a therapeutic oligomer;

FIGS. 18A-C are diagrammatic representations illustrating exemplary embodiments of an efficacy of a therapeutic oligomer;

FIG. 19 is a diagrammatic representation illustrating an exemplary embodiment of a therapeutic effect of a nanoligomer;

FIGS. 20A-B are diagrammatic representations illustrating exemplary embodiments of a therapeutic effect of a nanoligomer;

FIGS. 21A-B are diagrammatic representations illustrating exemplary embodiments of a genomic outcome;

FIGS. 22A-B are diagrammatic representations illustrating exemplary embodiments of a therapeutic effect of a nanoligomer;

FIGS. 23A-C are diagrammatic representations illustrating exemplary embodiments of a therapeutic effect of a nanoligomer;

FIG. 24 is a block diagram of an exemplary embodiment of a naive Bayes classifier;

FIG. 25 is a flow diagram illustrating a method for producing a therapeutic nanoligomer; and

FIG. 26 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to a composition of a therapeutic oligomer. Therapeutic nanoligomer composition includes at least a peptide nucleic acid (PNA) and a transport domain. At least a PNA includes a sequence of nucleobases capable of interacting with a gene target in a target host and a polypeptide backbone attached to the sequence of nucleobases, wherein the polypeptide backbone includes a plurality of amino acid units. Transport domain includes a delivery nanoparticle, wherein the delivery nanoparticle is attached to at least a PNA.

Aspects of the present disclosure may be used to provide means of modulating gene expression using peptide nucleic acid (PNA) nanoligomers as therapeutic agents. Aspects of the present disclosure may be used to improve the specificity and efficacy of antisense-based therapies. Exemplary embodiments illustrating aspects of the present disclosure are described below in the context of several specific therapeutic indications and experimental models.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is understood that the acts described below are meant as a general overview and demonstration of an exemplary method, and that the method may include different and/or additional acts as described herein or otherwise.

While the present invention will be described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (for example, a step is performed by or with the assistance of a human).

For the purposes of this disclosure, including the claims, the phrase “at least some” means “one or more” and includes the case of only one. Thus, for example, the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, for example, the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, for example, the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, for example, the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (for example, “(A)”, “(B)”, “(C)”, and so on, or “(a)”, “(b)”, and so on) and/or numbers (for example, “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, for example, the phrase “multiple ABCs” means “two or more ABCs” and includes “two ABCs”. Similarly, for example, the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, for example, “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (for example, by reducing one or more symptoms of a neurological disorder). The term “therapeutic” includes the effect of mitigating deleterious clinical effects of certain processes (i.e., consequences of the process, rather than the symptoms of processes).

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and embodiments thereof are further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

### Definitions

Definitions of specific functional groups and chemical terms are described in more detail below. For purposes of this invention, the chemical elements are identified in accordance with the Periodic Table of the Elements, Chemical Abstracts Service (CAS) version of periodic table of the elements, Handbook of Chemistry and Physics, 106th Ed., inside cover, and specific functional groups are generally defined as described therein. Additionally, general principles of organic chemistry, as well as specific functional moieties and reactivity, are described in Organic Chemistry, Thomas Sorrell, 2nd Edition, University Science Books, 2006; Smith, March's Advanced Organic Chemistry: Reactions, Mechanisms, and Structure, 9th Edition, Wiley, 2025; Larock, Comprehensive Organic Transformations, 3rd Edition, Wiley, 2018; Carruthers and Coldham, Modern Methods of Organic Synthesis, 4th Edition, Cambridge University Press, Cambridge, 2004.

“Amino acid,” as used in this disclosure, includes any naturally occurring and non-naturally occurring amino acid, including without limitation natural alpha-amino acids such as the 20 common naturally occurring alpha-amino acids found in polypeptides and proteins (A, R, N, D, C, Q, E, G, H, I, L, K, M, F, P, S, T, W, Y, V, also referred to as standard amino acids), non-standard alpha-amino acids, and beta-amino acids, and there are many known non-standard (e.g., non-natural) amino acids, any of which may be included in the polypeptides or proteins described herein. See, for example, S. Hunt, The Non-Protein Amino Acids in Chemistry and Biochemistry of the Amino Acids, edited by G. C. Barrett, Chapman and Hall, 1985, and/or Hughes, B. (ed.), Amino Acids, Peptides and Proteins in Organic Chemistry, Volumes 1-4, Wiley-VCH (2009-2011); Blaskovich, M., Handbook on Syntheses of Amino Acids: General Routes to Amino Acids, Oxford University Press, 2010.

“Protein,” “peptide,” and “polypeptide” are terms used interchangeably herein and refer to a polymer of amino acid residues linked together by peptide (amide) bonds. A protein, peptide, or polypeptide may be of any size, structure, or function and may be at least three amino acids long. In some cases, the term may refer to an individual protein, and in other cases, it may refer to a collection of proteins. A protein, peptide, or polypeptide may be modified by one or more chemical entities. In some cases, such modifications may include the addition of a carbohydrate group, a hydroxyl group, a phosphate group, a farnesyl group, an isofarnesyl group, or a fatty acid group. In some cases, a modification may include addition of a linker for conjugation, functionalization, or other chemical modification. A protein, peptide, or polypeptide may be a single molecule or a multi-molecular complex, and it may also be a fragment of a naturally occurring protein or peptide. In some cases, the protein, peptide, or polypeptide may be naturally occurring, recombinant, synthetic, or any combination thereof. In some cases, a peptide may be between 3 and 60 amino acids long, for example between 3 and 15 amino acids, between 15 and 30 amino acids, between 30 and 45 amino acids, or between 45 and 60 amino acids.

“Genetically engineered,” “genetically modified,” or “recombinant,” are terms encompass nucleic acids whose sequence comprises a non-naturally occurring sequence. A non-naturally occurring sequence is one invented or generated by man and not occurring in nature or not known to occur in nature. These terms also encompass nucleic acids comprising two or more naturally occurring sequences joined together, where such sequences are not found joined to one another in their naturally occurring state. Further, these terms include nucleic acids comprising a deletion, insertion, rearrangement, or other alteration of or within a naturally occurring sequence, wherein such deletion, insertion, rearrangement, or other alteration is brought about by the hand of man. The terms “genetically engineered,” “genetically modified,” or “recombinant” polypeptide encompass polypeptides encoded by genetically engineered nucleic acids. In some embodiments, the sequence of a genetically engineered polypeptide expressed by a cell is distinct from those polypeptides that are endogenous to the cell. The terms “genetically engineered cell,” “genetically modified cell,” or “recombinant cell” encompass cells into which a nucleic acid has been introduced by the hand of man, as well as their descendants that inherit at least a portion of the introduced nucleic acid. In some embodiments, a genetically engineered cell has had its genome altered by the hand of man, for example by insertion of an exogenous nucleic acid sequence and/or deletion of an endogenous nucleic acid sequence. A genetically engineered cell may also be a descendant of such a cell and may inherit a copy of at least a portion of the original alteration. In some embodiments, the nucleic acid, or a portion thereof, or a copy of the nucleic acid or a portion thereof, may be integrated into the genome of the cell. The terms “non-genetically engineered,” “non-genetically modified,” and “non-recombinant” refer to the absence of genetic engineering or genetic modification. Non-genetically engineered polypeptides encompass endogenous polypeptides. In certain embodiments, a non-genetically engineered cell, gene, or genome does not contain non-endogenous nucleic acid, such as DNA or RNA that originates from a vector, from a different species, or that comprises an artificial sequence, for example DNA or RNA introduced by the hand of man. In certain embodiments, a non-genetically engineered cell has not been intentionally contacted with a nucleic acid that is capable of causing a heritable genetic alteration under conditions suitable for uptake of the nucleic acid by the cells.

A “vector” may be any of a number of nucleic acid molecules, viruses, or portions thereof that are capable of mediating entry of, for example, transferring, transporting, or otherwise delivering, a nucleic acid of interest between different genetic environments or into a cell. The nucleic acid of interest may be linked to, or inserted into, the vector using, for example, restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, or transposons (for example, the Sleeping Beauty transposon). Plasmid vectors typically include an origin of replication, for example, replication in prokaryotic cells. A plasmid may include part or all of a viral genome, for example, a viral promoter, enhancer, processing or packaging signals, or sequences sufficient to give rise to a nucleic acid that can be integrated into the host cell genome and/or to give rise to infectious virus. Viruses or portions thereof that can be used to introduce nucleic acids into cells may be referred to as viral vectors. Viral vectors include, for example, adenoviruses, adeno-associated viruses, retroviruses (such as lentiviruses or gamma retroviruses), vaccinia virus and other poxviruses, herpesviruses (such as herpes simplex virus), and others. Viral vectors may or may not contain sufficient viral genetic information for production of infectious virus when introduced into host cells, i.e., viral vectors may be replication-competent or replication-defective. In some embodiments, for example where sufficient information for production of infectious virus is lacking, it may be supplied by a host cell or by another vector introduced into the cell if production of virus is desired. In other embodiments, such information is not supplied if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, proteins that increase or decrease resistance or sensitivity to antibiotics or other agents (such as a protein conferring resistance to puromycin, hygromycin, or blasticidin), enzymes whose activities are detectable by assays known in the art (for example, β-galactosidase or alkaline phosphatase), and proteins or RNAs that detectably affect the phenotype of cells that express them (for example, fluorescent proteins). Vectors often include one or more appropriately positioned sites for restriction enzymes, which may be used to facilitate insertion into the vector of a nucleic acid, for example a nucleic acid to be expressed. An expression vector is a vector into which a desired nucleic acid has been inserted or may be inserted such that it is operably linked to regulatory elements (also termed “regulatory sequences,” “expression control elements,” or “expression control sequences”) and may be expressed as an RNA transcript, for example an mRNA that can be translated into protein or a noncoding RNA such as an shRNA or miRNA precursor. Expression vectors include regulatory sequence(s), for example expression control sequences, sufficient to direct transcription of an operably linked nucleic acid under at least some conditions; other elements required or helpful for expression may be supplied by, for example, the host cell or by an in vitro expression system. Such regulatory sequences typically include a promoter and may include enhancer sequences or upstream activator sequences. In some embodiments, a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired, may be contained in an inserted nucleic acid, or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species, for example a mammalian species, or in a desired cell type. A vector may contain a promoter capable of directing expression in mammalian cells, such as a suitable viral promoter, for example from cytomegalovirus (CMV), retrovirus, simian virus (for example SV40), papillomavirus, herpesvirus or other virus that infects mammalian cells, or a mammalian promoter from, for example, a gene such as EF1alpha, ubiquitin (such as ubiquitin B or C), globin, actin, phosphoglycerate kinase (PGK), or a composite promoter such as a CAG promoter (combination of the CMV early enhancer element and chicken beta-actin promoter). In some embodiments, a human promoter may be used. In some embodiments, a promoter that ordinarily directs transcription by a eukaryotic RNA polymerase I (a “pol I promoter”), for example a promoter for transcription of ribosomal RNA (other than 5S rRNA) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III (a “pol III promoter”), for example a promoter for transcription of U6, H1, 7SK or tRNA or a functional variant thereof, is used. One of ordinary skill in the art will select an appropriate promoter for directing transcription of a sequence of interest. Examples of expression vectors that may be used in mammalian cells include, for example, the pcDNA vector series, pSV2 vector series, pCMV vector series, pRSV vector series, pEF1 vector series, and Gateway® vectors. Examples of virus vectors that may be used in mammalian cells include, for example, adenoviruses, adeno-associated viruses, poxviruses such as vaccinia viruses and attenuated poxviruses, retroviruses (for example lentiviruses), Semliki Forest virus, Sindbis virus, and others. In some embodiments, regulatable (for example, inducible or repressible) expression control element(s), for example a regulatable promoter, is/are used so that expression can be regulated, for example turned on or increased or turned off or decreased. For example, the tetracycline-regulatable gene expression system (Gossen & Bujard, Proc. Natl. Acad. Sci. 89:5547-5551, 1992) or variants thereof (see, for example, Allen, N, et al. (2000), Urlinger, S, et al. (2000), Zhou, X., et al (2006)) can be employed to provide inducible or repressible expression. Other inducible/repressible systems may be used in various embodiments. For example, expression control elements that can be regulated by small molecules such as artificial or naturally occurring hormone receptor ligands (for example steroid receptor ligands such as naturally occurring or synthetic estrogen receptor or glucocorticoid receptor ligands), tetracycline or analogs thereof, or metal-regulated systems (for example the metallothionein promoter) may be used in certain embodiments. In some embodiments, tissue-specific or cell type-specific regulatory element(s) may be used, for example to direct expression in one or more selected tissues or cell types.

In some embodiments, a vector is used to insert exogenous DNA into the genome of a cell. In general, any suitable vector may be used. In some embodiments, the vector is a viral vector, for example, a retroviral vector such as a lentiviral vector or gamma retroviral vector, or an adenoviral or adeno-associated viral (AAV) vector. In some embodiments, the vector is a plasmid, for example, a DNA plasmid. In some embodiments, the plasmid comprises DNA to be inserted into the genome of a cell, wherein the DNA is located between binding sites for a transposase (“transposase binding sites”) so that integration of the DNA can be achieved by supplying the transposase, for example, by expressing it from the same or a different plasmid. In some embodiments, the transposase is, for example, a member of the Sleeping Beauty family of transposases, the piggyBac family of transposases, or the Tol2 family of transposases (see Grabundzija, I., et al., Molecular Therapy, vol. 18 no. 6, 1200-1209 (2010) for review of transposon systems that utilize these transposases, and various uses thereof in genetic engineering). Examples of Sleeping Beauty transposases include SB10, SB11, and SB100X (see, for example, Mates, L., et al., Nat Genet. (2009) 41 (6): 753-61; Jin, Z., et al., Gene Therapy (2011) 18, 849-856). In some embodiments, the vector is suitable for use to genetically engineer cells, for example, human cells, which are to be administered to a human subject. In some embodiments, the vector has been used in at least one clinical trial in human subjects, results of which have been published, without reported clinically unacceptable adverse events attributable to the vector. In some embodiments, the vector is a self-inactivating retroviral vector. Such vectors may be created by deletion of at least part of the U3 portion of the 3′ LTR. Exemplary retroviral and lentiviral vectors are described in US Pat. Pub. No. 20050251872, US Pat. Pub. No. 20040259208, and various other references cited herein. In some embodiments, a second- or third-generation lentiviral vector may be used.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while intermediate or low stringency hybridization can be used to identify or detect polynucleotide sequence homologs.

For the purpose of this disclosure, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

For the purpose of this disclosure, in one embodiment, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double-stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing, and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

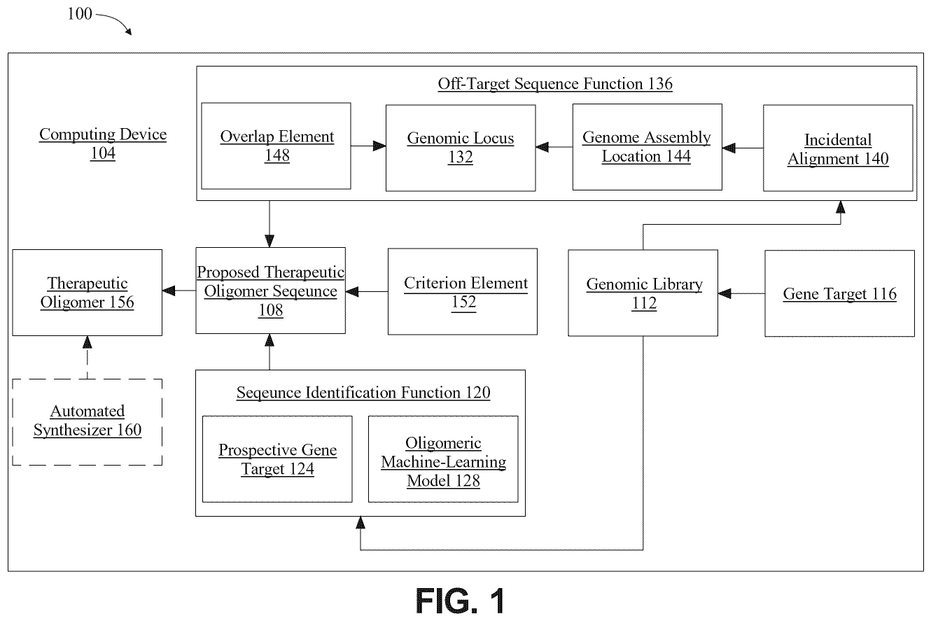

Referring now to FIG. 1, an exemplary embodiment of a system 100 for producing a therapeutic oligomer is illustrated. System 100 includes a computing device 104. Computing device 104 may include any computing device as described in this disclosure, including, without limitation, a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing devices operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, a “therapeutic oligomer”, as used herein, is a polymer comprising relatively few repeating units that may produce a therapeutic effect as a function of regulating an expression of one or more genes and/or polynucleotides. As used in this disclosure, a “therapeutic effect” is a response of an organism that occurs as a function of an external stimulus, such as but not limited to an oligomer. In an embodiment, and without limitation, an organism may include one or more archaea, bacteria, eukarya, and the like thereof. For example, and without limitation, organisms may include one or more prokaryota cells, halophiles, hyperthermophiles, and the like thereof. As a further nonlimiting example, organisms may include one or more bacteria. As a further nonlimiting example, organisms may include one or more humans, pets, animals, and the like thereof. As used in this disclosure, a “polynucleotide”, is a polymer of nucleic acid residues of any length. In an embodiment, polynucleotide May contain deoxyribonucleotides, ribonucleotides, and/or their analogs and may be double-stranded or single-stranded. Polynucleotide may include natural nucleic acids, modified nucleic acids (e.g., methylated), nucleic acid analogs, and/or non-naturally occurring nucleic acids, and may be interrupted by non-nucleic acid residues. For example, and without limitation, polynucleotide may include a gene, a gene fragment, cDNA, isolated DNA, mRNA, tRNA, rRNA, isolated RNA of any sequence, recombinant polynucleotides, primers, probes, plasmids, vectors, and the like thereof. In an embodiment, and without limitation, polynucleotide may include nucleic acid polymers that have been modified, whether naturally or by intervention.

With continued reference to FIG. 1, for the purposes of this disclosure, an “oligomer” is a chemical structure that contains a limited number of repeating monomer units, typically ranging from two to a few dozen. Unlike polymers, which may consist of thousands of monomer units, oligomers have a shorter chain length, conferring distinct physical and chemical properties. Oligomers may include various monomer types, including nucleotides, amino acids, or other organic molecules, and may be linear, cyclic, or branched in structure. They are utilized in various industrial and pharmaceutical applications due to their unique characteristics, such as ease of synthesis, tunable properties, and specific interactions with biological targets. In some cases, therapeutic oligomer may include a therapeutic nanoligomer. For the purposes of this disclosure, a “nanoligomer” is a small, synthetic oligomeric molecule designed to interact specifically with a target nucleic acid or protein for medical applications. Nanoligomer may include a size in the range of 0.1 nm to 100 nm. Nanoligomers are typically less than 10 nanometers in size and are often used to silence genes, modulate gene expression, inhibit protein interactions, or correct genetic mutations. Applications of oligomers may include treating genetic disorders, cancers, and/or infectious diseases. Nonlimiting examples of nanoligomers may include antisense oligonucleotides and small interfering RNA (siRNA). In one or more embodiments, nanoligomer may include a peptide nucleic acid (PNA) oligonucleotide that is complementary to a selected DNA or RNA sequence. In one or more embodiments, PNA may be conjugated to a nanoparticle to enable delivery. In one or more embodiments, PNA may form Watson-Crick duplexes with nucleic acids. In one or more embodiments, nanoligomers may include nanoscale peptide nucleic acid (PNA)-based constructs, typically less than 10 nanometers in effective dimension, which may be configured to modulate biological function through sequence-specific binding to nucleic acid targets. A nanoligomer may include a PNA oligomer having a defined nucleotide length selected to be complementary to a target DNA or RNA sequence, and may further include a carrier, conjugate, or nanoparticle to facilitate cellular uptake and intracellular localization. Due to a neutral backbone and high binding affinity of PNA, nanoligomers form stable and sequence-specific complexes with nucleic acids through Watson-Crick base pairing, while resisting enzymatic degradation. In contrast to nuclease-dependent antisense agents, nanoligomers may function through steric and structural interference, rather than cleavage of the target nucleic acid. In one or more embodiments, nanoligomers may be configured to silence or down-regulate gene expression at a post-transcriptional level by binding to messenger RNA at or near translation-critical regions, including start codons, untranslated regions, internal ribosome entry sites, or other regulatory motifs. Binding of a nanoligomer to a target RNA may physically block ribosome assembly, ribosome scanning, or translation initiation, thereby reducing synthesis of an encoded protein without degrading RNA transcript. In one or more embodiments, nanoligomers may additionally modulate transcription by binding to genomic DNA within promoters, enhancers, transcription start sites, or transcription factor binding motifs. Such binding may alter local DNA structure or accessibility, thereby inhibiting or facilitating recruitment of transcriptional machinery and resulting in down-regulation or up-regulation of gene expression. In one or more embodiments, nanoligomers may be used to inhibit protein-nucleic acid interactions by occupying binding sites on RNA or DNA that may be normally recognized by transcription factors, RNA-binding proteins, splicing factors, or other regulatory proteins. By forming stable PNA-nucleic acid complexes, Nanoligomer may prevent association of these proteins with their native binding motifs, thereby altering downstream processes such as transcription, RNA stability, translation efficiency, or alternative splicing. In one or more embodiments, nanoligomers may be configured to modulate pre-mRNA splicing by binding to splice sites or splicing regulatory elements, resulting in exon skipping, exon inclusion, or altered isoform expression. In one or more embodiments, nanoligomers may be configured to correct or compensate for genetic mutations through allele-specific silencing, splice redirection, or functional suppression of mutant gene products. Due to high mismatch discrimination, a nanoligomer may preferentially bind a mutant nucleic acid sequence while exhibiting reduced binding to a corresponding wild-type sequence, thereby selectively inhibiting expression of a pathogenic allele. In one or more embodiments, nanoligomers may further compensate for mutations by promoting production of functional or less deleterious protein isoforms through splicing modulation. Nanoligomers may offer high specificity and potency, effectively targeting molecular pathways at low concentrations, making them versatile and promising therapeutic agents. In some cases, “oligomer” and “nanoligomer” may be used interchangeably in this disclosure. In some cases, accordingly, “therapeutic oligomer” and “therapeutic nanoligomer” may be used interchangeably in this disclosure.

In an embodiment, and still referring to FIG. 1, proposed therapeutic oligomer sequence 108 may include a proposed peptide nucleic acid (PNA). As used in this disclosure, a “peptide nucleic acid” is a synthetic DNA-analog with a backbone of peptide units, as opposed to a phosphate backbone, that is linked to a plurality of nucleobases. As a nonlimiting example, peptide unit may include a 2-N-aminoethylglycine unit with its amino nitrogen bonded to a nucleobase via a COCH₂ linker. In one or more embodiments, pseudopeptide backbone may include a plurality of 2-N-aminoethylglycine units (also referred to as N-(2-aminoethyl)glycine), each unit bearing a nucleobase (e.g., adenine, cytosine, guanine, thymine, or uracil) through a COCH₂ (methylene carbonyl) linker attached to a backbone nitrogen. In one or more embodiments, PNAs may be electrically neutral over physiological pH, yet retain an ability to form sequence-specific Watson-Crick base pairs with complementary DNA or RNA targets. In one or more embodiments, PNA may form sequence-specific base pairs with higher binding affinity and thermal stability than a corresponding DNA or RNA oligomer of a same sequence. In one or more embodiments, PNA may exhibit increased resistance to degradation by nucleases and proteases relative to natural nucleic acids, thereby providing enhanced stability in biological fluids and within cells. For the purposes of this disclosure, a “linker” is a chemical structure that connects two or more elements together while keeping them separated by a certain distance. A linker usually connects to its targets via chemical bonds, such as covalent bonds. A linker typically contains non-reactive chemical structures such as aliphatic chains including methylene groups, aromatic chains including phenylene groups, polar chains including peptide groups and ethylene oxide groups, or the like. Additional details regarding linkers will be provided below in this disclosure. In an embodiment, and without limitation, proposed PNA may include a backbone composed of peptide bonds linking nucleobases. In another embodiment, and without limitation, proposed PNA may include an amino-terminal and/or a carboxy-terminal end. In another embodiment, and without limitation, proposed PNA may include a 5′ and/or a 3′ end in a conventional sense, with reference to a complementary nucleic acid sequence to which it specifically hybridizes. In an embodiment, proposed PNA may include a sequence that may be described in a conventional fashion similar to DNA and/or RNA, such as but not limited to having nucleobases including guanine (G), uracil (U), thymine (T), adenine (A), and/or cytosine (C) which may correspond to a nucleotide sequence of a DNA molecule. In an embodiment, proposed PNA may be synthesized using an automated DNA synthesizer, as described below in detail. In an embodiment, proposed PNA may be resistant to proteases and/or nucleases as a function of a structural difference from DNA, wherein the structure difference may result in the proposed PNA not being recognized by a hepatic transporter(s) recognizing DNA. In another embodiment, proposed PNA may comprise at least one modified phosphate backbone such as, but not limited to phosphorothioate, phosphorodithioate, 5-phosphoramidothioate, phosphoramidate, phosphordiamidate, methylphosphonate, alkyl phosphotriester, formacetal, and/or the like thereof. Additionally, or alternatively, proposed therapeutic oligomer sequence 108 may include an antisense oligonucleotide. As used in this disclosure, an “antisense oligonucleotide” is an antisense molecule that modulates the expression of one or more genes and/or polynucleotides. For example, and without limitation, antisense oligonucleotide may include antisense PNAs, antisense RNAs, and the like thereof. In another embodiment, antisense oligonucleotides may include RNA and/or DNA oligomers such as but are not limited to interfering RNA molecules, such as dsRNA, dsDNA, mRNA, siRNA, and/or hpRNA, as well as locked nucleic acids, bridged nucleic acid (BNA), polypeptides, and/or other oligomers and the like thereof.

With continued reference to FIG. 1, therapeutic oligomer sequence 108, therapeutic oligomer, and/or therapeutic nanoligomer may include a nucleic acid-binding domain. Such nucleic acid-binding domain may include or be included in a peptide nucleic acid (PNA), consistent with details described above. Such nucleic acid-binding domain may be configured to bind to a gene target or DNA, as described in detail below, to activate or repress its transcription on demand. In some cases, such binding may be achieved via complementary base pairs. “Nucleic acid-binding domain,” as used in this disclosure, is a region that recognizes and binds to nucleic acids. Nucleic acids may include DNA or RNA. In one or more embodiments, nucleic acid-binding domain may include sequence-defined PNA region that hybridizes to an target nucleic acid. In one or more embodiments, nucleic-acid domain may recognize a complementary sequence on a target mRNA or DNA via standard Watson-Crick base pairing. In one or more embodiments, region may be bound by nucleic-acid domain may include start codon, untranslated regions (UTR), promoter regions. In one or more embodiments, nucleic-acid binding domain may create a physical block that drives a drug effect. Nucleic-acid binding domain may hybridize near a AUG start codon or regulatory UTR sites. In one or more embodiments, nucleic-acid binding domain may be configured to sterically block ribosome assembly/initiator complex at a start codon. In one or more embodiments, nucleic-acid binding domain may sterically block RNA-binding proteins/structural element that may be required for efficient translation or stability. In one or more embodiments, nucleic-acid binding domain may result in reduced translation of an encoded protein without necessarily degrading a transcript of the protein. In one or more embodiments, nucleic-acid binding domain may be directed to genomic DNA motifs. For example and without limitation, genomic DNA motifs may include promoter or regulatory regions. Nucleic-acid binding domain interacting with genomic DNA motifs may alter transcription factor access or local structure, thereby modulating transcription through up-regulation or down-regulation. In one or more embodiments, nucleic-acid binding domain may be an effector, where it may act as a part of a molecule whose physical presence on a target nucleic acid causes an intended inhibition or modulation. In some embodiments, a PNA may include a synthetic DNA-analog where a phosphodiester bond is replaced with amino acid units, such as without limitation 2-N-aminoethylglycine units; in other words, a PNA may include a polypeptide backbone. As used in the current disclosure, a “synthetic DNA-analog” is a man-made compound which is analogous structurally to naturally occurring RNA and DNA. Nucleic acids are chains of nucleotides, which are composed of three parts: a phosphate backbone, a pentose sugar, either ribose or deoxyribose, and one of four nucleobases. A synthetic DNA-analog may have one or more of these parts. In some embodiments, the synthetic DNA-analogs nucleobases confer, among other things, different base pairing and base stacking properties. Examples include universal bases, which can pair with all four canonical bases, and phosphate-sugar backbone analogues such as PNA, which affect the properties of the chain (PNA can even form a triple helix). Artificial nucleic acids include peptide nucleic acid (PNA), Morpholino and locked nucleic acid (LNA), as well as glycol nucleic acid (GNA), threose nucleic acid (TNA), and hexitol nucleic acids (HNA). Each of these is distinguished from naturally occurring DNA or RNA by changes to the backbone of the molecule. As used in the current disclosure, a “phosphodiester bond” is a linkage that results from exactly two of the hydroxyl groups (—OH) in phosphoric acid reacting with two hydroxyl groups on other molecules to form two ester bonds, wherein the bonds make up the backbones of DNA and RNA. A phosphodiester bond includes a C—O—PO₂O—C linkage. As a result, a phosphate group is attached to the 5′ carbon of a sugar molecule. The 3′ carbon of one sugar molecule is bonded to the 5′ phosphate of an adjacent sugar molecule. Specifically, the phosphodiester bond links the 3′ carbon atom of one sugar molecule and the 5′ carbon atom of another. Such sugar molecule may include pentose such as deoxyribose in DNA or ribose in RNA. Due to the small pKa of the remaining-OH group, phosphodiesters are negatively charged at pH 7. Repulsion between these negative charges may influence the conformation of a nucleic acid. The negative charge may attract histones, metal cations such as magnesium, and polyamines. PNA may include 2-N-aminoethylglycin. In some embodiments, 2-N-aminoethylglycin may be identified by its molecular formula of C₄H₁₀N₂O₂.

Further referring to FIG. 1, therapeutic oligomer sequence 108, therapeutic oligomer, and/or therapeutic nanoligomer may include a nanoparticle binding element. A “nanoparticle binding element”, as used in this disclosure, is a chemical structure attaches a chemical agent with a therapeutic effect to a nanoparticle, as described in further detail below. A nanoparticle binding element may include a functional group, a chemical moiety, a molecule, a monomer, an oligomer, and/or a polymer. A nanoparticle binding element may include a first end attached to therapeutic oligomer sequence 108, therapeutic oligomer, and/or therapeutic nanoligomer, where the attachment may be performed using any form of chemical, covalent, and/or ionic bond, including bonds effected using a polymerase. Nanoparticle binding element may include a second end, which may be an opposite end of the nanoparticle binding element from the first end. Nanoparticle binding element may function, without limitation, as a nanoparticle-binding domain (NBD), which may include a peptide or other sequence added to combine with therapeutic oligomer sequence 108, therapeutic elements, and/or sequences for conjugation and/or attachment with a nanoparticle. In an embodiment, this may allow for rapid and/or low-cost and scalable purification of full-sequence peptide-backbone-based molecules, while attaching desired elements to form a nanoligomer for active transport. Typical NBD sequences used with gold nanoparticles may include a histidine tag such as HHHHH (5-histidine, SEQ ID NO: 1), cysteine, or the like. Additional nonlimiting examples of NBD sequences may include amine, pyridine, imidazole, thiols/thiolates, dithiols/dithiolates, carboxylic acids/carboxylates, phosphonic acids/phosphonates, among other functional groups recognized by a person of ordinary skill in the art, upon reviewing the entirety of this disclosure. Once attached to a nanoparticle, such sequences and/or elements may also allow selective attachment and potential folding of peptide and PNA-based sequences, to keep low hydrodynamic size, especially for brain delivery. “Hydrodynamic size”, as used in this disclosure, is an effective size of a particle when it is dispersed in a fluid. In one or more embodiments, hydrodynamic size may include hydrodynamic diameter. In one or more embodiments, hydrodynamic size may include a diameter of a hypothetical hard sphere that diffuses through a fluid at a same rate as an actual particle. In one or more embodiments, hydrodynamic size may include a particle itself plus its solvation shell and any coating. In one or more embodiments, hydrodynamic size may be measured by dynamic light scattering (DLS) or nanoparticle tracking analysis (NTA). In one or more embodiments, measured value of hydrodynamic size may be depend on medium viscosity, temperature, ionic strength, and surface chemistry, all of which affect diffusion and the thickness of the bound solvent/electrical double layer. In one or more embodiment, hydrodynamic size may be used as a relevant parameter for phenomena like diffusion, circulation, filtration, and in vivo transport (e.g., through the blood-brain barrier), because hydrodynamic size governs how a particle behaves in its actual fluid environment. In some cases, nanoparticle binding element may include a linker, wherein the first end of the nanoparticle binding element may be connected to therapeutic oligomer sequence 108, therapeutic oligomer, and/or therapeutic nanoligomer via the linker. A linker may include a sequence such as AEEA (SEQ ID NO: 2) or the like thereof. In one or more embodiments, nanoparticle binding element may be configured to couple a nucleic acid-binding domain to a nanoparticle scaffold. In one or more embodiments, nanoparticle binding element may include one or more chemical handles, linkers, terminal modifications, or affinity motifs disposed on a PNA oligomer that enable covalent attachment or high-affinity association with a surface of a nanoparticle. Through this interaction, nanoparticle binding element may anchor PNA to nanoparticle scaffold, thereby forming a unified nanoparticle-conjugated construct rather than a free oligomer in solution. In one or more embodiments, nanoparticle binding element may function as a spacer or linker that controls a spatial presentation, orientation, and accessibility of nucleic acid-binding domain relative to nanoparticle surface. By positioning PNA at a defined distance from nanoparticle core, nanoparticle binding element may reduce steric hindrance and permit efficient hybridization of PNA with a complementary DNA or RNA target. In one or more embodiments, nanoparticle binding element may contribute to physicochemical stability and biological behavior of nanoligomer. In one or more embodiments, chemical composition and length of nanoparticle binding element may influence colloidal stability of nanoparticle, resistance to aggregation, and compatibility with aqueous formulations. In combination with other surface features, nanoparticle binding element may affect effective particle size, surface charge, and hydrophilicity, which in turn may influence biodistribution, cellular uptake, clearance pathways, and biocompatibility. In one or more embodiments, nanoparticle binding element may enable a modular architecture in which a common nanoparticle scaffold may be coupled to different PNA sequences through interchangeable binding elements. This modularity may permit reuse of a standardized nanoparticle chassis while varying the nucleic acid-binding domain to target different genes, transcripts, or regulatory elements.

Continuing to refer to FIG. 1, the therapeutic oligomer sequence 108, therapeutic oligomer, and/or therapeutic nanoligomer may include a delivery nanoparticle attached to the second end of the nanoparticle binding element. A “delivery nanoparticle”, as used in this disclosure, is a nanoscale vehicle engineered for a targeted transport and release of one or more therapeutic agents within a biological system. Delivery nanoparticles are typically composed of biocompatible and biodegradable materials, including lipids, polymers, and inorganic substances, and are designed to improve the pharmacokinetics and biodistribution of the one or more therapeutic agents they carry. Typical FDA GRAS (generally regarded as safe) materials used for delivery nanoparticles may include gold or zinc oxide. Delivery nanoparticles may be functionalized with targeting ligands, such as antibodies or peptides, to enhance their specificity for particular cells or tissues, thereby minimizing off-target effects and enhancing therapeutic efficacy. As a nonlimiting example, delivery nanoparticle may be configured to cross the blood-brain barrier in order to trigger a therapeutic effect. The nanoscale size of delivery nanoparticles allows them to navigate biological barriers and achieve controlled release profiles, making them a useful technology in precision medicine and targeted therapy applications. In some cases, a delivery nanoparticle may include a nanoparticle less than 2 nm in diameter. In some cases, the total hydrodynamic size of a nanoligomer formed by combining all elements of the oligomer may be kept at less than 2 nm to promote efficient transport to the brain. Nanoparticle material and size may be screened for redox potential (to screen for degradation issues), potential to generate reactive oxygen species (ROS), and some other biological redox considerations, before it is approved for further nanoligomer use. For example, in past use cases, gold nanoparticles including 22 gold atoms (Au22) and gold nanoparticles including 25 gold atoms (Au25) were selected based on the biological redox and the above screening criterion. In some cases, delivery of therapeutic oligomer/nanoligomer may not require special formulation such as lipid nanoparticle (LPN). Use of delivery nanoparticle or the like may result in a high bioavailability of therapeutic nanoligomer in a target organ such as the brain region. Use of delivery nanoparticle or the like may contribute to a rapid biodistribution of therapeutic nanoligomer with no preference for first-pass organs. In one or more embodiments, a nanoligomer may include a delivery nanoparticle that functions as a carrier for one or more PNA-based nucleic acid-binding domains. In one or more embodiments, delivery nanoparticle may include a gold nanoparticle (AuNP) core together with a surface coating or surface chemistry configured to support attachment, presentation, and in vivo delivery of PNA. In one or more embodiments, delivery nanoparticle may define biodistribution, pharmacokinetics, and clearance characteristics of nanoligomer. In one or more embodiments, size, surface composition, and physicochemical properties of nanoparticle may influence organ distribution, tissue penetration, circulation time, and elimination pathways following administration. Through these properties, delivery nanoparticle may determine absorption, distribution, metabolism, and excretion profiles that are not achievable by PNA sequence alone. In one or more embodiments, delivery nanoparticle may be engineered to support biocompatibility and low immunogenicity. Surface chemistry, particle size, and charge characteristics may be selected to minimize off-target toxicity and innate immune activation while preserving delivery efficiency. In one or more embodiments, delivery nanoparticle may enable administration of nanoligomers through multiple routes, including systemic, local, mucosal, or central nervous system delivery routes. Physical and chemical stability of nanoparticle may allow formulation for intravenous, intraperitoneal, intranasal, intracerebral, or oral delivery, depending on an intended therapeutic application.

Still referring to FIG. 1, therapeutic oligomer sequence 108, therapeutic oligomer, and/or therapeutic nanoligomer may include a coating and/or one or more surface elements to facilitate cellular uptake. Such coating and/or surface elements may function as part of a cellular uptake domain (CUD). CUD elements may include, without limitation, short amino acid coatings on nanoparticles to promote active diffusion through cell surface receptors. In a nonlimiting example, charge-neutral and/or zwitterionic amino acids may be used to prevent protein corona during transport through cell media, blood serum/others, to keep a low hydrodynamic radius for efficient transport, and prevent any immunogenic response. In some cases, no immunogenic response may be observed after administration of therapeutic nanoligomer. Amino acid binding chemistry may be tested to ensure stable binding and active diffusion. For instance, and without limitation, amino acid coatings may be tested by using less than 1-5% cell volume in vitro; testing may be expected to uptake at least 20-95% nanoligomers (benchmark) in uptake studies with specific cell types, to screen and use in further testing in vitro and/or in vivo. Stability may be tested through identification of NBD domains in free cell media, pKa, serum test, or the like. Typical examples of CUD coatings used and validated may include, without limitation, cysteine and/or glutathione with gold nanoparticles. In one or more embodiments, CUD may be defined by a combination of surface properties of a delivery nanoparticle, including nanoparticle size, core composition, surface coatings, surface charge, hydrophilicity, and any surface-displayed ligands, peptides, or chemical groups. In one or more embodiments, CUD may include the exposed portions of PNA oligomers and associated linkers presented on the surface of a gold nanoparticle, together with one or more passivating or stabilizing surface coatings. In one or more embodiments, CUD may initiate interaction with cellular membranes by enabling nonspecific or weakly specific interactions between nanoligomer and lipid bilayers. Such interactions may be driven by electrostatic forces, hydrophobic interactions, or a combination thereof, depending on surface charge density and chemical composition. In one or more embodiments, CUD may include one or more targeting ligands configured to engage cell-surface receptors, thereby enhancing association with particular cell types or tissues. In one or more embodiments, CUD may promote internalization of nanoligomer through one or more endocytic pathways. Size, surface chemistry, and ligand composition of CUD may influence the rate and extent of endocytosis, as well as the relative contribution of clathrin-mediated endocytosis, caveolin-mediated uptake, macropinocytosis, or related pathways. In one or more embodiments, CUD may be configured to support uptake across diverse cell types. In one or more embodiments, CUD may influence tissue distribution and cell-type tropism of nanoligomer. Surface properties of nanoligomer may permit uptake by cells within the central nervous system, immune system, gastrointestinal tract, respiratory epithelium, or other tissues, and may support transport across physiological barriers, including the blood-brain barrier. In one or more embodiments, CUD may facilitate productive intracellular trafficking of nanoligomer following endocytosis. Chemical composition or structural features of CUD may promote interaction with endosomal membranes, particularly under acidic conditions, thereby enabling partial escape of nanoligomer from endosomal compartments into cytosol. In one or more embodiments, CUD may further support subsequent access of nucleic acid-binding domain to cytosolic RNA targets or nuclear DNA targets. In one or more embodiments, CUD may be configured to balance cellular internalization with biocompatibility. Surface chemistry may be selected to minimize membrane disruption, complement activation, or innate immune signaling while still permitting efficient uptake. In one or more embodiments, CUD may influence systemic pharmacokinetics by modulating interactions with serum proteins and clearance pathways. In one or more embodiments, CUD may include a hydrophilic polymeric coating disposed on the nanoparticle surface to reduce aggregation and promote circulation stability, while still permitting cellular internalization. In one or more embodiments, hydrophilic polymeric coating may include poly(ethylene glycol), poly(ethylene oxide), poly(vinyl alcohol), poly(vinylpyrrolidone), poly(2-oxazoline), polysaccharides, zwitterionic polymers, poly(acrylamide), poly(amino acid) polymers, or combinations and derivatives thereof, including terminally functionalized, branched, or block-copolymer forms. In one or more embodiments, CUD may include one or more weakly charged surface groups configured to enhance membrane association without inducing cytotoxicity or inflammatory signaling. In one or more embodiments, weakly charged surface groups may include primary, secondary, or tertiary amines; protonatable heterocycles; carboxylate, phosphate, or sulfonate groups; zwitterionic moieties; pH-responsive functional groups; or combinations thereof. In one or more embodiments, CUD may include a targeting ligand selected from peptides, small molecules, carbohydrates, or antibodies configured to bind receptors expressed on neural cells, immune cells, epithelial cells, or diseased tissues. In one or more embodiments, CUD may include a pH-responsive or amphipathic surface element configured to increase interaction with endosomal membranes under acidic conditions, thereby promoting release of the nanoligomer into cytosol. In one or more embodiments, pH-responsive or amphipathic surface elements may include weakly basic moieties, protonatable heterocycles, amphipathic polymers, amphipathic peptides, or lipid-interacting segments that undergo protonation-dependent charge modulation, conformational rearrangement, or exposure of hydrophobic domains at acidic pH. In one or more embodiments, CUD may be configured to support uptake across mucosal surfaces, including nasal, gastrointestinal, or pulmonary epithelia, enabling non-parenteral routes of administration. In one or more embodiments, CUD may be optimized to support transport across the blood-brain barrier by selecting nanoparticle size, surface charge, and ligand composition that favor transcytosis or endothelial uptake.

Still referring to FIG. 1, “transport domain,” as used in this disclosure, is a functional module of a nanoligomer that governs in vivo transport, biodistribution, barrier crossing, and cellular entry of nanoligomer. In one or more embodiments, transport domain may include a transition-metal nanoparticle core together with surface-exposed elements that mediate uptake and trafficking, while excluding sequence-specific nucleic acid recognition. In one or more embodiments, a nanoligomer may include a transport domain configured to convey a nucleic acid-binding domain from a site of administration to intracellular target sites within one or more tissues. In one or more embodiments, transport domain may include a transition-metal nanoparticle core, such as a gold nanoparticle, together with surface-associated components including nanoparticle binding elements, surface coatings, and cellular uptake elements that collectively control circulation, tissue distribution, barrier traversal, and cellular internalization. In one or more embodiments, transport domain may determine systemic pharmacokinetics of nanoligomer, including circulation time, organ distribution, and clearance pathways. Physical properties of transport domain, including nanoparticle size, surface chemistry, and overall charge, may influence absorption, distribution, and elimination following administration. In one or more embodiments, transport domain may promote rapid tissue distribution with controlled clearance, thereby enabling therapeutic concentrations of nanoligomer to be achieved in target organs without prolonged systemic accumulation. In one or more embodiments, transport domain may facilitate traversal of biological barriers that limit access of free oligonucleotides, including vascular endothelium, epithelial layers, mucosal surfaces, and blood-brain barrier. Surface features of transport domain may enable translocation across such barriers following systemic, mucosal, or localized administration, thereby permitting delivery of the nucleic acid-binding domain to protected or otherwise inaccessible tissues. In one or more embodiments, transport domain may enable entry of nanoligomer into target cells by mediating membrane interaction and endocytic uptake. Surface-exposed elements of transport domain may initiate contact with cellular membranes and promote internalization through one or more endocytic pathways. In one or more embodiments, transport domain may influence intracellular trafficking of nanoligomer following uptake. Structural or chemical features of transport domain may promote release from endosomal compartments and access to cytosolic or nuclear compartments, thereby enabling the nucleic acid-binding domain to interact with its RNA or DNA targets. In one or more embodiments, transport domain may stabilize nucleic acid-binding domain through multivalent presentation on nanoparticle surface. In one or more embodiments, transition-metal nanoparticle core may support attachment of multiple PNA molecules in a defined spatial arrangement, providing a stable, multivalent carrier that enhances effective local concentration and robustness of target engagement. In one or more embodiments, transport domain may be configured to promote biocompatibility and limit immunogenicity. Surface chemistry and overall physicochemical properties of transport domain may be selected to reduce activation of innate immune responses, minimize nonspecific toxicity, and support repeated or chronic dosing.

Still referring to FIG. 1, “target sequence,” as used in this disclosure, is a functional module of a nanoligomer that contains nucleic-acid binding domain and nanoparticle binding element. In one or more embodiments, a target sequence may be a PNA-based oligomeric construct that may include a nucleic-acid binding domain operably linked to a nanoparticle binding element. In one or more embodiments, a target sequence may be synthesized as a single, continuous strand prior to conjugation to a delivery nanoparticle. In one or more embodiments, nucleic-acid binding domain may include an antisense PNA segment configured to hybridize to a predefined RNA or DNA target sequence with high affinity and sequence specificity, thereby enabling steric inhibition of transcription, translation, or nucleic-acid processing upon binding. In one or more embodiments, nanoparticle binding element may include a chemical handle or linker configured to enable stable attachment of a PNA strand to a nanoparticle surface. In one or more embodiments, target sequence may be designed to encode both biological specificity and platform compatibility, such that substitution of the nucleic-acid binding domain enables retargeting to a different gene or pathogen sequence without modification of the nanoparticle conjugation chemistry or delivery architecture.

Still referring to FIG. 1, in some embodiments, therapeutic oligomer sequence 108, therapeutic oligomer, and/or therapeutic nanoligomer may be formulated with a pharmaceutically acceptable excipient. As used herein, a “pharmaceutically acceptable excipient” is a pharmaceutically acceptable material, composition or vehicle involved in carrying or transporting a payload from one cell-type, organ, or portion of the body to another cell-type, organ, or portion of the body. Pharmaceutically acceptable excipients may include, for example and without limitation, solvents, dispersion media, coatings, antibacterial and antifungal agents, isotonic and absorption delaying agents, liquid or solid fillers, diluents, excipients, manufacturing aids (such as lubricants, talc magnesium, calcium or zinc stearate, or steric acid), or solvent-encapsulating materials. Each pharmaceutically acceptable excipient may be “acceptable” in the sense of being compatible with the other ingredients of the formulation and not injurious to the subject. Some examples of materials which may serve as pharmaceutically-acceptable excipients include, without limitation: (1) sugars, for example lactose, glucose, mannose, and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate, and/or talc; (S) excipients, for example cocoa butter and/or suppository waxes; (9) oils, for example peanut oil, cottonseed oil, safflower oil, sesame oil, olive oil, corn oil, and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or polyethylene glycol-400 (PEG400); (18) Ringer's solution; (19) C2-C12 alcohols, for example ethanol; (20) fatty acids; (21) pH buffered solutions; (22) bulking agents, for example polypeptides and/or amino acids (23) serum component, for example serum albumin, high-density lipoprotein (HDL) and/or low-density lipoprotein (LDL); (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants.

Still referring to FIG. 1, therapeutic oligomer sequence 108, therapeutic oligomer, and/or therapeutic nanoligomer may be described herein may be administered to a subject by any one of a variety of manners or a combination of varieties of manners. For example, a composition may be administered via oral administration, nasal administration, intraperitoneal administration, parenteral administration, intravenous administration, intramuscular administration, topical administration, subcutaneous administration, and/or injection into tissue, among others. In some cases, therapeutic nanoligomer may be administered with a non-toxic dosage above 500 mg/kg.

Still referring to FIG. 1, where therapeutic oligomer sequence 108 performs transcriptional downregulation, therapeutic oligomer sequence 108 may include a nuclear localization sequence (NLS). “Transcriptional downregulation”, as used herein, is a process or action whereby transcription of a genetic sequence from DNA to RNA is reduced or halted for a protein to be suppressed by therapeutic oligomer sequence. A “nuclear localization sequence”, as used in this disclosure, is a sequence of monomers that aids in insertion of an oligomer and/or nanoligomer into a nucleus of a cell. A nuclear localization sequence may include without limitation a peptide sequence. In an embodiment, this may enable the therapeutic oligomer sequence to interfere with transcription at the site where it occurs. In one or more embodiments, NLS may include a short peptide motif configured to interact with nuclear import machinery and bias intracellular transport of an associated construct toward nucleus. In one or more embodiments, NLS may include a basic amino-acid-rich sequence that is recognized by importin proteins following cytosolic entry of a nanoparticle-associated oligomer. In one or more embodiments, NLS may be operably associated with a delivery nanoparticle, a surface coating, or an oligomeric component such that NLS is surface-exposed and accessible after endosomal escape. In one or more embodiments, upon recognition by importins, NLS may facilitate docking of the associated construct at nuclear pore complexes and translocation into nuclear compartment. In one or more embodiments, incorporation of an NLS may increase nuclear accumulation of construct relative to cytosolic distribution, thereby enhancing access of a nucleic-acid binding domain to nuclear targets including pre-mRNA, nuclear-retained RNA, or genomic DNA. In one or more embodiments, NLS may modulate the kinetics and extent of nuclear entry, influencing onset, magnitude, and duration of biological activity against nuclear targets. In one or more embodiments, inclusion of an NLS may alter subcellular distribution and off-target interaction profiles, requiring sequence design and screening to maintain specificity. NLS peptide sequences may include, without limitation, sequence PKKKRKV (SEQ ID NO: 3), which is used as a nuclear localization signal by variants of simian virus 40 (SV40) and has been shown to enable localization/nuclear uptake. As a further nonlimiting example, sequences from nucleoplasmin, such as AVKRPAATKKAGQAKKKKLD (SEQ ID NO: 4), may be used for NLS. In another nonlimiting example, sequences from cellular myelocytomatosis oncogene (c-Myc) such as peptide sequence PAAKRVKLD (SEQ ID NO: 5) may be used as an NLS. As yet another nonlimiting example, an NLS may include EGL-13 SOX domain transcription factor (EGL-13) sequences such as without limitation MSRRRKANPTKLSENAKKLAKEVEN (SEQ ID NO: 6). As another nonlimiting example, NLS may include terminus utilization substance (TUS-protein) sequences such as KLKIKRPVK (SEQ ID NO: 7).

With continued reference to FIG. 1, therapeutic oligomer sequence may alternatively, or additionally, perform translational downregulation. As used in this disclosure, “translational downregulation” is a process whereby translation from a nucleotide sequence to an amino acid sequence and/or protein is inhibited and/or blocked. For instance, and without limitation, translational downregulation may be performed by preventing entry of mRNA into ribosomes, preventing mRNA from interacting with tRNA, denaturing mRNA, binding to mRNA, or the like. As a nonlimiting example, a therapeutic oligomer may bind to mRNA that codes a protein and/or amino acid sequence to be downregulated, preventing translation of that mRNA into that protein and/or amino acid sequence. In some embodiments, translational downregulation may not require entry into a cellular nucleus or other location containing DNA and/or chromosomes; transcriptional downregulation may be performed, for instance, in cytoplasm. A therapeutic oligomer performing translational downregulation may not require or include a nuclear localization sequence.

Still referring to FIG. 1, computing device 104 generates a genomic library 112 for an organism. As used in this disclosure, a “genomic library” is a database of genomic information that relates to a host organism. As used in this disclosure, “genomic information” is data and/or genetic material that codes one or more genomes. For example, and without limitation, genomic information may include DNA, RNA, and the like thereof. In an embodiment, and without limitation, genomic information may include coding regions of DNA and/or RNA and/or noncoding regions of DNA and/or RNA. In another embodiment, and without limitation, genomic information may include mitochondrial DNA and/or RNA, chloroplast DNA and/or RNA, and/or the like thereof. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively, or additionally, be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Still referring to FIG. 1, genomic library 112 may include one or more inputs such as a PNA sequence length and/or a pair of gene coordinates relative to a +1 translation start site. In an embodiment, and without limitation, genomic library 112 may include sequence warning inputs and/or STRING protein analysis inputs, wherein a sequence warning is described below in detail. In an embodiment, and without limitation, genomic library 112 for an organism may include a list of gene identifiers (IDs). For example, and without limitation, gene IDs may represent the genes of the given organism as a function of a genome assembly and/or annotation file. In another embodiment, and without limitation, genomic library 112 for an organism may include a genome assembly for a target organism, wherein a target organism may include a human organism and/or a non-human organism. In another embodiment, and without limitation, genomic library 112 for an organism may include a corresponding GFF annotation file. In an embodiment, and without limitation, genomic library 112 may include a “Get Sequences” tool that may read a gene ID and, for each list entry, search through the GFF file for any coding sequence feature (designated in GFF format as “CDS”) or parent gene feature that has a matching identifier. Upon finding a matching coding sequence, “Get Sequence” tool may extract the feature name, the start and end genomic coordinates, the feature strand, and/or any other additional information in genomic library 112.

Still referring to FIG. 1, computing device 104 generates genomic library 112 for an organism from a gene target 116. As used in this disclosure, a “gene target” is a gene of interest that is encoded in a nucleic acid sequence. In another embodiment, and without limitation, gene target 116 may include one or more nucleic acid sequences such as, but not limited to, chromosomes, plasmids, DNA, RNA, dsRNA, dsDNA, mRNA, siRNA, RNA, hpRNA, and the like thereof. As a nonlimiting example, gene target 116 may include an RNA sequencing target. In an embodiment, and without limitation gene target 116 may include a known gene target. For example, and without limitation, gene target 116 may be known as a function of a whole genome assembly. As used in this disclosure, a “whole genome assembly” is a sequence composition of an entire genome within the cell of an organism. For example, and without limitation, a whole genome assembly may be identified as a function of a whole-genome sequencing. In an embodiment, and without limitation, a whole-genome sequencing may be performed to identify a resistance factor, create a genome assembly that may be used for antisense PNA design, and/or search for genomic contributions to a resistance phenotype. For example, and without limitation, a whole-genome sequencing may be performed to determine a genome assembly for one or more viral agents such as but not limited to SARS-COV-2. For example, and without limitation, whole-genome sequencing may include using an ARG-ANNOT database, IHU Mediterranean Infection, Marseille, France, to identify a genome strain that encodes fifteen genes related to a microbiome disease. Additionally, or alternatively, whole-genome sequencing may be stored as genomic library 112.

In an embodiment, and still referring to FIG. 1, proposed therapeutic oligomer may be designed to regulate expression of a gene target 116 in a host organism to treat one or more medical conditions. For example, and without limitation, proposed therapeutic oligomer may be designed to treat a bacterial infection, such as but not limited to a multidrug-resistant (MDR) bacterial infection. As a further nonlimiting example, proposed therapeutic oligomer sequence 108 may be designed to treat and/or mitigate a microbiome disease, such as but not limited to treating one or more microbiota, and/or modulating genes associated to the microbiome disease. As a further nonlimiting example, proposed therapeutic oligomer sequence 108 may be designed to treat and/or mitigate an immune/autoimmune disease, such as but not limited to Addison's disease, celiac disease, dermatomyositis, Graves' disease, Hashimoto thyroiditis, multiple sclerosis (MS) including primary progressive multiple sclerosis (PPMS), Myasthenia gravis, Pernicious anemia, inflammatory bowel disease (IBD), Crohn's disease, ulcerative colitis, sepsis, systemic lupus erythematosus (SLE), rheumatoid arthritis, and the like thereof. As a further nonlimiting example, proposed therapeutic oligomer sequence 108 may be designed to treat and/or mitigate an oncological disease, such as but not limited to breast cancer, lung cancer including small cell lung cancer, colon cancer, rectum cancer, prostate cancer, skin cancer, stomach cancer, and the like thereof. As a further nonlimiting example, proposed therapeutic oligomer sequence 108 may be designed to treat and/or mitigate a protein disorder and/or protein-misfolding disease (NPMD), such as but not limited to Parkinson's disease, Huntington's disease, Creutzfeldt-Jakob disease, cystic fibrosis, Gaucher's disease, and the like thereof. As a further nonlimiting example, proposed therapeutic oligomer sequence 108 may be designed to treat and/or mitigate a neurodegenerative disease such as but not limited to an ethanol-induced neurodegenerative disease, microgravity-induced neuropathology, frontotemporal dementia (FTD), Alzheimer's disease (AD) including AD-related diseases (ADRDs), MS, amyotrophic lateral sclerosis (ALS), Parkinson's disease, Prion disease, and/or the like thereof. As a further nonlimiting example, proposed therapeutic oligomer sequence 108 may be designed to treat and/or mitigate an infectious disease, such as but not limited to chickenpox, influenza, diphtheria, giardiasis, infectious mononucleosis, Herpes Simplex Virus 1, Herpes Simplex Virus 2, syphilis, shigellosis, chlamydia, influenza A, influenza B, SARS-COV-2, COVID-19, and the like thereof. As a further nonlimiting example, proposed therapeutic oligomer sequence 108 may be designed to treat and/or mitigate a cardiometabolic disease, such as but not limited to diabetes, liver fibrosis, liver damage, nonalcoholic steatohepatitis (NASH), among others. As a further nonlimiting example, proposed therapeutic oligomer sequence 108 may be designed to treat joint pain or gout, among others. As a further nonlimiting example, proposed therapeutic oligomer sequence 108 may be designed to treat and/or mitigate an age-related disease such as but not limited to myocardial infarction (MI) or heart attack. Additionally, or alternatively, proposed therapeutic oligomer sequence 108 may be designed to treat and/or mitigate a viral agent and/or infectious agent such as a bacterium, virus, and the like thereof.

In an embodiment, and still referring to FIG. 1, gene target 116 may be known as a function of a partial genome assembly. As used in this disclosure, a “partial genome assembly” is a sequence composition of a portion of a genome within the cell of an organism. For example, and without limitation, a partial genome assembly may be identified as a function of a partial-genome sequencing. For example, and without limitation, partial-genome sequencing may include performing small RNA sequencing to search for potential PNA targets among short nucleic acids potentially involved in gene regulation. In an embodiment, and without limitation, a small RNA may influence pathogen response and/or viral agent response. In another embodiment, and without limitation, a small RNA may be isolated as a function of an RNA isolation protocol enriched for sRNA prior to sequencing, wherein sequencing data may allow for identification of previously documented sRNA and/or novel RNAs. In an embodiment, and without limitation, a small RNA sequencing may determine an overlap of differentially expressed (DE) genes between single time points. Additionally or alternatively, small RNA sequencing may identify 22 sRNAs, such as but not limited to known regulatory sRNAs (dicF, ssrA), annotated short protein coding genes (ilvB, acpP, bolA, csrA, ihfA, lspA), small putative protein-coding genes (dsrB, yahM, ybcJ, ygdI, ygdR, ytfK), small transcripts antisense to coding genes (ygaC, hemN, ECUMN_1534/5), and/or novel predicted transcripts. In an embodiment, and without limitation, small RNA sequencing may allow gene target selection such as, but not limited to selecting three genes of interest, wherein the three genes of interest may include without limitation bolA, dsrB, ygaC, and/or the like thereof. Additionally, or alternatively, The RNA sequencing and/or partial genome sequencing may be stored in genomic library 112. Additionally, or alternatively, a partial genome assembly may denote one or more genomic sequence compositions that code for a microbiome disease, immune disease, oncological disease, protein disorder, neurodegenerative disease, infectious disease, and the like thereof.

Still referring to FIG. 1, a gene target 116 may include proinflammatory cytokines. Gene target 116 and/or proinflammatory cytokines may include Interleukin-1 (IL-1) such as Interleukin-1α (IL-1α) and Interleukin-1β (IL-1β), tumor necrosis factor alpha (TNF-α), tumor necrosis factor-alpha receptor 1 (TNF-R1), Interleukin-4 (IL-4), Interleukin-6 (IL-6), Interleukin-10 (IL-10), Interleukin-13 (IL-13), Interleukin-18 (IL-18), Interleukin-31 (IL-31). It is worth noting that IL-1 and IL-18 have been linked to autoinflammatory and autoimmune disorders. Accordingly, by targeting one or more these gene targets, therapeutic nanoligomer may be used to treat a host with neurodegenerative and/or autoimmune disorders. Gene target 116 and/or proinflammatory cytokines may include inflammasomes including NOD-like receptor family, pyrin domain containing 1 (NLRP1), NOD-like receptor family, pyrin domain containing 3 (NLRP3), NOD-like receptor family, pyrin domain containing 4 (NLRP4), NOD-like receptor family, pyrin domain containing 6 (NLRP6), absent in melanoma 2 (Interferon-inducible protein AIM2 or simply AIM2), granulocyte-macrophage colony-stimulating factor (GM-CSF or CSF-2), granulocyte colony-stimulating factor (G-CSF), key transcription factors such as nuclear factor kappa-light-chain-enhancer of activated B cells (nuclear factor kappa-B or NF-κB), and their combinations, as upstream regulators and canonical pathway targets, to identify and validate the best-in-class treatment. Accordingly, in some cases, therapeutic nanoligomer may perform its function by acting as an inflammasome inhibitor. Cytokines may be regulators of host responses to infection, immune responses, inflammation, and trauma. Some cytokines may act to make disease worse (proinflammatory), whereas others may serve to reduce inflammation and promote healing (anti-inflammatory). Proinflammatory cytokines may affect a plurality of the host in a plurality of ways including creating neuroinflammation. Additionally, a gene target 116 may include an inflammasome acquisition frenzy cytokine. Examples of inflammasome acquisition frenzy cytokines may include without limitation NLRP3 and NF-κB inhibitors. In some cases, gene target 116 may include erythropoietin (EPO), gamma globulin (GG), TAR DNA binding protein 43 (TDP-43), telomerase reverse transcriptase (TERT), chemokine ligands 4 (CCL4), chemokine ligands 20 (CCL20), C—X—C motif chemokine 5 (CXCL5), C—X—C motif chemokine 11 (CXCL11), CD40 ligand (CD40L), and/or TNF receptor superfamily member 8 (TNFRSF8 or CD30). In some cases, gene target 116 may include a microRNA, such as miR-2392 for COVID-19. In some cases, therapeutic oligomer sequence 108, therapeutic oligomer, and/or therapeutic nanoligomer may target a plurality of gene targets 116 to achieve a synergistic effect. As a nonlimiting example, therapeutic oligomer sequence 108, therapeutic oligomer, and/or therapeutic nanoligomer may target both NLRP3 and NF-κB to treat a neurodegenerative disorder and/or improve cognitive function with aging and tauopathy. Additional details will be provided below in this disclosure. As another nonlimiting example, therapeutic oligomer sequence 108, therapeutic oligomer, and/or therapeutic nanoligomer may target both TNF-R1 and NF-κβ to treat a case of neuroinflammation. Additional details will be provided below in this disclosure. As another nonlimiting example, therapeutic oligomer sequence 108, therapeutic oligomer, and/or therapeutic nanoligomer may target both IL-6 and NF-κβ to treat a case of microgravity-induced neuropathology. Additional details will be provided below in this disclosure.

Still referring to FIG. 1, in some embodiments, a nanoligomer may be capable of regulating NLRP3 inflammasome. “NLRP3 inflammasome,” as used in this disclosure, is an intracellular, cytosolic multiprotein signaling complex. In one or more embodiments, NLRP3 inflammasome may function as an innate immune sensor linking cellular stress signals to inflammatory effector pathways. In one or more embodiments, NLRP3 inflammasome may include a sensor protein NLRP3, an adaptor protein ASC, and an effector protease precursor pro-caspase-1, which together assemble into an active signaling platform in response to defined priming and activation cues. In one or more embodiments, activation of NLRP3 inflammasome may result in conversion of pro-caspase-1 to active caspase-1, followed by proteolytic processing of pro-inflammatory cytokines into mature IL-1β and IL-18 and induction of inflammatory cell death through pyroptosis. In one or more embodiments, NLRP3 inflammasome activation may be initiated through a priming process that increases expression of inflammasome components and cytokine precursors, followed by an activation process triggered by intracellular stress signals such as ionic flux, mitochondrial dysfunction, reactive oxygen species, or lysosomal disruption. In one or more embodiments, NLRP3 inflammasome functions as a central regulatory hub that integrates diverse danger-associated or pathogen-associated signals to drive downstream inflammatory responses. In one or more embodiments, dysregulated or sustained activation of NLRP3 inflammasome may contribute to pathological inflammation and tissue damage. In some embodiments, a nanoligomer may be capable of downregulating expression of NLRP3 gene. In some embodiments, a nanoligomer may include a targeting sequence capable of hybridizing with a polynucleotide encoding NLRP3. In some embodiments, a nanoligomer may include a targeting sequence capable of hybridizing with mRNA encoding NLRP3. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NLRP3. In some embodiments, a nanoligomer may be capable of downregulating expression of the NF-κβ gene. In some embodiments, a nanoligomer may include a targeting sequence capable of hybridizing with a polynucleotide encoding NF-κB. In some embodiments, a nanoligomer may include a targeting sequence capable of hybridizing with mRNA encoding NF-κB. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NF-κB. In some embodiments, a nanoligomer may include a targeting sequence capable of hybridizing with a polynucleotide encoding a gene selected from IL-1β, IL-1α, TNF-α, IL-6, IL-4, IL-13, AIM2, TNRF1, NLRP1, NLRP6, NLRC4, NLRP3, and NF-κB.

Still referring to FIG. 1, in some embodiments, a composition including a nanoligomer targeting NLRP3 and a nanoligomer targeting NF-κβ is administered to a subject. In some embodiments, administration of a NLRP3 nanoligomer and a NF-κβ nanoligomer may lead to a reduction in IL-18 levels compared to a nanoligomer targeting only one of them or compared to a control. In some embodiments, administration of a NLRP3 nanoligomer and a NF-κB nanoligomer may lead to a reduction in IL-1 levels compared to a nanoligomer targeting only one of them or compared to a control. In some embodiments, administration of a NLRP3 nanoligomer and a NF-κβ nanoligomer may lead to a reduction in IL-4 levels compared to a nanoligomer targeting only one of them or compared to a control. In some embodiments, administration of a NLRP3 nanoligomer and a NF-κβ nanoligomer may lead to a reduction in CD30 levels compared to a nanoligomer targeting only one of them or compared to a control. In some embodiments, administration of a NLRP3 nanoligomer and a NF-κβ nanoligomer may lead to a reduction in IL-31 levels compared to a nanoligomer targeting only one of them or compared to a control. In some embodiments, administration of a NLRP3 nanoligomer and a NF-κβ nanoligomer may lead to a reduction in CXCL5 levels compared to a nanoligomer targeting only one of them or compared to a control. In some embodiments, administration of a NLRP3 nanoligomer and a NF-κβ nanoligomer may lead to a reduction in CCL4 levels compared to a nanoligomer targeting only one of them or compared to a control. In some embodiments, administration of a NLRP3 nanoligomer and a NF-κβ nanoligomer may lead to a reduction in CCL20 levels compared to a nanoligomer targeting only one of them or compared to a control. In some embodiments, administration of a NLRP3 nanoligomer and a NF-κβ nanoligomer may lead to a reduction in CXCL11 levels compared to a nanoligomer targeting only one of them or compared to a control. In some embodiments, administration of a NLRP3 nanoligomer and a NF-κβ nanoligomer may lead to a reduction in CD40L levels compared to a nanoligomer targeting only one of them or compared to a control.

Still referring to FIG. 1, a composition described herein may be administered to a subject that has a disease, or that has a risk of developing a disease. As an example, a composition described herein may be administered to a subject that has a disease associated with neuroinflammation. A composition described herein may be administered to a subject that has a disease selected from the list IBD, Alzheimer's disease, Parkinson's disease, multiple sclerosis, prion's disease/Creutzfeldt-Jakob disease (CJD), neurodegenerative diseases, autoimmune diseases, cancer, liver fibrosis, NASH, diabetes, gout, myocardial infarction, and sepsis. In some embodiments, a disease may be treated via a method including administering to a subject one or more nanoligomers, or a composition including one or more nanoligomers. For example, a disease may be treated by administering a composition comprising a nanoligomer targeting NF-κB DNA or mRNA, and a nanoligomer targeting NLRP3 DNA or mRNA. As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Still referring to FIG. 1, in some embodiments, a composition described herein may be administered to a subject that has inflammatory bowel disease (IBD). In some embodiments, a composition described herein may be administered to a subject that has a risk of developing IBD. In some embodiments, IBD may refer to Crohn's disease and ulcerative colitis. In some embodiments, IBD may be characterized by chronic inflammation of the GI tract. In some embodiments, a composition described herein may be administered to a subject that has Crohn's disease. In some embodiments, a composition described herein may be administered to a subject that has ulcerative colitis.

Still referring to FIG. 1, in some embodiments, a nanoligomer may include a nanoligomer disclosed in Table 1.

TABLE 1

| | | SEQ ID |
|---|---|---|
| Description | Sequence | NO. |
| SB_NI_112 | AGTGGTACCGTCTGCTA-AEEA- | 25, 2 |
| mouse NFKB1 | HHHHH-Au22, Glutathione 18 | and 1 |
| SB_NI_112 | CTTCTACTGCTCACAGG-AEEA- | 26, 2 |
| mouse NLRP3 | HHHHH-Au22, Glutathione 18 | and 1 |
| SB_NI_112 | CGGGTGCTTGCCATCTT-AEEA- | 27, 2 |
| human NLRP3 | HHHHH-Au22, Glutathione 18 | and 1 |
| SB_NI_112 | TGCCATTCTGAAGCCGG-AEEA- | 28, 2 |
| human NF-κβ | HHHHH-Au22, Glutathione 18 | and 1 |

Still referring to FIG. 1, in some embodiments, a nanoligomer may include a nanoligomer disclosed in Table 2.

TABLE 2

| Description | Sequence | SEQ ID NO. |
|---|---|---|
| SB_BGC_CK1- | ATCACCAAGTAG-PO- | 50 and 10 |
| A | KFFKFFKFFK | |
| SB_BGC_CK1- | TGTGTTACGCTA-PO- | 51 and 10 |
| B | KFFKFFKFFK | |
| SB_BGC_CK1- | GTGACATACATT-PO- | 52 and 10 |
| C | KFFKFFKFFK | |

As used in Table 2, A, T, G, and C are nucleotides on a peptide nucleic acid backbone. As used in Table 2, K is Lysine and F is Phenylalanine. As used in Table 2, PO is an AEEA (SEQ ID NO: 2) linker. In some embodiments, a composition may include one or more nanoligomers described in Table 2. For example, a composition may include all 3 nanoligomers described in Table 2.

Still referring to FIG. 1, in some embodiments, a nanoligomer may include a targeting sequence. As used herein, a “targeting sequence” is a sequence of nucleobases including a polynucleotide binding domain and a nanostructure binding domain. A targeting sequence may include a peptide nucleic acid (PNA). As used in this disclosure, a “peptide nucleic acid” is a DNA analog comprising a (2-aminoethyl)glycine carbonyl unit, as opposed to a phosphate backbone, that is linked to a nucleotide base by the glycine amino nitrogen and/or methylene linker. In an embodiment, and without limitation, a PNA may include a backbone composed of peptide bonds linking nucleobases. In another embodiment, and without limitation, a PNA may include an amino-terminal and/or a carboxy-terminal end. In another embodiment, and without limitation, a PNA may include a 5′ and/or a 3′ end in the conventional sense, with reference to a complementary nucleic acid sequence to which it specifically hybridizes. In an embodiment, a PNA may include a sequence that may be described in a conventional fashion similar to DNA and/or RNA, such as but not limited to having nucleotides including guanine (G), uracil (U), thymine (T), adenine (A), and/or cytosine (C) which may correspond to a nucleotide sequence of a DNA molecule. In an embodiment, a PNA may be resistant to proteases and/or nucleases as a function of a structural difference from DNA, wherein the structural difference may result in a PNA not being recognized by a hepatic transporter(s) recognizing DNA. In another embodiment, a PNA may comprise at least one modified phosphate backbone such as, but not limited to phosphorothioate, phosphorodithioate, 5-phosphoramidothioate, phosphoramidate, phosphordiamidate, methylphosphonate, alkyl phosphotriester, formacetal, and/or the like thereof. In some embodiments, one or more of a polynucleotide binding domain, a nanoparticle binding domain, a nuclear localization sequence, and a transcription activation domain includes a PNA. A targeting sequence may include a polynucleotide, such as DNA or RNA. A targeting sequence may include an antisense oligonucleotide. As used in this disclosure, an “antisense oligonucleotide” is an antisense molecule that modulates the expression of one or more genes and/or polynucleotides. For example, and without limitation, an antisense oligonucleotide may include antisense PNAs, antisense RNAs, and the like. In another embodiment, antisense oligonucleotides may include RNA and/or DNA oligomers such as but not limited to interfering RNA molecules, such as dsRNA, dsDNA, RNA, siRNA, and/or hpRNA as well as locked nucleic acids, BNA, polypeptides and/or other oligomers and the like. A nanoligomer may include an inhibitory sequence. An “inhibitory sequence,” as used in this disclosure, is a sequence of nucleotides or other repeating units that acts to suppress a sequence of interest such as a sequence involved in the production of an undesirable protein. In a non-limiting example, an inhibitory sequence may be used to decrease production of neuroinflammatory such as proteins. An inhibitory sequence may include, without limitation, sequences of ribonucleic acid (RNA), deoxyribonucleic acid (DNA), or peptide nucleic acid (PNA). In an embodiment, a targeting sequence is complementary to a sequence of interest. In an embodiment, an antisense oligonucleotide is complementary to a sequence of interest. In an embodiment, an inhibitory sequence is complementary to a sequence of interest. As used in this disclosure, a “complementary” sequence is a sequence of consecutive nucleobases or semi-consecutive nucleobases capable of hybridizing to another nucleic acid strand or duplex even if less than all the nucleobases base pair with a counterpart nucleobase. In some embodiments, between 70% and 100%, or any range derivable therein, of a nucleobase sequence may be capable of base-pairing with a nucleic acid molecule during hybridization.

Still referring to FIG. 1, a targeting sequence may include a polynucleotide binding domain. As used herein, a “polynucleotide binding domain” is a sequence of nucleobases capable of hybridizing with a target polynucleotide. In some embodiments, a polynucleotide binding domain is complementary to a target polynucleotide. In some embodiments, a target polynucleotide encodes a proinflammatory cytokine, a direct inflammasome target, or a transcription factor. In some embodiments, a target polynucleotide encodes TERT, a cytokine selected from the list Interleukin-1β or IL-1β, IL-1α, tumor necrosis factor-alpha or TNF-α, TNF receptor 1 or TNFR1, Interleukin 6 or IL-6, IL-4, and IL-13. In some embodiments, a target polynucleotide encodes an inflammasome target selected from the list NLRP1, NLRP3, NLRC4, AIM2. In some embodiments, a target polynucleotide may encode NLRP3. In some embodiments, a target polynucleotide may encode NF-κB. In some embodiments, a polynucleotide binding domain targeting mouse NFκB1 has the sequence AGTGGTACCGTCTGCTA (SEQ ID NO: 39). In some embodiments, a polynucleotide binding domain targeting mouse NLRP3 has the sequence CTTCTACTGCTCACAGG (SEQ ID NO: 40). In some embodiments, a polynucleotide binding domain targeting human NLRP3 has the sequence CGGGTGCTTGCCATCTT (SEQ ID NO: 20). In some embodiments, a polynucleotide binding domain targeting human NF-κβ has the sequence TGCCATTCTGAAGCCGG (SEQ ID NO: 35). In some embodiments, a target polynucleotide may include a sequence disclosed in Table 3. In some embodiments, a polynucleotide binding domain may be capable of hybridizing with a sequence disclosed in Table 3.

### Table 3:

| | | SEQ |
|---|---|---|
| | | ID |
| Description | Sequence | NO. |
| Human NLRP3 RNA | CGGGTGCTTGCCATCTT | 29 |
| Human NLRP3 RNA | GTGCTTGCCATCTTCAT | 30 |
| Human NLRP3 RNA | CTTGCCATCTTCATCTG | 31 |
| Human NLRP3 RNA | CCATCTTCATCTGCAGC | 32 |
| Human NLRP3 RNA | CAGCGGGTGCTTGCCAT | 33 |
| Human NF-κβ RNA | TTCTGCCATTCTGAAGC | 34 |
| Human NF-κβ RNA | TGCCATTCTGAAGCCGG | 35 |
| Human NF-κβ RNA | CCATTCTGAAGCCGGGT | 36 |
| Human NF-κβ RNA | ATCATCTTCTGCCATTC | 37 |
| Human NF-κβ RNA | ATCTTCTGCCATTCTGA | 38 |
| Mouse NFKB1 RNA | AGTGGTACCGTCTGCTA | 39 |
| Mouse NLRP3 RNA | CTTCTACTGCTCACAGG | 40 |
| SB BGC CK1-A | ATCACCAAGTAG | 46 |
| SB BGC CK1-B | TGTGTTACGCTA | 47 |
| SB BGC CK1-C | GTGACATACATT | 48 |

Still referring to FIG. 1, in some embodiments, a polynucleotide binding sequence may be capable of hybridizing with a section of a sequence in Table 3. In non-limiting examples, a polynucleotide binding sequence may be capable of hybridizing to a 10, 11, 12, 13, 14, 15, 16, or 17 nucleotide long stretch of a sequence in Table 3.

Still referring to FIG. 1, in some embodiments, a polynucleotide binding sequence may include a sequence selected from SEQ ID NO: 25-28. In some embodiments, a polynucleotide binding sequence may be capable of hybridizing with a sequence selected from SEQ ID NO: 29-40.

Still referring to FIG. 1, in some embodiments, a polynucleotide binding sequence may be capable of hybridizing to a viral target sequence. In some embodiments, a viral target sequence may include a target sequence from a DNA virus, such as adenoviruses, herpesviruses, poxviruses, parvoviruses and the like. In some embodiments, a viral target sequence may include a target sequence from an RNA virus, such as influenza, SARS, MERS, COVID-19, Dengue Virus, hepatitis C, hepatitis E, West Nile fever, Ebola virus disease, rabies, polio, mumps, measles, and the like. In some embodiments, a polynucleotide binding sequence capable of hybridizing to a viral target sequence may include a sequence disclosed in Table 4.

TABLE 4

| Description | Sequence | SEQ ID NO. |
|---|---|---|
| α-TRS | TAAAGTTCGTTTAGA | 41 |
| α-AUG | GCTCTCCATCTTACC | 42 |
| α-FS | ACACCGCAAACCCGT | 43 |
| α-PK1 | CGGGCTGCACTTACA | 44 |
| α-PK2 | TACTAGTGCCTGTGC | 45 |
| α-PK3 | GTATACGACATCAGT | 46 |

Still referring to FIG. 1, in some embodiments, a polynucleotide binding domain may target a bacterial polynucleotide. In some embodiments, a polynucleotide binding domain may target a biosynthetic gene cluster polynucleotide. In some embodiments, a polynucleotide binding domain may target a porogymonas gingivali, eubacterium rectale, pseudobutyrivibrio xylanivorans and/or akkermansia muciniphilia polynucleotide.

Still referring to FIG. 1, in some embodiments, a target polynucleotide may include RNA, such as mRNA. In some embodiments, hybridization of a polynucleotide binding sequence to a target mRNA may sterically hinder a ribosomal binding site and downregulate translation from the mRNA. In some embodiments, hybridization of a polynucleotide binding sequence to a target mRNA may prevent translation of the RNA. In some embodiments, a target polynucleotide may include DNA. In some embodiments, hybridization of a polynucleotide binding sequence to a target DNA may prevent transcription of the DNA.

Still referring to FIG. 1, a targeting sequence 104 may include a nanostructure binding domain 116. As used herein, a “nanostructure binding domain” is a nucleobase sequence containing a binding domain for a nanostructure. In some embodiments, a nanostructure binding domain may include a sequence of 5, 6, or 7 consecutive histidine (H in Table 1), 5, 6, or 7 consecutive cysteine, 5, 6, or 7 methionine, 5, 6, or 7 lysine, 5, 6, or 7 glutamine, 5, 6, or 7 arginine, or 5, 6, or 7 asparagine. In some embodiments, a nanostructure binding domain may include a sequence of 5 consecutive histidine. In some embodiments, a nanostructure binding domain may include SEQ ID NO: 1.

Still referring to FIG. 1, a targeting sequence 104 may include a linker. As used herein, a protein “linker” is an amino acid sequence that connects two other sequences. In some embodiments, a linker may include the sequence AEEA. In some embodiments, a linker may include SEQ ID NO: 2. In some embodiments, a spacer may be positioned between nanostructure binding domain and the rest of the targeting sequence.

Still referring to FIG. 1, a nanoligomer may include a nuclear localization sequence. In some embodiments, targeting sequence 104 may include a nuclear localization sequence. As used herein, a “nuclear localization sequence” is an amino acid sequence that increases transport to the nucleus. In some embodiments, a targeting sequence includes a polynucleotide binding sequence targeting DNA and a nuclear localization sequence. In some embodiments, a targeting sequence includes a polynucleotide binding sequence targeting RNA and no nuclear localization sequence. Non-limiting examples of nuclear localization sequences include SV40 sequences such as PKKKRKV (SEQ ID NO: 3), nucleoplasmin sequences such as AVKRPAATKKAGQAKKKKLD (SEQ ID NO: 4), c-Myc sequences such as PAAKRVKLD (SEQ ID NO: 5), EGL-13 sequences such as MSRRRKANPTKLSENAKKLAKEVEN (SEQ ID NO: 6), and TUS-protein sequences such as KLKIKRPVK (SEQ ID NO: 7).

Acidic domains may be referred to as acid blobs, negative noodles, or nine-amino-acid transactivation domains (9aaTAD). Acidic domains may include a domain common to large superfamilies in eukaryotic transcription factors represented by Gal4, Oaf1, Leu3, Rtg3, Pho4, Gln3, Gcn4 in yeast, and p53, NFAT, NF-κB, and VP16 in mammals. Acidic domains may be rich in D and E amino acids. Acidic domains may have an associated 3 amino acid hydrophobic region next to its N-terminal end. In some embodiments, an acidic domain may include a 9aaTAD disclosed in Table 5.

TABLE 5

| 9 amino acid | SEQ | | |
|---|---|---|---|
| sequence | ID | | Peptide-KIX |
| (9aaTAD) | NO. | Source | interaction |
| E TFSD LWKL | 12 | p53 TAD1 | LSPEETFSDLWKLPE |
| D DIEQ WFTE | 13 | p53 TAD2 | QAMDDLMLSPDDIEQ |
| | | | WFTEDPGPD |
| S DIMD FVLK | 14 | MLL | DCGNILPSDIMDFVL |
| | | | KNTP |
| D LLDF SMMF | 15 | E2A | PVGTDKELSDLLDFS |
| | | | MMFPLPVT |
| E TLDF SLVT | 16 | Rtg3 | E2A homolog |
| R KILN DLSS | 17 | CREB | RREILSRRPSYRKIL |
| | | | NDLSSDAP |
| E AILA ELKK | 18 | CREBaB6 | CREB-mutant |
| | | | binding to KIX |
| D DVVQ YLNS | 19 | Gli3 | TAD homology to |
| | | | CREB/KIX |
| D DVYN YLFD | 20 | Gal4 | Pdr1 and Oaf1 |
| | | | homolog |
| D LFDY DFLV | 21 | Oaf1 | DLFDYDFLV |
| D FFDY DLLF | 22 | Pip2 | Oaf1 homolog |
| EDLYS ILWS | 23 | Pdr1 | EDLYSILWSDWY |
| T DLYH TLWN | 24 | Pdr3 | Pdr1 homolog |

Still referring to FIG. 1, in some embodiments, a transcription activation domain may include a glutamine rich domain. As used herein, a “glutamine rich domain” is a sequence at least 3 amino acids in length and including at least 60% glutamine. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% glutamine. In some embodiments, a transcription activation domain may include 0.01%-99.9% glutamine. In some embodiments, a transcription activation domain may include a sequence of 3-9 amino acids and at least 60% glutamine. A glutamine rich domain may contain multiple repetitions such as QQQXXXQQQ. As non-limiting examples, glutamine rich domains may include POU2F1 (Oct1), POU2F2 (Oct2), and SP1 (Sp/KLF family). In some embodiments, a glutamine rich domain may include the sequence AQQAQQQQQNQAQQAQQQQQNQ (SEQ ID NO: 47).

Still referring to FIG. 1, in some embodiments, a transcription activation domain may include a proline rich domain. As used herein, a “proline rich domain” is a sequence at least 3 amino acids in length and including at least 60% proline. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% proline. In some embodiments, a transcription activation domain may include 0.01%-99.9% proline. In some embodiments, a transcription activation domain may include a sequence of 3-9 amino acids and at least 60% proline. A proline rich domain may contain multiple repetitions such as PPPXXXPPP. As non-limiting examples, proline rich domains may include c-jun, AP2, and/or Oct-2. In some embodiments, a proline rich domain may include the sequence PPPDLGPPPDLGPPP (SEQ ID NO: 48).

Still referring to FIG. 1, in some embodiments, a transcription activation domain may include an isoleucine rich domain. As used herein, a “isoleucine rich domain” is a sequence at least 3 amino acids in length and including at least 50% isoleucine. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% isoleucine. In some embodiments, a transcription activation domain may include 0.01%-99.9% isoleucine. In some embodiments, a transcription activation domain may include a sequence of 3-9 amino acids and at least 50% isoleucine. An isoleucine rich domain may contain multiple repetitions such as IIXXII. As a non-limiting example, an isoleucine rich domain may include NTF-1. In some embodiments, an isoleucine rich domain may include the sequence KSHAHAQKRIRRRLLIILL (SEQ ID NO: 49).

Reverse transcriptase quantitative PCR analysis was used to determine mRNA levels. RNA was extracted from cell culture 6-well dishes using cell scraping, QIAshredder and RNeasy extraction kits, in accordance with manufacturer's protocol, including a DNase digestion step with the RNase-free DNase kit (Qiagen, Valencia, CA). Purity and concentration were determined using a ND-1000 spectrophotometer (NanoDrop Technologies, Wilmington, DE). Following isolation and purification, 25 ng of RNA was reverse transcribed using the iScript Reverse Transcriptase kit (BioRad, Hercules CA). The cDNA was amplified within 24 hours of reverse transcription using iQ SYBR Green Supermix (BioRad, Hercules CA). The corresponding validated primer sequences were used for each gene at 10 μM. The expression data was analyzed using the 2-ΔΔCT method and normalized to expression of reference genes β-actin. The fold difference was compared to control (normal brain homogenate treated) samples (27). Validated primer sequences are as follows:

(β-actin)
(SEQ ID NO: 53)
5′-CCACTGTCGAGTCGCGT-3′,
(forward)
(reverse)
(SEQ ID NO: 54)
5′-CGCAGCGATATCGTCATCCAT-3′;
(NLRP3)
(SEQ ID NO: 55)
5′-CCTGGGGGACTTTGGAATCA-3′,
(forward)
(SEQ ID NO: 56)
5′-GACAACACGCGGATGTGAGA-3′
(reverse);
(IL1β)
(SEQ ID NO: 57)
5′-GCAGCAGCACATCAACAAG-3′
(forward),
(reverse)
(SEQ ID NO: 58)
5′-CACGGGAAAGACACAGGTAG-3′;
(NF-κB1)
(SEQ ID NO: 59)
5′-GTGGAGGCATGTTCGGTAGT-3′
(forward),
(reverse)
(SEQ ID NO: 60)
5′-CCTGCGTTGGATTTCGTGAC-3′;
(TNFR1a)
(SEQ ID NO: 61)
5′-GTTGTCAATTGCTGCCCTGTC-3′,
(forward)
(reverse)
(SEQ ID NO: 62)
5′-CAGTGACCCCTGATGGATGT-3′.
All RT-PCR was done following MIQE guidelines.

The system disclosed herein may include one or more characteristics as described in U.S. Nonprovisional application Ser. No. 18/137,101, filed on Apr. 20, 2023, and entitled “METHODS AND SYSTEMS FOR TARGETING AUTOIMMUNE AND INFLAMMATORY PATHWAYS USING NANOLIGOMERS,” and having attorney docket no. 1186-006USU1; U.S. Nonprovisional application Ser. No. 18/376,197, filed on Oct. 3, 2023, and entitled “METHODS AND SYSTEMS FOR TARGETING AUTOIMMUNE AND INFLAMMATORY PATHWAYS USING NANOLIGOMERS,” and having attorney docket no. 1186-006USC1, all of these applications are incorporated by reference in this disclosure in their entirety.

Still referring to FIG. 1, gene target 116 may be determined as a function of a biosynthetic gene cluster derived from a microbiome gene analysis. As used in this disclosure, a “biosynthetic gene cluster” is a linked set of genes that participate in a common biosynthetic pathway. As used in this disclosure, a “microbiome gene analysis” is a gene analysis of microbiota. In an embodiment, and without limitation, a microbiome gene analysis may analyze one or more microbiota present in an individual's gastrointestinal tract. In another embodiment, and without limitation, a microbiome gene analysis may determine one or more promoters in bacteria. For example, and without limitation, a promoter may include T7, Sp6, lac, araBad, trp, Ptac, and the like thereof. For example, and without limitation, microbiome gene analysis may include packing five colonies of microbiota from a plate and resuspended in liquid growth media. A “plate”, as used herein, is a shallow, cylindrical, lidded dish that is used to culture cells. For example, and without limitation, a plate may include a petri-dish such as, but not limited to a glass petri-dish, plastic petri-dish, and/or the like thereof. Plate may include any suitable dish to produce an agar plate. As used in this disclosure, an “agar plate” is a petri-dish that includes a growth medium solidified with agar. A “growth medium”, as used herein, is a solid, liquid, and/or semi-solid material designed to support the growth of a population of microorganisms and/or cells. In an embodiment, and without limitation, a growth medium may aid microorganisms and/or cells in cell proliferation. In another embodiment, and without limitation, a agar plate may include one or more agar plates such as, but not limited to a blood agar plate, chocolate agar plate, horse blood agar plate, Thayer-Martin agar plate, thiosulfate-citrate-bile salts-sucrose agar plate, bile esculin agar, cysteine lactose electrolyte-deficient agar plate, Granada medium agar plate, Hektoen enteric agar plate, lysogeny broth agar plate, MacConkey agar plate, mannitol salt agar plate, Mueller-Hinton agar plate, nutrient agar plate, Önöz agar plate, phenethyl alcohol agar plate, R2A agar plate, tryptic soy agar plate, xylose-lysine-deoxycholate agar plate, cetrimide agar plate, tinsdale agar plate, sabouraud agar plate, hay infusion agar plate, potato dextrose agar plate, Knop agar plate, YEPD media agar plate, and/or the like thereof.

In an embodiment, and still referring to FIG. 1, microbiome gene analysis may include isolating genomic DNA as a function of a purification kit, such as but not limited to a Wizard DNA Purification Kit (Promega, Madison, Wisconsin, U.S.A). In another embodiment, microbiome gene analysis may include extracting RNA as a function of thawing a sample and resuspending the sample in 100 μL TE buffer with 0.4 mg/mL lysozyme and proteinase K, wherein after incubation at room temperature for 5 minutes, 300 μL of lysis buffer with 20 μL/mL β-mercaptoethanol may be added to each and vortexed to mix. Each lysis solution may be split in half, with one half being processed for total RNA isolation followed by DNase treatment with the TURBO DNA-free kit (Ambion, Austin TX, U.S.A.). Small RNA may be isolated using the mir Vana miRNA isolation kit (Thermo Scientific, Waltham, MA, U.S.A.). Concentration and absorbance (A260/A280) may be measured on a Nanodrop 2000 (Thermo Scientific, Waltham, MA, U.S.A.). In another embodiment, microbiome gene analysis may include preparing a sequencing library with library kit, wherein the sequencing library is sequenced as a function of an analyzer such as, but not limited to an Illumina MiSeq (Illumina, San Diego, California, U.S.A.). In another embodiment, and without limitation, a microbiome gene analysis may include identifying a de novo assembly comprising a 5,325,941 bp length, wherein the de novo assembly may contain a GC content of 50.59%. In an embodiment and without limitation, a microbiome gene analysis may identify 114 RNA coding sequences, 82 tRNAs, 11 ncRNAs, 260 pseudogenes, and/or 2 CRISPR arrays of a microbiota genome.

Still referring to FIG. 1, computing device 104 initiates a sequence identification function 120. As used in this disclosure, a “sequence identification function” is a function and/or algorithm that identifies a genomic sequence and/or target sequence of a proposed therapeutic oligomer. Computing device 104 may initiate sequence identification function 120 as a function of identifying a plurality of prospective gene targets 124 as a function of the genomic library. As used in this disclosure, a “prospective gene target” is a gene of interest that is encoded in a chromosome, plasmid, DNA, RNA, dsRNA, dsDNA, mRNA, siRNA, tRNA, hpRNA, and/or the like thereof that may be suitable to be innervated with an oligomer. In an embodiment, and without limitation, identifying a plurality of prospective gene targets 124 as a function of the genomic library may include determining a prospective gene target 124 for a given target pathogen, such as a viral agent and/or genomic sequence. In an embodiment, prospective gene targets 124 may correspond to proteins that are essential for growth and/or inhibit the viral agent and/or pathogen mechanism. As used in this disclosure, the terms “inhibit” and/or “inhibition” means to reduce a molecule, a reaction, an interaction, a gene, an mRNA, and/or a protein's expression, stability, function, and/or activity by a measurable amount, and/or to prevent such entirely. In an embodiment, and without limitation, inhibitors may include compounds with binding affinity, such as but not limited to an antagonist, to partially and/or totally block stimulation, to decrease, prevent, or delay activation, to inactivate, desensitize, and/or downregulate a protein, a gene, and/or an mRNA, or otherwise to modulate the expression, function, and/or activity thereof. For instance, and without limitation, inhibition may include, be included in, and/or involve translational and/or transcriptional downregulation as described in further detail below. A person of ordinary skill in the art would be aware that any of the DNA and/or mRNA sequences described above can be targeted by antisense inhibitors. In another embodiment, and without limitation, target sequences may include sequences present in one or more agents associated to a microbiome disease, immune disease, oncological disease, protein disorder, neurodegenerative disease, infectious disease, and/or the like thereof. In an embodiment, and without limitation, inhibition may include inhibiting an inflammatory response. In another embodiment, and without limitation, prospective gene targets 124 may correspond to proteins that are essential for growth and/or inhibit the SARS-COV-2. For example, and without limitation, prospective gene target 124 may correspond to a protein, DNA, RNA, genomic sequence, and the like thereof associated to SARS-COV-2, such that an inhibition may occur. In another embodiment, and without limitation, prospective gene targets 124 may correspond to proteins that are essential for growth and/or inhibit one or more viral agents, pathogens, and the like thereof. In an embodiment, and without limitation, target sequences may include those of a homologous gene or mRNA sequence in a viral agent such as influenza. In another embodiment, and without limitation, target sequences may include those of E. coli and/or a homologous gene or mRNA sequence in another target bacterium. Given the benefit of this disclosure, those of ordinary skill in the art will be able to identify a target sequence and/or design an antisense inhibitor oligomer to target the gene or mRNA sequence. Target sites on DNA and/or RNA (e.g., sRNA) associated with antibiotic resistance may include any site to which binding of an antisense oligomer may inhibit the function of the DNA or RNA sequence. Inhibition may be caused by steric interference resulting from an antisense oligomer binding the DNA and/or RNA sequence such that a prevention of proper transcription of the DNA sequence and/or translation of the RNA sequence occurs.

Still referring to FIG. 1, computing device 104 may identify a plurality of prospective gene targets 124 as a function of a PNA Finder toolbox. As used in this disclosure, a “PNA Finder toolbox” is a tool that provides the user with a list of candidate PNA sequences as well as several selection criteria that can be used to predict the efficacy of a given candidate. In an embodiment, and without limitation, PNA Finder toolbox may allow a user to filter the list of prospective PNA candidates and synthesize/identify the most promising sequence. The prospective PNA with the most promising sequence may then be quickly subjected to efficacy testing in bacterial cultures, microbiome disease cultures, immune disease cultures, oncological disease cultures, protein disorder cultures, neurodegenerative disease cultures, infectious disease cultures, and/or the like thereof. The resultant data may be used to both select the optimal PNA antibiotic, antiviral, and/or therapeutic and to inform and improve upon the PNA Finder selection process, wherein efficacy testing is described below in detail. In an embodiment, PNA Finder toolbox may be built using Python 2.7. Additionally, in order to run on a Windows operating system, the toolbox may incorporate the program Cygwin to provide a Unix-like environment. The user interface for the PNA Finder Toolbox may also be constructed using the Python 2.7 package Tkinter, version 8.5.

Still referring to FIG. 1, computing device 104 generates proposed therapeutic oligomer sequence 108 as a function of prospective gene targets 124 and an oligomeric machine-learning model 128. As used in this disclosure, an “oligomeric machine-learning model” is a machine-learning model configured to generate a proposed therapeutic oligomer output given prospective gene targets 124 as inputs. A machine-learning model incorporates a machine-learning process. A “machine-learning process”, as used herein, is a process that automatically uses a body of data known as “training data” and/or a “training set” to generate an algorithm that will be performed by a computing device/module to produce outputs given data provided as inputs. This is in contrast to a non-machine learning software program where the commands to be executed are determined in advance by a user and written in a programming language. Oligomeric machine-learning model 128 may include one or more oligomeric machine-learning processes such as supervised, unsupervised, or reinforcement machine-learning processes that computing device and/or a remote device may or may not use in the determination of proposed therapeutic oligomer. As used in this disclosure, “remote device” is an external device to computing device. Oligomeric machine-learning process may include, without limitation machine-learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naive bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

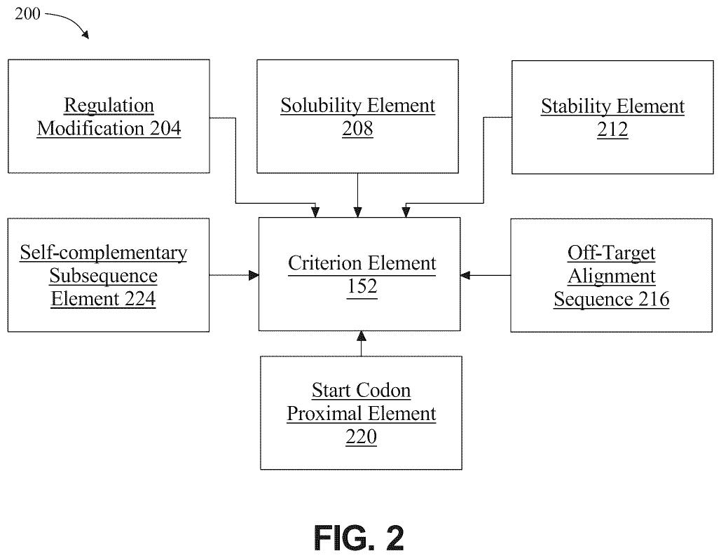

Still referring to FIG. 1, oligomeric machine-learning model 128 is trained as a function of an oligomeric training set. As used in this disclosure, an “oligomeric training set” is a training set that correlates a plurality of prospective gene targets 124 and/or oligomers that regulate gene expression to a proposed therapeutic oligomer sequence. As used herein, “gene expression” is a process that synthesizes a functional gene product from a gene. Additional details will be described below, in reference to FIG. 2. For example, and without limitation, a prospective gene target of G-CSF and an oligomer that inhibits gene expression of acute radiation syndrome (ARS) may relate to proposed therapeutic oligomer sequence 108 of PNA initiators and/or PNA activators. The oligomeric training set may be received as a function of user-entered valuations of prospective gene targets, oligomers that regulate gene expression, and/or proposed therapeutic oligomer sequences. Computing device 104 may receive oligomeric training set by receiving correlations of prospective gene targets 124 and/or oligomers that regulate gene expression that were previously received and/or identified during a previous iteration of generating a proposed therapeutic oligomer sequence. The oligomeric training set may be received by one or more remote devices that correlate a prospective gene target and/or oligomer that regulates gene expression to a proposed therapeutic oligomer sequence. The oligomeric training set may be received in the form of one or more user-entered correlations of prospective gene targets and/or oligomers that regulate gene expression to proposed therapeutic oligomer sequences.

Still referring to FIG. 1, computing device 104 may receive oligomeric machine-learning model 128 from a remote device that utilizes one or more oligomeric machine-learning processes, wherein a remote device is described above in detail. For example, and without limitation, a remote device may include a computing device, external device, processor, and the like thereof. A remote device may perform an oligomeric machine-learning process using oligomeric training set to generate proposed therapeutic oligomer sequence and transmit the output to computing device. A remote device may transmit a signal, bit, datum, or parameter to computing device that at least relates to proposed therapeutic oligomer sequence. Additionally, or alternatively, a remote device may provide an updated machine-learning model. For example, and without limitation, an updated machine-learning model may be comprised of a firmware update, a software update, an oligomeric machine-learning process correction, and the like thereof. As a nonlimiting example a software update may incorporate a new prospective gene target that relates to an oligomer that regulates a gene expression. Additionally, or alternatively, updated machine-learning model may be transmitted to the remote device, wherein the remote device may replace the oligomeric machine-learning model with the updated machine-learning model and generate proposed therapeutic oligomer sequence 108 as a function of prospective gene target using the updated machine-learning model. The updated machine-learning model may be transmitted by remote device and received by computing device as a software update, firmware update, or corrected oligomeric machine-learning model. For example, and without limitation, oligomeric machine-learning model 128 may utilize a random forest machine-learning process, wherein the updated machine-learning model may incorporate a gradient boosting machine-learning process.

Still referring to FIG. 1, computing device 104 may generate proposed therapeutic oligomer sequence 108 as a function of a classifier. A “classifier”, as used in this disclosure, is a machine-learning model generated by a machine-learning algorithm known as a “classification algorithm”, that sorts inputs into categories or bins of data, outputting the categories or bins of data and/or labels associated therewith. A classifier may include a mathematical model, neural net, or program, among others. Additional details regarding classification algorithm will be described in further detail below. A classifier may be configured to output at least a datum that labels or otherwise identifies a set of data that are clustered together, found to be close under a distance metric as described below, or the like. Computing device and/or another device may generate a classifier using a classification algorithm, whereby a computing device derives a classifier from training data. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers.

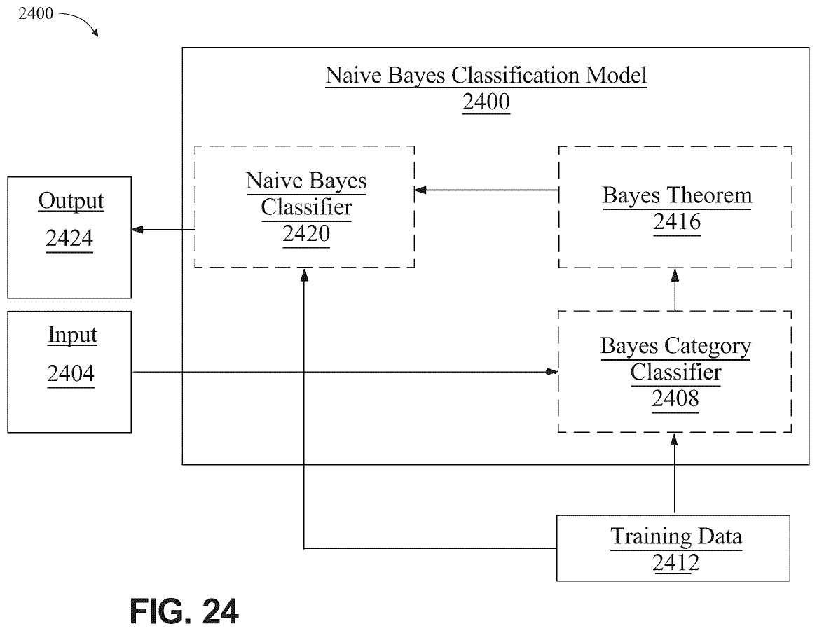

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)×P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naive Bayes algorithm may be generated by first transforming training data into a frequency table. Computing device 104 may then calculate a likelihood table by calculating probabilities of different data entries and classification labels. Computing device 104 may utilize a naive Bayes equation to calculate a posterior probability for each class. A class containing the highest posterior probability is the outcome of prediction. Naïve Bayes classification algorithm may include a Gaussian model that follows a normal distribution. Naïve Bayes classification algorithm may include a multinomial model that is used for discrete counts. Naïve Bayes classification algorithm may include a Bernoulli model that may be utilized when vectors are binary. A Naïve Bayes Classification Model is disclosed further with reference to FIG. 24.

With continued reference to FIG. 1, computing device 104 may be configured to generate a classifier using a K-nearest neighbors (KNN) algorithm. A “K-nearest neighbors algorithm”, as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors' algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors' algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a nonlimiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized”, or divided by a “length” attribute, such as a length attribute/as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

In an embodiment, and still referring to FIG. generating the proposed therapeutic oligomer may include employing a PNA Finder, which may include a toolbox that comprises two primary functions: a “Get Sequences” tool for finding an initial list of PNA candidates and/or a “Find Off-Targets” tool for determining incidental undesired alignments of those candidates, wherein a “Find Off-Targets” tool is described below. In an embodiment, and without limitation, PNA Finder may include oligomeric machine-learning model as described above. In another embodiment, and without limitation, each of these functions may contain several sub-functions to aid in toolbox workflow, efficiency, and/or an in silico PNA screening process. These tools may be designed to function as a cohesive workflow, starting from a user-provided list of gene targets and providing a filtered set of stable and highly specific PNA candidates that represent the most viable therapeutic options. The generalized toolbox, as described herein, may also include a graphical user interface to ensure that it is a streamlined process, as well as to avoid difficulties with the command line interface on which several of its constitutive programs operate. In an embodiment, “Get Sequences” may be used for identifying an initial list of PNA candidates and performing a preliminary screening of these candidates. Additionally, or alternatively, PNA Finder may generate proposed therapeutic oligomer to activate and/or inhibit expression of genes as a function of gene targets for the human genome. In another embodiment, and without limitation, PNA Finder may generate proposed therapeutic oligomer to for one or more pathogens, viral agents, and the like thereof. In an embodiment, and without limitation, PNA Finder may generate proposed therapeutic oligomer sequence 108 as a function of a criteria for design of species specific PNAs, which may include without limitation: (i) whether a gene target is involved in radiation response, (ii) whether a TIR and/or IRES sequence is amenable to design of peptide PNAs with low melting temperature when targeting mRNA (expression inhibition) or upstream promoter regions (−150, −116, −78, and −7 positions) when targeting DNA (transcriptional activation), and (iii) whether possible off-target sites within a human transcriptome and between microbiome species are not present. Additionally, or alternatively, criteria for design of species specific PNAs may include: (i) whether a gene target is essential, (ii) whether there is evidence that gene silencing of target and/or inhibition of cognate protein is growth inhibitory, (iii) whether a TIR and RBS sequence is amenable to design of peptide PNAs with low melting temperature, (iv) whether possible off-target sites within and between species are absent from TIR and RBS sites, (v) whether homologues are present in a desired number of species for targeting multiple strains, and (vi) whether the TIR of an mRNA has at least two base pairs between species when designing unique PNAs.

Still referring to FIG. 1, computing device is configured to output a sequence warning for proposed therapeutic oligomer sequence 108. As used in this disclosure, a “sequence warning” is a notification and/or signal that identifies a solubility and/or self-complementation issue. In an embodiment, and without limitation, a sequence warning may analyze proposed therapeutic oligomer sequence and denote possible solubility issues. For example, and without limitation, solubility issues may denote that a proposed therapeutic oligomer sequence has an uncommon common-ion effect, ionic strength element, solubility equilibrium, temperature, and/or the like thereof, making it difficult to dissolve and/or suspend in a solvent. In another embodiment, and without limitation, sequence warning may analyze proposed therapeutic oligomer sequence and denote possible self-complementation issues. For example, and without limitation, self-complementation issues may denote that a proposed therapeutic oligomer sequence has more than six bases that may form self-complementary subsequences.

Still referring to FIG. 1, computing device 104 identifies a genomic locus 132 that the proposed therapeutic oligomer is predicted to bond to. As used in this disclosure, a “genomic locus” is a fixed position on a chromosome, RNA chain, and/or DNA chain where a particular gene and/or genetic marker is located. For example, and without limitation, genomic locus 132 may denote that a particular gene and/or genetic marker is located on a p-arm of a chromosome. As a further nonlimiting example, genomic locus 132 may denote that a particular gene and/or genetic marker is located on a q-arm of a chromosome. As used in this disclosure, “bonding” is a process of forming a connection between two or more molecules, ions, and/or atoms that are non-associated. In an embodiment, and without limitation, bonding may include forming a connection as a function of an intermolecular force that causes two or more molecules to be attracted and/or repulsed by each other. For example, and without limitation, bonding may include connecting two or more molecules, ions, and/or atoms as a function of a dipole-dipole interaction. As a further nonlimiting example, bonding may include connecting two or more molecules, ions, and/or atoms as a function of a hydrogen bond. As a further nonlimiting example, bonding may include connecting two or more molecules, ions, and/or atoms as a function of a London dispersion force. As a further nonlimiting example, bonding may include connecting two or more molecules, ions, and/or atoms as a function of a cation-pi interaction. Computing device 104 identifies genomic locus 132 that the proposed therapeutic oligomer is predicted to bond to as a function of an off-target sequence function 136. As used in this disclosure, an “off-target sequence function” is an algorithm and/or model that determines the number of inaccuracies and/or off-targets of a target sequence. A “target sequence”, as used herein, is a nucleotide sequence that is complementary to an antisense molecule. A target sequence may include a DNA sequence or an mRNA sequence. An antisense molecule may include an antisense PNA. In an embodiment, and without limitation, off-target sequence function 136 may include a function that reduces the expected number of off-targets in a genome, which is given by the following equation, under the simplifying assumption of total randomness of the genome:

$${E}_{off - targets} = \frac{1}{{4}^{N}} \times ( genome size )$$

wherein E_{off-targets} is the expected number of off-targets, genome size is the number of base pairs in the genome, and N is the length of proposed therapeutic oligomer sequence 108, such as but not limited to the length of a PNA.

Still referring to FIG. 1, genomic locus 132 is identified as a function of identifying an incidental alignment 140 as a function of proposed therapeutic oligomer sequence 108 and genomic library 112. As used in this disclosure, an “incidental alignment” is an undesired alignment of proposed therapeutic oligomer sequence. For example, and without limitation, incidental alignment 140 may denote that an undesired alignment of a PNA may occur as a function of an extraneous hydrogen bond formation. As a further nonlimiting example, incidental alignment 140 may denote that an undesired alignment of a PNA may occur due to a poor solubility and/or strong London dispersion forces.

Still referring to FIG. 1, genomic locus 132 is identified as a function of modeling incidental alignment 140 to a corresponding genome assembly location 144. As used in this disclosure, a “genome assembly location” is a sequence composition of a portion of a genome that resides in a location of a chromosome. For example, and without limitation, a first sequence of a genome may reside in a p-arm of a chromosome, wherein incidental alignment 140 is modeled to the first sequence of the genome located in a p-arm of the chromosome. In an embodiment, and without limitation, modeling may include producing a 3D computer model and/or virtual representation of a proposed therapeutic oligomer sequence 108 and/or genome assembly location 144. For example, and without limitation, computing device 104 may generate a topographical and/or 3D rendering of proposed therapeutic oligomer sequence 108 and/or genome assembly location 144. In an embodiment, and without limitation, 3D rendering may include a solid model of proposed therapeutic oligomer sequence 108 and/or genome assembly location 144. As used in this disclosure, a “solid model” is a computer model of three-dimensional solids. For example, and without limitation, solid model may include one or more geometric and/or solid models of proposed therapeutic oligomer sequence 108 and/or genome assembly location 144. In an embodiment, and without limitation, solid model may include a solid representation scheme, such as a primitive instancing, spatial occupancy enumeration, cell decomposition, boundary representation, surface mesh modeling, sweeping, constructive solid geometry, implicit representation, parametric and/or feature-based modeling, and the like thereof. In an embodiment, and without limitation, solid model may incorporate one or more voxels, polygonal meshes, parametric shapes, and the like thereof to represent proposed therapeutic oligomer sequence 108 and/or genome assembly location 144.

In an embodiment, and still referring to FIG. 1, modeling incidental alignment 140 to a corresponding genome assembly location 144 may include producing a seed length as a function of a length parameter associated to proposed therapeutic oligomer sequence 108. As used in this disclosure, a “seed length” is the length of a proposed therapeutic oligomer sequence and/or PNA length. As used in this disclosure, a “length parameter” is the total distance of each bond and/or oligomer that comprises proposed therapeutic oligomer sequence and/or PNA. For example, and without limitation, length parameter may be a total distance expressed in units such as angstroms, nanometers, micrometers, and/or the like thereof. In an embodiment, and without limitation, the default for proposed therapeutic oligomer sequence 108 and/or PNA length may be set to 12, based on competing factors. In another embodiment, and without limitation, increasing proposed therapeutic oligomer sequence 108 and/or PNA length may reduce expression regulation as a function of an increased proposed therapeutic oligomer sequence and/or PNA length. In another embodiment, and without limitation, increasing proposed therapeutic oligomer sequence 108 and/or PNA length may enhance binding strength and/or specificity. For example, and without limitation, a PNA length of 12 may yield sufficient inhibition and/or may yield less than one expected off-target for even the largest genomes. In one exemplary embodiment, a default 12-mer PNA may be designed to complement a base pattern such as ***** AUG ****. This default positioning may be located close to a start codon and may have a strong inhibitory effect in prokaryotes. The window may be expanded according to a user's requirements to produce multiple candidate sequences of a given length. For instance, and without limitation, a window of (−6, −4) with a default 12-mer PNA would produce three PNA candidates, complementary to each of the following base patterns: ****** AUG ***, **** AUG ****, **** AUG**

In an embodiment, and without limitation, modeling incidental alignment 140 may include aligning proposed therapeutic oligomer sequence 108 to a corresponding genome assembly location 144 as a function of a seed length. Such alignment may include orienting proposed therapeutic oligomer sequence 108 in a three-dimensional space as a function of a binding affinity between the proposed therapeutic oligomer sequence 108 and genome assembly location 144. Additionally, or alternatively, modeling incidental alignment 140 may include modeling incidental alignment 140 as a function of an alignment and a user-specified number of allowed mismatches. A “mismatch”, as used herein, is an improper base pair bonding of a nucleobase. For example, and without limitation, a mismatch may include a guanine nucleobase paired with a thymine nucleobase. As a further nonlimiting example, a mismatch may include an adenine nucleobase paired with a cytosine nucleobase. In an embodiment, and without limitation, mismatch may be due to nucleobase tautomerization. In another embodiment, and without limitation, modeling may include producing a 3D computer model and/or virtual representation, as described above. For example, and without limitation, modeling may include producing a 3D computer model of an alignment and a user-specific number of allowed mismatches. In an embodiment, and without limitation, the default number of allowed mismatches may be set to zero.

Still referring to FIG. 1, identifying genomic locus 132 includes identifying genomic locus 132 as a function of incidental alignment 140 model and an overlap element 148. As used in this disclosure, an “overlap element” is an element of data that denotes a proposed therapeutic oligomer sequence 108 overlapping with one or more genomic features of genomic library 112. For example, and without limitation, overlap element 148 may denote those one or more sequences of proposed therapeutic oligomer sequence 108 that overlap with a plurality of genomic features of genomic library 112. In an embodiment, and without limitation, overlap element 148 may include a BAM file that may be used as an input for a BEDTools “window” function, wherein the BEDTools “window” function may be used to identify whether a particular PNA-genome alignment in the BAM file overlaps with any genomic features, as identified by an input GFF genome annotation file. In another embodiment, a “Find Off-Targets” tool may then examine the BED file output of “window” function to determine which proposed therapeutic oligomer sequences 108 and/or PNAs are expected to have off-target alignments in coding sequences, as well as which of these coding sequence alignments occur near to a start codon, as described below. The gene coordinate inputs may be used to define the region around a start codon where inhibition is expected. The default for “Find Off-Targets” tool may be set to (−20, 20). In an embodiment, and without limitation, “Find Off-Targets” tool may be used to search for incidental alignments between a model and/or list of PNA target sequences and a genome assembly. In an embodiment, and without limitation, “Find Off-Targets” tool may take an input from a PNA target sequences, a genome assembly, and/or a corresponding genome annotation file. In another embodiment, “Find Off-Targets” tool may incorporate one or more of the following parameters: the number of allowed alignment mismatches, PNA sequence length, and/or a pair of gene coordinates relative to a +1 translation start site. In an embodiment, “Find Off-Targets” tool may provide an output comprising the total off-target counts for each proposed therapeutic oligomer sequence and/or PNA.

In an embodiment, and still referring to FIG. 1, identifying genomic locus 132 may further include outputting a first file representing a potentially inhibitory alignment of proposed therapeutic oligomer sequence 108. As used in this disclosure, a “potentially” inhibitory alignment is an inhibitory alignment with a probabilistic outcome that is expressed on a probability interval [0,1], wherein exceeding a threshold denotes a likelihood of the outcome occurring. In an embodiment, probability may be defined according to a characteristic function, which may include, without limitation, a step function having output values on a probability interval such as [0,1] or the like; step function may have an output representing 100% or probability of 1 for values falling in a range and zero for a representation of zero probability for values not in the range. For example, and without limitation, “Find Off-Targets” tool may produce as an output a BED file of all potentially inhibitory PNA alignments, wherein a BED file may be written with the features corresponding to each ID, and a “Get Sequences” tool may print output to indicate any matches. As a further nonlimiting example, “Find Off Targets” tool may edit the coordinates of a BED file according to the PNA sequence length and/or the gene coordinates parameters. In an embodiment, and without limitation, “Find Off-Targets” tool may include an off-target counts option, wherein the option totals the number of potentially inhibitory off-targets for each PNA and may provide those sums in a separate file. Off-target predictions may be used as another means of screening PNA candidates, either to avoid targeting other genes within a target genome or to avoid targeting another organism altogether. This function is especially valuable in PNA antibiotic design, as it allows for the design of highly specific antisense PNAs that may avoid broad antibiotic action against a microbiome environment.

In an embodiment, and still referring to FIG. 1, identifying genomic locus 132 further comprises outputting a second file identifying a potentially off-target alignment of proposed therapeutic oligomer sequence 108. For example, and without limitation, the coordinates and/or strand designation of each gene target in the original BED file may be used to create a new BED file, wherein each set of genomic coordinates corresponds to the locus that each respective proposed therapeutic oligomer sequence and/or PNA may target. A BEDTools function “getfasta” may then be used to produce a FASTA file of the PNA target sequences from these BED file coordinates and the input genome assembly FASTA file. An output file with the PNA sequences-reverse complements of the target sequences—may also be produced. If the options for sequence warnings and STRING database analysis are selected, these elements may be included in the output file as well. The STRING database analysis may provide a network of experimentally verified, computationally predicted, and inferred protein interactions for each gene target, as well as the number of total connections between the genes of this network.

Still referring to FIG. 1, proposed therapeutic oligomer sequence 108 is selected as a function of sequence identification function 120, genomic locus 132, and a criterion element 152. Criterion element 152 is described below and may include a regulation modification, solubility element, stability element, presence of a self-complementary subsequence, presence of an off-target alignment sequence, start codon proximal element of a gene target, and the like thereof. In an embodiment, and without limitation, proposed therapeutic oligomer sequence 108 and/or PNA may be selected for synthesis according to application-specific needs for sequence stability and specificity. In an embodiment, and without limitation, application-specific needs may be ascertained from an output of the PNA Finder toolbox.

In an embodiment, and still referring to FIG. 1, proposed therapeutic oligomer sequence 108 may be selected as a function of treating an acute radiation syndrome (ARS) and/or radiation toxicity to inhibit and/or activate expression of radiation responsive gene. As used herein, acute radiation syndrome (ARS) and/or radiation toxicity is an acute illness caused by radiation of part of or whole body by a high dose (>1Gray or Gy) of radiation for a short period of time. Accordingly, therapeutic oligomer sequence 108 may be selected to treat such radiation related pathologies. In this embodiment, rationally designed PNAs that may inhibit and/or activate expression of radiation-responsive genes, such as G-CSF, 2) GM-CSF, 3) EPO), and 4) GG, may allow prevention or reduction of radiation-induced conditions, such as Acute Radiation Syndrome (ARS). In this embodiment, at least two types of therapeutic oligomer sequences and/or PNAs may be selected, specifically, inhibitors and activators, wherein inhibitors may include without limitation single-stranded antisense PNAs designed to bond to mRNAs of a targeted gene to block its translation. Activators may include, without limitation, antigene PNAs designed to bond to genomic DNA in the upstream promoter regions of a targeted gene to increase its expression. As used in this disclosure, an “antigene” is a synthetic molecule designed to specifically bind to a particular DNA sequence and modulate its transcriptional activity. Antigens are typically short, single-stranded oligonucleotides that can bind to complementary sequences within the DNA double helix, thereby blocking the transcription machinery from accessing a target gene. Such binding may effectively silence the expression of a gene, making antigens a powerful tool in gene regulation and therapeutic applications. Antigens are used in molecular biology and therapeutic research to downregulate or silence specific genes involved in disease processes, such as cancer or genetic disorders. By preventing the expression of harmful genes, antigens offer a targeted approach to treatment, potentially reducing side effects compared to more traditional therapies.

In an embodiment, and still referring to FIG. 1, a PNA finder may be utilized to design a catalog of unique PNA inhibitor and/or activator molecules against radiation-responsive genes and reduce chances of off-target effects by comparison to gut microbiome and human transcriptome.

In another embodiment, and still referring to FIG. 1, computing device 104 may design a PNA molecule for activation/inhibition of genes involved in radiation response. For example, and without limitation, computing device may design of approximately three to five 20-mer PNA molecules that target a translational start site (TIS) and/or internal ribosome entry site (IRES) of the mRNA encoded by G-CSF, GM-CSF, EPO and GG genes, with the target sequence in the middle of an oligomer and 3-5 nucleotides flanking the target region. In this embodiment, a cell penetrating peptide (CPP) and/or quantum dot (QD) at the N terminus of a target PNA may be attached to the PNA for increased cellular entry. Additionally, or alternatively, a highly positively charged protein transduction domain of transactivator of transcription (TAT) sequence (YGRJJRRQRRR) (SEQ ID NO: 8) from HIV-1 may be incorporated to successfully facilitate PNA delivery into mammalian cells and nucleus via an energy- and receptor-independent mechanism called micropinocytosis.

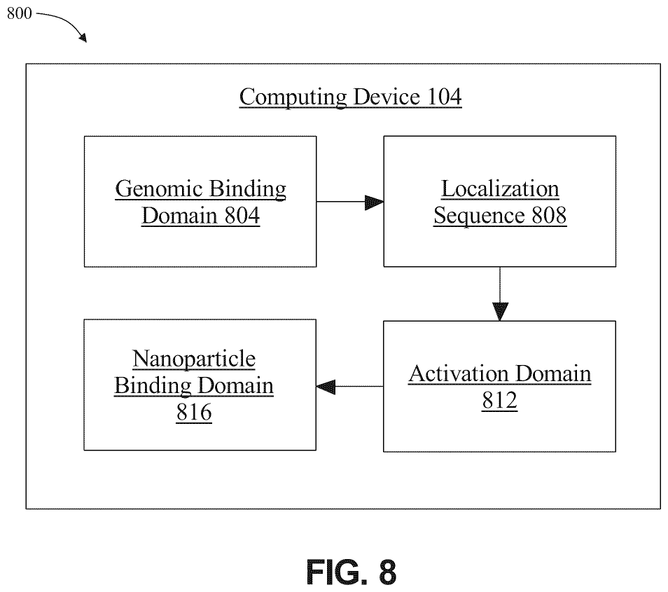

In another embodiment, and still referring to FIG. 1, computing device 104 may design a PNA activators as a function of a modular approach, which may comprise: 1) a sequence-specific DNA binding domain (DBD) configured to direct a PNA to an appropriate promoter, and 2) an amino acid sequence motif that may act as an activation domain (AD) to recruit transcription complexes to a gene target promoter region, a modular approach of which is described below in reference to FIG. 8. For example, and without limitation, such PNA driven activation may generate a nearly 8-fold activation of expression of gamma-globin gene using a chimeric VP2 minimal ADPNA-TAT in mouse bone marrow cells and human primary peripheral blood cells compared to basal expression. VP2 minimal AD is a highly acidic 16 amino acid sequence (MLGDFDLDMLGDFDLD) (SEQ ID NO: 9) derived from the herpes simplex virus C terminus transactivation domain of VP16. This artificial AD has been shown to be highly effective in vitro when linked to DNA-binding domains. In another embodiment a chimeric PNA sequence to bond to the promoter of G-CSF, GM-CSF, EPO and GG genes may be selected by designing 15 mer PNA centered at −150, −116, −78, and −7 positions relative to the transcriptional start sites of the gene. In order to facilitate binding to DNA, a Lysine residue may be attached to 3′ to give a PNA molecule a positive change to enhance strand invasion. For activating gene expression during PNA synthesis, additionally, a chimeric PNA-VP2 binding domain-binding peptide chimera capable of activating transcription may be designed. Finally, for enhancing intracellular delivery, a CPP may be attached based on a TAT sequence or quantum dot, as described below.

In another embodiment, and still referring to FIG. 1, computing device 104 may design a design of PNAs for species-centered strategy. For example, and without limitation, PNA molecules may be designed to prevent translation of one or more essential genes within a pathogenic organism. As a further nonlimiting example, and without limitation, PNA molecules may be designed to prevent translation of one or more essential genes within a pathogenic organism, such as SARS-COV-2, HIV, influenza, and the like thereof. In this embodiment, computing device may design a 12-mer PNA molecule that targets the translational start site (TIS) or ribosome binding site (RBS) of the mRNA encoded by an essential gene. Such 12-mer long PNAs may be designed against genes in pathogens using a stepwise targeting method, such that antisense oligomers are designed with the target sequence in the middle of the oligomer and 3-5 nucleotides flanking the target region.

In another embodiment, and still referring to FIG. 1, computing device 104 may design 12-mer PNAs oligomers, such as but not limited to α-RBS and α-STC against a ribosome binding site (RBS) and/or a start codon (STC) of TEM-1 β-lactamase (bla) mRNA to prevent the ribosomal binding and ribosomal migration respectively, both causing inhibition of translation of bla transcript to prevent the production of active β-lactamase enzyme. The 12-mers may be conjugated, to a positively charged (KFF) 3K CPP. In an embodiment, and without limitation, both α-RBS and α-STC may exhibit no off-target activity. In another embodiment, and without limitation, computing device 104 may design PNA molecules that target six essential genes including, but not limited to, folC, which is involved in metabolism, ffh which is involved in cell signaling, lexA, which is a key regulator of stress response, and fnrS, which is a small Hfq binding RNA, rpsD, which is involved in protein biosynthesis, and gyrB, which is involved in DNA replication.

Still referring to FIG. 1, computing device 104 synthesizes a therapeutic oligomer 156 as a function of proposed therapeutic oligomer sequence 108. As used in this disclosure, a “therapeutic oligomer” is a polymer comprising at least a repeating unit that produces a therapeutic effect as a function of regulating an expression of one or more genes and/or polynucleotides. In an embodiment, therapeutic oligomer 156 may include a nanoligomer, as described above. As used in this disclosure, “synthesizing” is a step of initiating a manufacturing process and/or automated synthesis process that builds one or more therapeutic oligomers. In some embodiments, a manufacturing process is a process used to form a product, which may be an end-product, or a part used to assemble an end-product, by the performance of one or more manufacturing steps. One or more steps in the manufacturing process may include physical modifications to a product and/or programming and modeling steps used to perform the modifications, such as modeling the product, computing toolpaths, and/or other algorithms for the product's manufacture.

In an embodiment, and still referring to FIG. 1, synthesizing therapeutic oligomer 156 may include an additive manufacturing device. An “additive manufacturing device”, as used in this disclosure, is a device that performs additive manufacturing processes. As used in this disclosure, an “additive manufacturing process” is a process in which material is added incrementally to a body of material in a series of two or more successive steps. A material may be added in the form of a stack of incremental layers; each layer may represent a cross-section of an object to be formed upon completion of an additive manufacturing process. Each cross-section may, as a nonlimiting example, be modeled on a computing device as a cross-section of graphical representation of the object to be formed; for instance, a computer-aided design (CAD) tool may be used to receive or generate a three-dimensional model of an object to be formed, and a computerized process, such as a “slicer” or similar process, may derive from that model a series of cross-sectional layers that, when deposited during an additive manufacturing process, together will form the object. Steps performed by an additive manufacturing system to deposit each layer may be guided by a computer-aided manufacturing (CAM) tool. Persons skilled in the art will be aware of many alternative tools and/or modeling processes that may be used to prepare a therapeutic oligomer, including without limitation the peptide synthesis, synthetic reactions, and the like thereof.

In an embodiment, and still referring to FIG. 1, therapeutic oligomers 156 may comprise PNAs, wherein PNAs are described above. In an embodiment, and without limitation, PNAs may inhibit gene expression in a target host. A “target host”, as used herein, is an organism and/or entity that PNA is interacting with. In an embodiment, and without limitation, an organism and/or entity may include a pathogen, viral agent, and the like thereof. In another embodiment, and without limitation, PNAs may upregulate gene expression in a target host. In one or more embodiments, target host may include specific organism, organ, tissue, cell type, or even subcellular compartment where nanoparticle should accumulate and deliver its payload, as opposed to all other non-target sites. Additionally, or alternatively, computing device 104 may synthesize therapeutic oligomer 156 and/or PNA using a solid-state PNA synthesis including fluorenylmethyloxycarbonyl (Fmoc) chemistry. As used in this disclosure, a “solid-state PNA synthesis” is a synthetic reaction that utilizes one or more solid supports for physical stability to build an oligomer. For example, and without limitation, solid supports may include a resin. Resin may include any resin that is physically stable and/or permits a rapid filtration of liquids. In an embodiment, and without limitation, resin may include one or more gel-type support resins, surface-type support resins, and/or composite resins. In another embodiment, and without limitation, resin may be able to withstand repeated use of trifluoroacetic acid (TFA). In another embodiment, resin may include one or more resins as a function of a desired product such as, but not limited to, a C-terminal carboxylic acid and/or an amide. In an embodiment, and without limitation, resin may include a Wang resin. Additionally, or alternatively, therapeutic oligomer 156 and/or PNA may be synthesized using tert-butyloxycarbonyl(BOC or tBoc) chemistry. Therapeutic oligomers 156, PNAs, and/or other polynucleotides may also be chemically derivatized using methods recognized by those of ordinary skill in the art, with the benefit of this disclosure. For example, PNAs may have amino and carboxy groups at the 5′ and 3′ ends, respectively, that can be further derivatized.

Still referring to FIG. 1, synthesizing therapeutic oligomer 156 may include coupling proposed therapeutic oligomer sequence 108 to a nanostructure. As used in this disclosure, a “nanostructure” is a structure of intermediate size between microscopic and molecular structures. For example, and without limitation, nanostructure may include a structure of a size in the range of 0.1 nm to 100 nanometers. In an embodiment, and without limitation, nanostructures may include spherical nanoparticles. As used in this disclosure, a “nanoparticle” is a three-dimensional object existing on a nanoscale, wherein the particle is between 0.1 nm and 100 nm in each spatial dimension. For example, and without limitation, a nanoparticle may include a spherical nanoparticle with a diameter of 23 nm. In an embodiment, and without limitation, a nanoparticle may include a transition metal nanoparticle. As used in this disclosure, a “transition metal nanoparticle” is a nanoparticle composed of a transition metal. “Transition metal,” as used in this disclosure, is an element in d-block (group 3-12) of a periodic table. In one or more embodiments, transition metal may be characterized by having partially filled d-orbitals. In one or more embodiments, partially filled d-orbitals may allow transition metal to form multiple ions with varying charges. For example and without limitation, d-block elements of a periodic table may include scandium, titanium, vanadium, chromium, manganese, iron, cobalt, nickel, copper, zinc; yttrium, zirconium, niobium, molybdenum, technetium, ruthenium, rhodium, palladium, silver, cadmium; hafnium, tantalum, tungsten, rhenium, osmium, iridium, platinum, gold, mercury; and the superheavy congeners rutherfordium, dubnium, seaborgium, bohrium, hassium, meitnerium, darmstadtium, roentgenium, and copernicium. In one or more embodiments, transition metal may be used in transition metal nanoparticle. In one or more embodiments, transition metal may function as a transition metal core in a transition metal nanoparticle. For example, and without limitation, a transition metal nanoparticle may include a gold nanoparticle. As a further nonlimiting example, a transition metal nanoparticle may include a copper nanoparticle. As a further nonlimiting example, a transition metal nanoparticle may include a zinc nanoparticle. In an embodiment, and without limitation, a transition metal nanoparticle may include one or more transition-metal elements from groups 3-12 of the periodic table of elements. In an embodiment, and without limitation, coupling proposed therapeutic oligomer sequence 108 to nanostructure may include forming a covalent bond. As used in this disclosure, a “covalent bond” is a chemical bond that involves sharing of electrons between atoms. Covalent bond may include electron pairs that are shared and/or localized between two atoms, as a function of a stable balance of attractive and/or repulsive forces. In an embodiment, and without limitation, covalent bond may allow molecules and/or atoms to fill one or more valence shells of an atom to produce a stable electron configuration. In another embodiment, covalent bond may include one or more interactions such as, but not limited to o-bonding, x-bonding, metal-to-metal bonding, agnostic interactions, bent bonds, three-center two-electron bonds, three-center four-electron bonds, delocalized bonds, and the like thereof.

In an embodiment and still referring to FIG. 1, coupling proposed therapeutic oligomer sequence 108 to a nanostructure may include utilizing a chemical synthesis. As used in this disclosure, a “chemical synthesis” is a physical process of mixing reagents and/or solvents to produce a product using one or more chemical reactions. For example, and without limitation, chemical synthesis may include mixing one or more nanostructures, proposed therapeutic oligomer sequences, and/or solvents to produce therapeutic oligomer 156. As used in this disclosure, a “reagent” is a substance and/or mixture to be consumed in a chemical reaction. For example, and without limitation, reagent may include a chemical, reactant, and the like thereof. In an embodiment, reagent is placed in a receptacle. As used in this disclosure, a “receptacle” is an object and/or space that is used to confine a plurality of reagents. For example, and without limitation, receptacle may include one or more beakers, flasks, bottles, jars, test tubes, desiccators, glass evaporating dishes, watch glasses, petri-dishes, slides, graduated cylinders, volumetric flasks, burettes, ebulliometers, condensers, retorts, drying pistols, and the like thereof. In an embodiment, and without limitation, receptacle may be placed in or coupled to a modulation component. As used in this disclosure, a “modulation component” is a structure and/or object that regulates one or more external properties of the receptacle. For example, and without limitation, a modulation component may regulate one or more temperatures as a function of a water bath, oil bath, sand bath, ice bath, hot plate, Bunsen burner, flame, meeker burner, and the like thereof. As a further nonlimiting example, a modulation component may regulate one or more pressures as a function of a vacuum system, compressor, and the like thereof. As a further nonlimiting example, a modulation component may regulate one or more wavelengths as a function of a polarization and/or slit system. In an embodiment, and without limitation, a modulation component may include a stirring apparatus. As used in this disclosure, a “stirring apparatus” is a device that employs a rotating magnetic field beneath a receptacle. In an embodiment, and without limitation, a stirring apparatus may be incorporated in one or more modulation components. For example, and without limitation, a magnetic stir bar may be placed inside of receptacle, wherein a stirring apparatus may rotate a magnetic field beneath receptacle such that the magnetic stir bar immersed in the reagents is forced to spin and/or rotate at a given angular velocity, which may be described in units such as without limitation revolutions per minute (rpm). In an embodiment, and without limitation, a magnetic stir bar may include a bar-shaped octagonal and/or circular rod.

Still referring to FIG. 1, a reagent may be added into receptacle using a transfer device. As used in this disclosure, a “transfer device” is a device and/or tool that transports a volume of liquid. In an embodiment, and without limitation, a transfer device may include one or more media dispensers. In another embodiment, and without limitation, a transfer device may include a pipette. As used in this disclosure, a “pipette” is a device and/or tool that creates a vacuum displacement to draw up a liquid, wherein releasing the vacuum dispenses the liquid. For example, and without limitation, a pipette may include one or more air displacement micropipettes, electronic pipettes, positive displacement pipettes, volumetric pipettes, graduate pipettes, Pasteur pipettes, transfer pipettes, pipetting syringes, Van Slyke pipettes, Ostwald-Folin pipettes, glass micropipettes, microfluidic pipettes, low volume pipettes, and the like thereof. In an embodiment, and without limitation, a pipette may create a vacuum displacement above a receptacle, wherein a pipette tip is located within reagent and/or solvent located within the receptacle. As used in this disclosure, a “pipette tip” is a tapered cylindrical tube that has a first aperture with a first diameter and a second aperture with a second diameter, wherein the second diameter is greater than the first diameter. In an embodiment, and without limitation, a first aperture may be configured to draw a reagent, liquid, and/or solvent. In another embodiment, and without limitation, a second aperture may be configured to be secured to a pipette and/or transfer device. Additionally, or alternatively, a transfer device may incorporate one or more valves, microfluidic channels, stopcocks, and the like thereof to control the transfer of one or more reagents, liquids, and/or solvents.

In an embodiment, and still referring to FIG. 1, synthesizing therapeutic oligomer 156 may include synthesizing therapeutic oligomer 156 using an automated synthesizer 160. As used in this disclosure, an “automated synthesizer” is a device and/or apparatus that automatically performs one or more synthetic processes. In an embodiment, and without limitation, automated synthesizer 160 may include or be included in computing device 104. In another embodiment, and without limitation, automated synthesizer 160 may include or be included in a remote device, pipettor, robotic device, and the like thereof. In an embodiment, and without limitation, automated synthesizer 160 may perform one or more synthetic processes such as, but not limited to, a chemical synthesis, a peptide synthesis, a coupling process, and/or the like thereof. For example, and without limitation, automated synthesizer 160 may include an Apex 396 peptide synthesizer (AAPPTec, LLC, Louisville, KY, U.S.A). In an embodiment, and without limitation, automated synthesizer 160 may be used to perform a solid-state PNA synthesis using Fmoc chemistry, as described above, on MBHA rink amide resin, at a 30 μmol scale, wherein Fmoc-PNA monomers may be include A, C, and G monomers that are protected at amines with benzyloxycarbonyl(Bhoc) groups. In another embodiment, and without limitation, automated synthesizer 160 may synthesize therapeutic oligomer 156 with a cell-penetrating peptide, such as (KFF) 3K, which may have lysine residues protected with Boc groups. In another embodiment, and without limitation, automated synthesizer 160 may be used to perform solid-phase Fmoc chemistry at a 10 μmol scale on MBHA rink amide resin, wherein Fmoc-PNA monomers may include A, C, and G monomers protected at amines with Bhoc groups. In another embodiment, and without limitation, automated synthesizer 160 may synthesize therapeutic oligomer 156 with a N-terminal cell-penetrating peptide (KFF) K., which may have lysine residues protected with Boc groups. In another embodiment, automated synthesizer 160 may incorporate one or more modes of operation, such as but not limited to a semi-automated mode and/or a fully automated mode to synthesize therapeutic oligomer 156 and/or PNA. For example, and without limitation, a semi-automated mode may allow a user to input one or more reagents, wherein automated synthesizer 160 automatically mixes and/or modulates a chemical synthesis. As a further nonlimiting example, a fully automated mode may allow a user to select a therapeutic oligomer and/or PNA from a graphical user interface, on a display, wherein automated synthesizer 160 measures the reagents, transfers the reagents to a receptacle, and performs a chemical synthesis without user intervention.

Still referring to FIG. 1, synthesizing therapeutic oligomer 156 may include synthesizing a therapeutically effective amount. As used in this disclosure, a “therapeutically effective amount” is an amount of a therapeutic oligomer that will relieve to some extent one or more of the symptoms of the ailment, infection, and/or disorder being treated. In an embodiment, and without limitation, a therapeutically effective amount may include an amount of therapeutic oligomer 156 that has the effect of (1) reducing the pathogen, (2) inhibiting (that is, slowing to some extent, preferably stopping) pathogen and/or viral agent growth, (3) inhibiting (that is, slowing to some extent, preferably stopping) pathogenicity, and/or (4) relieving to some extent (or, preferably, eliminating) one or more signs or symptoms associated with the pathogen and/or viral agent. In another embodiment, and without limitation, for treatment of a viral agent such as SARS-COV-2, a therapy such as SARS-COV-2, a therapeutically effective amount may include an amount of therapeutic oligomer 156 that has the effect of (1) reducing the viral agent magnitude, (2) inhibiting (that is, slowing to some extent, preferably stopping) viral growth, (3) inhibiting (that is, slowing to some extent, preferably stopping) viral pathogenicity, and/or (4) relieving to some extent (or, preferably, eliminating) one or more signs or symptoms associated with the viral agent. In another embodiment, a therapeutically effective amount may include an amount of therapeutic oligomer 156 that treats one or more ailments, infections, diseases, and/or disorders. As used in this disclosure, “treating” is a process of reversing, alleviating, inhibiting the progress of, or preventing a disorder or condition to which such term applies, or one or more symptoms of such disorder or condition. For example, and without limitation, treating a viral agent associated with influenza may include alleviating one or more symptoms and/or proliferations of the viral agent present in an organism.

Still referring to FIG. 1, synthesizing therapeutic oligomer 156 may further include clarifying therapeutic oligomer 156. As used in this disclosure, “clarifying” is a process of purification and/or separation that extracts one or more distinct therapeutic oligomers 156 from a chemical synthesis. For example, and without limitation, clarifying may include purifying therapeutic oligomer 156 from a chemical synthesis such that only one therapeutic oligomer remains. In an embodiment, and without limitation, clarifying therapeutic oligomer 156 may be performed using a filter. As used in this disclosure, a “filter” is a physical and/or chemical medium that isolates and/or extracts one or more therapeutic oligomers 156 of interest. In an embodiment, and without limitation, a filter may include a size exclusion filter. As used in this disclosure, a “size exclusion filter” is a filter including a plurality of apertures with a diameter that allows substances and/or liquid capable of fitting within the diameter to flow across the filter. In an embodiment, and without limitation, a size exclusion filter may be uniform and/or non-uniform. For example, and without limitation, a size exclusion filter may be comprised of a plurality of 50 μm apertures. As a further nonlimiting example, a size exclusion filter may be comprised of a plurality of apertures ranging from 5-500 μm. In another embodiment, and without limitation, a filter may include an electromagnetic filter. As used in this disclosure, an “electromagnetic filter” is a filter that attracts one or more metallic particles and/or similar electromagnetically responsive structures by applying an electromagnetic field, thereby removing them from a chemical synthesis. For example, and without limitation, electromagnetic filter may include one or more magnets that attract a nanostructure and/or nanoparticle to remove them from a chemical synthesis.

In an embodiment, and still referring to FIG. 1, therapeutic oligomer 156 and/or PNA may be purified using a liquid chromatography component. In an embodiment, and without limitation, a “liquid chromatography component” is a component capable of separating therapeutic oligomers 156 from a chemical synthesis. In an embodiment, and without limitation, a liquid chromatography component may include a component that separates analytes using a stationary phase and a mobile phase. For example, and without limitation, a stationary phase may include a phase including a porous solid such as but not limited to glass, silica, alumina, free silanol, bonded silanol, geminal silanol, siloxane, and/or the like thereof. As a further nonlimiting example, a mobile phase may include a phase including a liquid solvent such as but not limited to water, acetonitrile, chloroform, isopropyl alcohol, ethanol, hexane, butane, propane, benzene, and/or the like thereof. In an embodiment, and without limitation, a physical separation component may separate analytes and/or biological samples as a function of a capacity factor, k′. As used in this disclosure, a “capacity factor” is a measurable value representing the strength of the interaction between an analyte and/or biological sample with a stationary phase as it flows through the mobile phase. Capacity factor may be determined by:

k′=tᵣ−tₘ/tₘ

where t, is the retention time of the analyte and tm is the retention time of a reference compound. A “reference compound”, as used herein, is a compound that has a known retention time and/or does not interact with the stationary phase. As used in this disclosure, a “retention time” is a time period that it takes for a compound to travel through liquid chromatography component. A time period may be measured in units such as seconds, minutes, hours, days, and/or the like thereof. In an embodiment, and without limitation, a physical separation component may separate analytes and/or biological samples as a function of a selectivity factor, a. As used in this disclosure, a “selectivity factor” is a measurable value associated with an amount of separation between two or more therapeutic oligomers 156. Selectivity factor may be determined by:

α=k′₂/k′₁=tᵣ_{₂}−tₘ/tᵣ_{₁}−tₘ

where tᵣ_{₁} is the retention time of a first therapeutic oligomer, tᵣ_{₂} is the retention time of a second therapeutic oligomer, k′₁ is the capacity factor of the first therapeutic oligomer, and k′₂ is the capacity factor of the second therapeutic oligomer. For example, and without limitation, a selectivity factor for liquid chromatography component may be 2.15 for an amount of separation between a first trifluoroacetic acid salt PNA and a second trifluoroacetic acid salt PNA, when both of which are dissolved in an acetonitrile mobile phase and interacting with a C18 stationary phase.

In an embodiment, and still referring to FIG. 1, synthesizing therapeutic oligomer 156 may include precipitating the therapeutic oligomer 156. As used in this disclosure, “precipitating” is a chemical process of transforming a dissolved substance into an insoluble solid. In an embodiment, and without limitation, precipitating therapeutic oligomer 156 may include transforming a dissolved therapeutic oligomer 156 in a solvent into an insoluble solid by super-saturating a solvent. As a nonlimiting example, precipitating therapeutic oligomer 156 may be performed using diethyl ether. Additionally, or alternatively, synthesizing therapeutic oligomer 156 may include drying the therapeutic oligomer 156. As used in this disclosure, “drying” is a process of removing solvent and/or liquid from a solid. For example, and without limitation, drying may be performed over a time period, such as four or less days to achieve a higher purity of therapeutic oligomer 156, such as but not limited to a 90% purity. In an embodiment, automated synthesizer 160 may synthesize PNA products by precipitating and/or purifying products using trifluoroacetic acid salts.

In an embodiment, and still referring to FIG. 1, synthesized therapeutic oligomers 156 may be purified and further tested in an in vitro and/or in vivo environment for specific activity. For example, and without limitation, therapeutic oligomer 156 may be evaluated as a function of an up- and/or down-regulation of the expression of one or more gene targets. As a further nonlimiting example, therapeutic oligomer 156 may be evaluated as a function of potentiating known therapeutic compounds, such as but not limited to potentiating activity of traditional small-molecule antibiotics.

In an embodiment, and still referring to FIG. 1, automated synthesizer 160 may be configured to perform an automated parallel high-throughput in-lab synthesis capable of producing a plurality of therapeutic oligomers 156 and/or PNAs per run in a short period of time, such as less than a day. In this embodiment, therapeutic oligomers 156 may be synthesized using a standard solid-phase manual or automated peptide synthesis, using either Boc/tBoc- or Fmoc-protected PNA monomers. For example, and without limitation, for a PNA-CPP sequence of N-terminal-KFFKFFKFFK (SEQ ID NO: 10)-AEEA (linker, SEQ ID NO: 2)-10 CACCGGCAAGTG-C terminal (SEQ ID NO: 11), firstly, a CPP peptide portion (KFF) 3K of the PNA-CPP conjugate may be synthesized on a peptide synthesizer, under a normal automatic mode, using a Fmoc-D-Lys (Boc) Wang resin (110 mg, 0.51 mmol/g). This may be followed by therapeutic oligomer synthesis using Fmoc-protected PNA monomers with exocyclic amino acid groups of A, T, G and C using a single-shot delivery feature.

The synthesis of PNA may be started on Fmoc-D-Lys (Boc)-Wang resin (50 mg, 0.78 mmol/g). The Fmoc protecting group may be removed by using 20% piperidine in dimethylformamide (DMF) twice for 5 min each. This may be followed by a download of resin, which may be followed by a partial coupling to free amino acid groups. Unreacted free amino acids may be capped by adding a PNA-capping solution (2 mL, for 5 min) containing 5% N,N-Diisopropylethylamine (DIEA). Resin may further be washed and dried. The downloading may be measured in a UV spectrophotometer at 290 nm. The downloaded resin may be kept in the automated synthesizer, and the coupling (0.5 mL of each PNA monomer, 0.3 mL hexafluorophosphate azabenzotriazole tetramethyl uronium (HATU), and 0.3 mL of 196.3 mM DIEA), washing (with DMF, MeOH, and DCM), deprotection, and washing steps may be repeated automatically in a continuous way until an exemplary 12-mer PNA product is obtained although, as noted elsewhere, PNAs of different sizes may be obtained. The final products of PNA-CPP may be purified with semi-preparative HPLC using a C-18 column, as described above, and characterized using NMR and/or a mass analyzer component. As used in this disclosure, a “mass analyzer component” is a component capable of analyzing a mass-to-charge ratio of one or more ionic fragments generated by therapeutic oligomer 156 and/or PNA. In an embodiment, and without limitation, a mass analyzer component may include a linear quadrupole. As used in this disclosure, a “linear quadrupole” is a mass analyzer that filters ions as a function of four metal rods that create a quadrupolar electric field. Such quadrupolar electric field may allow ions of specific mass-to-charge ratios to be guided along the central axis of the four parallel arranged rods, while eliminating other mass-to-charge ratios. In an embodiment, and without limitation, the four metal rods may be hyperbolic which may match the electric field that is produced. In an embodiment, and without limitation, quadrupolar electric field may be generated by the four rods through a series of tunable RF and DC voltages. In an embodiment, and without limitation, linear quadrupole may allow for specific mass-to-charge ratios to be selected and/or a range of mass-to-charge ratios to be selected to allow for either an entire mass window to be collected and/or peak hopping. As used herein, “peak hopping”, is an analysis of a specific peak and/or mass-to-charge ratio to be identified. In another embodiment, and without limitation, a mass analyzer component may include a time-of-flight mass analyzer. As used in this disclosure, a “time-of-flight mass analyzer” is a mass analyzer that separates ions over time across a field-free drift space. As used in this disclosure, a “field-free drift space” is an enclosed space wherein a limited or no electric field interacts with the ions present in the enclosed space. In an embodiment, and without limitation, ions may be focused into an ion packet. As used in this disclosure, an “ion packet” is a group and/or cluster of ions. An ion packet may be pulsed into a field-free drift space with a uniform amount of kinetic energy. Such uniform kinetic energy provided to the ion packet may allow smaller ions to have higher velocities compared to larger ions. As a result, smaller ions will reach a detector faster due to their higher velocities, while the larger ions will reach the detector slower, due to their lower velocities. In an embodiment, and without limitation, a mass analyzer component may include a tandem mass spectrometer component. As used in this disclosure, a “tandem mass spectrometer component” is a component capable of elucidating structural data of an ion based on its distribution of mass-to-charge ratios. For example, and without limitation, a tandem mass spectrometer component may fragment one or more ions of interest to produce a fragmented charged ion and a neutral loss. As used in this disclosure, a “fragmented charged ion” is an ion that lacks at least an atom of the parent ion, wherein a parent ion is the first ion present in a mass analyzer component. For example, and without limitation, fragmented charged ion may include a daughter ion and/or ion having a direct relationship to its parent ion. As used in this disclosure, a “neutral loss” is a neutral fragment including an atom or molecule that is expelled from a parent ion. In an embodiment, and without limitation, tandem mass spectrometer component may elucidate structural data as a function of an ion activation method such as but not limited to collision-induced dissociated, surface induced dissociation, electron transfer dissociation, in-source decay, post-source decay, photodissociation, and/or the like thereof.

Still referring to FIG. 1, synthesizing therapeutic oligomer 156 may further include analyzing the therapeutic oligomer 156 as a function of a validation protocol. As used in this disclosure, a “validation protocol” is a protocol and/or method that identifies an efficacy and/or toxicity of therapeutic oligomer 156. In an embodiment, and without limitation, a validation protocol may include utilizing normalized growth data to determine the most effective PNA, as well as to improve the efficacy predictions of the PNA Finder toolbox. For example, and without limitation, a moderate correlation between a STRING database protein network node degree-a measure of the connectivity of a given gene within viral agent metabolism- and normalized 16-hour growth data of viral agents, may be utilized to identify an efficacy prediction. In an embodiment, and without limitation, a validation protocol may measure inhibition of each PNA against that of a scrambled nonsense sequence. In an embodiment, and without limitation, a validation protocol may incorporate one or more in vitro, in vivo, and/or macrophage-based host-infection models to identify one or more efficacies and/or toxicities therapeutic oligomer 156. In an embodiment, and without limitation, validation protocol may include one or more PNA interaction assays. For example, and without limitation, PNA interaction assays may include three colonies picked from a plate and used to inoculate three separate overnight cultures in 1 mL Cation Adjusted Mueller Hinton broth (CAMHB) each. After 16 hours, the culture may be diluted 1:10,000 in a 384-well microplate using three biological replicates per condition. The total culture volume for each 15 treatments may be 50 μL. PNA may be stored at −20° C., dissolved in 5% v/v DMSO in water. Growth in the plate may be monitored at an absorbance of 590 nm every 20 minutes for 24 hours, with shaking between measurements. In another embodiment, and without limitation, PNA interaction may include clinical isolates that may be obtained and grown in CAMHB at 37° C. with 225 rpm shaking or on solid CAMHB with 1.5% agar at 37° C. Clinical isolates may be maintained as freezer stocks in 90% CAMHB, in 10% glycerol at −80° C. Freezer stocks may be streaked out onto solid CAMHB and incubated for 16 hours to produce single colonies prior to experiments. For each biological replicate, a single colony may be picked from solid media and grown for 16 hours in liquid CAMHB prior to experiments. At the start of experiment, each culture may be diluted 1:10,000 in fresh CAMHB and added to either a control experiment without PNA or a 10 μM PNA sample. PNA samples may be stored in 5% DMSO to aid in stability. Interaction effects may be evaluated for significance using a two-way ANOVA test, and S values may be calculated with respect to an expected growth inhibition, as calculated by a Bliss Independence model. The S-value for a given timepoint may be calculated as follows:

$$S = ( \frac{{OD}_{AB}}{{OD}_{0}} ) ( \frac{{OD}_{PNA}}{{OD}_{0}} ) - ( \frac{{OD}_{AB , PNA}}{{OD}_{0}} )$$

In an embodiment, and without limitation, for a given timepoint, the variable OD_{AB} represents optical density with only carbapenem treatment, OD₀ represents the optical density without treatment, OD_{PNA} represents optical density with only antisense-PNA treatment, and OD_{AB,PNA} represents the optical density with a combination treatment. Error bars for S-values may be calculated by propagating standard error values for each term. In an embodiment, and without limitation, interaction effects may be represented as heatmaps and/or dendrograms. Additionally, or alternatively, a Euclidean distance metric, optimal leaf ordering, and/or average linkage function may be used to identify interaction effects.

Still referring to FIG. 1, validation protocol may include testing therapeutic oligomer 156 in a high-throughput host infection model. For example, and without limitation, PNAs may undergo in vitro screening in broth cultures. Cultures of each individual pathogen and/or viral gent may be grown in broth or other appropriate media. PNA molecules may be designed for each strain to either target them individually or in combinatorial manner. Scrambled PNA sequence may be used as control. PNAs may be supplied in a range of concentrations (0-50 μM) to various combinations of cultures for a period of 24 hours. The number of viable cells remaining at the end of this time point may be measured using colony forming unit analysis. The dominant strains in a culture may be identified by sampling the liquid culture at the end of experiment and measuring a relative distribution of the strains using pathogen specific primers in a quantitative polymerase chain reaction (PCR) assay. As used in this disclosure, a “polymerase chain reaction” is an instrument that amplifies deoxyribonucleic acid (DNA) samples. In an embodiment, and without limitation, polymerase chain reaction may amplify a small quantity of DNA sample to a large quantity such that an analysis may be performed. In another embodiment, and without limitation, a polymerase chain reaction may include a thermal cycling element. As used in this disclosure, a “thermal cycling element” is an element that exposes a chemical to repeated cycles of heating and cooling. In an embodiment, and without limitation, thermal cycling element may allow for DNA melting, enzyme-driven DNA replication, and the like thereof. In an embodiment, and without limitation, polymerase chain reaction may include a primer. As used in this disclosure, a “primer” is a single-stranded nucleic acid used by living organisms in the initiation of DNA synthesis. In an embodiment, and without limitation, a primer may include an oligonucleotide, as described above, that is a complementary sequence to a target DNA region. Additionally, or alternatively, a polymerase chain reaction may include a DNA polymerase. As used int this disclosure, a “DNA polymerase” is an enzyme that catalyzes the synthesis of DNA molecules from molecular precursors of DNA. In an embodiment, and without limitation, DNA polymerase may create two identical DNA duplexes from a single original DNA duplex. In another embodiment, and without limitation, DNA polymerase may create a nucleotide to a three prime (3′)-end of a DNA strand. In an embodiment, and without limitation, DNA polymerase may include a heat-stable DNA polymerase. As used in this disclosure, a “heat-stable DNA polymerase” is an enzyme capable of catalyzing DNA synthesis at high temperatures. For example, and without limitation, heat-stable DNA polymerase may include a Taq polymerase enzyme. In an embodiment and without limitation, a polymerase chain reaction component may be configured to perform DNA cloning, gene cloning, gene manipulation, gene mutagenesis, construction of DNA-based phylogenies, diagnosis of genetic disorders, monitoring of genetic disorders, amplification of DNA, analysis of DNA genetic fingerprints, detection of pathogens in nucleic acid tests, and the like thereof. Additionally, or alternatively, validation protocol may measure one or more efficacies and/or toxicities as a function of a high-throughput fluorescent infection assay that may quantify the effectiveness of select PNAs.

Still referring to FIG. 1, synthesizing therapeutic oligomer 156 may further include updating genomic library 112 as a function of the therapeutic oligomer 156 and a genomic outcome. As used in this disclosure, a “genomic outcome” is one or more gene expression modifications and/or physiological responses to therapeutic oligomer 156. In an embodiment, and without limitation, genomic outcome may be determined as a function of profiling a gene expression in response to a PNA. For example, and without limitation, gene expression may be profiled as a function of a differential expression in outer membrane porin operon (omp) genes, previously linked to carbapenem-resistance, and resistance-related genes. As a further nonlimiting example, ompF may be found to be significantly differentially expressed in any condition with respect to no treatment (underexpressed in meropenem, 30 minutes), wherein ompA and ompC expression tracked closely with the no-treatment conditions in all experiments. Additionally, or alternatively, expression levels of ertapenem and meropenem experiments may be directly compared at each time point, wherein none of the three genes were found to be significantly differentially expressed, and wherein no resistance-related genes were differentially expressed in any condition. In an embodiment, and without limitation, a gene expression may be profiled in response to an ertapenem and/or meropenem treatment. For example, viral agent influenza A may be exposed to ertapenem and/or meropenem, and a gene expression profile may be examined after a period of time, which may be measured in units such as seconds, minutes, hours, days, and the like thereof. Viral agent influenza A may be diluted 1:20 from overnight cultures and grown for 1 hour to exponential phase prior to treatment with 2 μg/mL of ertapenem or 1 μg/mL of meropenem. In an embodiment, genomic expression may be monitored as a function of comparing the RNA sequencing data from ertapenem- and meropenem-treated samples to an untreated control at the same timepoint. General expression trends may be evaluated using hierarchical clustering across genes and conditions. Conditions may be found to cluster by timepoint which may suggest a generalized and transient response. For example, and without limitation, 41 transcripts that were DE in both treatments after 30 minutes of exposure, six transcripts DE in both antibiotics after 60 minutes of exposure, and six that were DE in both treatments at 30 and 60 minutes may be evaluated as a function of a hierarchical clustering. In an embodiment, and without limitation, genes such as flhC and flhD may encode components of transcriptional regulator FlhDC, which may be responsible for regulating motility-associated functions such as swarming and flagellum biosynthesis. Both flhC and flhD genes may be significantly underexpressed at 30 and 60 minutes.

Still referring to FIG. 1, synthesizing therapeutic oligomer 156 may include incorporating therapeutic oligomer 156 into a vector delivery system. As used in this disclosure, a “vector delivery system” is a delivery vehicle that aids in delivering one or more therapeutic oligomers to a target host. In an embodiment, and without limitation, a vector delivery system may include one or more probiotic microorganisms. For example, and without limitation, probiotic microorganisms that may act as a delivery vehicle for one or more PNAs include yeasts such as Saccharomyces, Debaromyces, Candida, Pichia, and Torulopsis, molds such as Aspergillus, Rhizopus, Mucor, Penicillium, and Torulopsis, and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, and Lactobacillus. Specific examples of suitable probiotic microorganisms are: Saccharomyces cerevisiae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus. In another embodiment, vector delivery system may include one or more bacteria based “Micro-Robots” using Type III bacterial secretion systems. For example, and without limitation, bacteria based “Micro-Robots” may repurpose bacterial secretion systems, such as Type III (T3SS) or Type IV secretion systems, to deliver PNAs. As a further nonlimiting example, bacteria based “Micro-Robots” may include T3SS, which are molecular machines used by many Gram-negative bacterial pathogens including pathogens Shigella, Yersinia, Salmonella, and Pseudomonas, to inject proteins, known as effectors, directly into eukaryotic host cells. In an embodiment, and without limitation, bacteria based “Micro-Robots” may include proteins that manipulate host signal transduction pathways and cellular processes to a pathogen's advantage. Additionally, or alternatively, synthesizing therapeutic oligomer 156 may include administering a vector delivery system and/or therapeutic oligomer 156 to an organism, user, subject, and/or the like thereof.

In an embodiment, and still referring to FIG. 1, bacteria based “Micro-Robots” may repurpose the intrinsic Type III secretion system in Gram-negative bacteria, such as Salmonella, wherein the Gram-negative bacteria may uptake and deliver PNAs to a target eukaryotic cell. In another embodiment, a T3SS function may be introduced in a non-pathogenic strain of bacteria, such as E. Coli Nissle 1917, a probiotic strain that is easily culturable and has been tested in humans for treatment of irritable bowel syndrome. In this embodiment, a T3SS from a pathogen such as Shigella flexneri may be incorporated into a synthetic biology-based approach where such a protein delivery system may be composed of two parts: (i) a ~31-kb long minimal DNA sequence that contains operons required for a functional T3SS from Shigella flexneri, and (ii) the transcriptional activator VirB to induce expression of the T3SS. In another embodiment, bacteria based “Micro-Robots” may include a kill switch circuit under the control of the Ipac promoter to activate cell lysis once E. coli enters mammalian cells. As used herein, Ipac is a native Shigella T3SS-encoded translocator protein that is activated once Shigella invades a mammalian cell. This may address both bio-safety concerns that E. coli Nissle 1917 should be killed once it has entered the mammalian cell, as well as result in an efficient secretion of PNA-CPP molecules. In this embodiment, a kill switch design is based on an expression of a holin and/or antiholin. Holin is a protein that forms pores in cell membranes. Anti-holin forms a dimer with holin, which is no longer active. Once pores are formed by holin, lysozyme may access the periplasmic space and degrade the cell wall, causing cell lysis.

In another embodiment, and still referring to FIG. 1, vector delivery system may include a nanoparticle-based delivery system. As used in this disclosure, a “nanoparticle-based delivery system” is a delivery vehicle including one or more nanoparticles, consistent with details described above. For example, and without limitation, a nanoparticle delivery system may provide a delivery vehicle for enhanced PNA transport, lowered toxicity, and increased bioavailability. In an embodiment, and without limitation, nanoparticle-based delivery system may include a gold nanoparticle delivery system. In an embodiment, and without limitation, a nanoparticle-based delivery system may include a lipid nanoparticle (LNP), liposome, nanoliposome, nano-lipid sphere, transfersome, noisome, ethosome, nanovesicle, and/or the like thereof. Additionally, or alternatively, therapeutic oligomers 156 may be introduced to mammalian cells that have been exposed to radiation, such as gamma-radiation. In this embodiment, the inventors may use a high-throughput screening method for PNA molecules using human macrophages, hematopoietic stem cells exposed to gamma-radiation, and in some instances microgravity to better simulate conditions in space. The target PNAs may be tested to demonstrate gene-specificity, reduced radiation response, increased transport, the lowered toxicity.

In an embodiment, and still referring to FIG. 1, therapeutic oligomers 156 and/or PNAs may be designed and/or synthesized to allow for PNA antisense inhibition of RNA sequencing targets. In an embodiment, system 100 may be configured for designing and/or synthesizing antisense PNA structures. For example, and without limitation, designing antisense PNA may include using transcriptomic data to generate a list of gene targets, which, together with a whole-genome assembly and genome annotation, may be used as inputs for a FAST tool PNA Finder, wherein the tool may be used to design multiple antisense PNA candidates for each gene target, with 12-mer sequences-a length that seeks to optimize both specificity and transmembrane transport—that were complementary to mRNA nucleotide sequences surrounding a translation start codon. PNA Finder may then filter this set of candidates to minimize the number of predicted off-targets within the pathogen genome, to maximize solubility, and to avoid any self-complementing sequences. For a FAST Build module, a single PNA for each gene target may be selected and synthesized using Fmoc chemistry, consistent with details described above. These PNAs may then be tested in cultures in combination with carbapenem to determine whether the two treatments would interact as predicted. A two-way ANOVA test may be used to assess interaction significance, and a normalized S-value may be used to compare an observed growth to an expected growth, as predicted by a Bliss Independence Model for drug combinations. Normalized S-value is described above in detail.

In an embodiment, and still referring to FIG. 1, therapeutic oligomer 156 may be multiplexed. As used in this disclosure, “multiplexing” is a process of incorporating two or more therapeutic oligomers 156 to achieve a therapeutic effect. For example, and without limitation, multiplexing may include multiplexing two or more therapeutic oligomers 156 such that CSF-2 may be upregulated, IL-10 may be upregulated, and/or TNF-α may be downregulated. In an embodiment, and without limitation, multiplexing two or more therapeutic oligomers 156 including CSF-2 upregulators and/or IL-10 upregulators may reduce an expression of proinflammatory cytokines and/or increase IL-10 expression. In an embodiment, and without limitation, multiplexing may allow for therapeutic profiling of immune engineering therapeutics.

Now referring to FIG. 2, an exemplary embodiment 200 of implementing criterion element 152 is illustrated. As used in this disclosure, a “criterion element” is an element of data denoting one or more principles and/or standards that pertain to therapeutic oligomer sequence 108. In an embodiment, and without limitation, criterion element 152 may be identified as a function of a screening and/or analysis of previously synthesized therapeutic oligomers 156, wherein analysis and/or screening is described above. In another embodiment, and without limitation, criterion element 152 may be identified as a function a chemical property database. As used in this disclosure, a “chemical property database” is a database and/or datastore of chemical properties of molecules and/or oligomers. In an embodiment, and without limitation, chemical property database may include a structure element, safety element, molecular formula, molecular weight, toxicity element, physical description, color, form, odor, taste, boiling point, melting point, density, vapor pressure, octanol-water partition coefficient (LogP), viscosity, corrosivity, heat of vaporization, surface tension, refractive index, polarity, dipole moment, and the like thereof. For example, and without limitation, criterion element 152 may include a regulation modification 204. As used in this disclosure, a “regulation modification” is an effect and/or influence that a proposed therapeutic oligomer sequence 108 may have on a gene target expression. As used in this disclosure, “gene expression” is a process that synthesizes a functional gene product from a gene. For example, and without limitation, gene expression may include producing end products such as, but not limited to, proteins and/or non-coding RNA structures. For example, and without limitation, regulation modification 204 may denote an inhibition of a gene target expression. As a further nonlimiting example, regulation modification 204 may denote an upregulation of a gene target expression. In an embodiment, and without limitation, regulation modification 204 may denote that GM-CSF and/or CSF2 may be upregulated to increase protein expression. In another embodiment, and without limitation, regulation modification 204 may upregulate one or more associated G-CSF, growth factors, and/or a significant number of proinflammatory cytokines, such as but not limited to IL-1α, IL-1β, TNF-α, TNF receptors, IL-10, NLRP1, IL-6, TNFR1, NF-κB, erythropoietin (EPO), and the like thereof. Additionally, or alternatively, regulation modification 204 may denote an upregulation of hemopoietic proteins and/or proinflammatory enzymes. As a further nonlimiting example, regulation modification 204 may denote that a proposed therapeutic oligomer sequence may have no effect and/or influence on a gene target expression.

Still referring to FIG. 2, criterion element 152 may include a solubility element 208. As used in this disclosure, a “solubility element” is a chemical property of a solvent to dissolve a proposed therapeutic oligomer sequence. For example, and without limitation, solubility property of proposed therapeutic oligomer sequences may be diverse as a function of a common-ion effect, ionic strength element, solubility equilibrium, temperature, and the like thereof. In an embodiment, and without limitation, solubility properties may be variable as a function of the solvent. For example, and without limitation, a solvent element for a proposed therapeutic oligomer sequence may vary as a function of a solvent of gastric acid in comparison to blood. Criterion element 152 may include a stability element 212. As used in this disclosure, a “stability element” is a measurable value denoting the magnitude of reactivity of a proposed therapeutic oligomer sequence. For example, and without limitation, stability element 212 may denote that proposed therapeutic oligomer sequence has a high stability as a function of a low Gibbs Free Energy. As a further nonlimiting example, stability element 212 may denote that proposed therapeutic oligomer sequence has a low stability as a function of a high Gibbs Free Energy.

Still referring to FIG. 2, criterion element 152 may include an off-target alignment sequence 216. As used in this disclosure an “off-target alignment sequence” is an element of data denoting the number of alignments predicted to inhibit a gene and/or translation. In an embodiment, and without limitation, off-target alignment sequence 216 may be aligned using Clustal X version 2.1 to (i) its own genome, (ii) across desired number of genomes, (iii) across human transcriptome and genome (for any potential side-effects). Genomic analysis of possible binding sites may also be conducted in Artemis using a cut-off of 2 base pair mismatches. Only PNA sequences that uniquely target pathogens and/or viral agents of interest may be considered. For example, in an embodiment, unique PNAs will be designed against known gene sequences obtained from the genome library. Additionally, or alternatively, criterion element 152 may include a start codon proximal element 220. As used in this disclosure, a “start codon proximal element” is an element of data denoting the proximity of the genomic locus to the start codon of the genomic sequence, wherein the proximity may inhibit a gene and/or translation. For example, and without limitation, start codon proximal element 220 may include a default range that identifies an alignment as a function of being set to (−20, 20), wherein a minor translation inhibition may occur at 17 bases upstream of a beta-lactamase start codon, and wherein no significant translation inhibition may occur 23 bases downstream of the same start codon. In an embodiment, start codon proximal element 220 may vary from gene to gene. Additionally, or alternatively, criterion element 152 may include a self-complementary subsequence element 224. As used in this disclosure, a “self-complementary subsequence element” is an element of data denoting the presence of one or more complementary binding sequences in a gene strand that may fold, bend, and/or turn to create a double-stranded structure internally. In an embodiment, and without limitation, self-complementary subsequence element 224 may denote the presence of six or more bases that are self-complementary and/or may bond to each other without the presence of an external substrate such as DNA. As a further nonlimiting example, self-complementary subsequence element 224 may denote one or more hairpin structures and/or orientations that may enhance a self-complementary subsequence binding affinity.

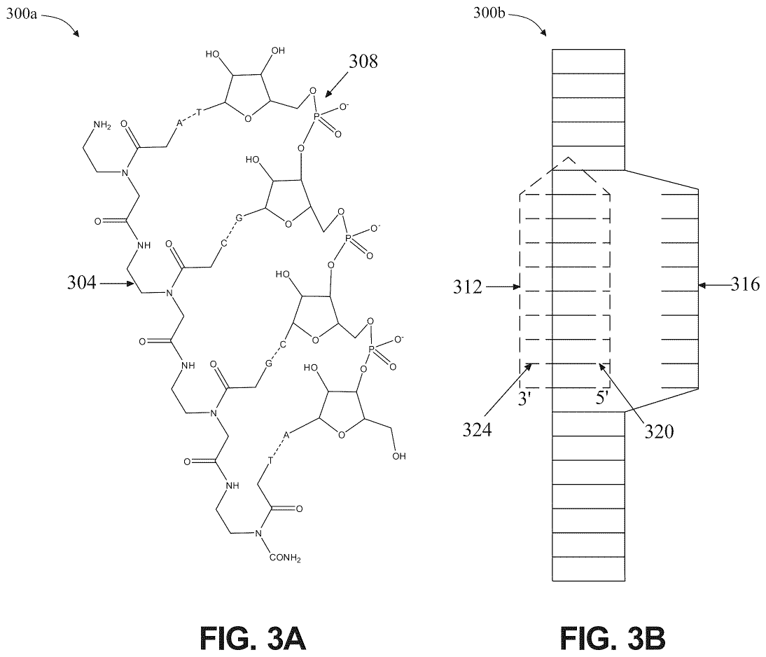

Now referring to FIGS. 3A-B, diagrammatic representations 300a-b of exemplary embodiments of a PNA are illustrated. In an embodiment, and without limitation, a PNA may include a class of nucleic acid targeting reagents that may demonstrate a strong affinity due to hybridization and/or a high specificity to their target cells when compared to a naturally occurring RNA and/or DNA molecule. Referring to FIG. 3A, a PNA may include a synthetic DNA analog in which a 2-N-aminoethylglycine unit 304 may act as a backbone. In an embodiment, and without limitation, 2-N-aminoethylglycine unit 304 may replace a phosphodiester bond in a DNA molecule. In another embodiment, 2-N-aminoethylglycine unit 304 may exhibit an increased stability in human blood serum and/or mammalian cellular extracts due to a lack of enzymatic cleavage. For example, and without limitation, phosphodiester bond 308 may be cleaved using a phosphodiesterase enzyme that catalyzes a hydrolysis of the phosphodiester bond 308, whereas 2-N-aminoethylglycine unit 304 may be unaffected by the phosphodiesterase enzyme, resulting in an enhanced stability. In an embodiment, and without limitation, PNA may be an attractive candidate for developing “cloning-free” nucleic acid therapies as a function of such increased stability and/or lack of enzymatic cleavage. Additionally, or alternatively, and now referring to FIG. 3B, PNA may include an antisense single-stranded PNA, which may be designed to bond to mRNA, wherein an antigene bis-PNA oligomer 312 may have the ability to bond to double-stranded DNA 316. As used in this disclosure, a “bis-PNA” is an oligomer that invades DNA 316 by forming a “triplex invasion complex”, wherein the triplex invasion complex includes two complementary homopyrimidine PNA strands connected to each other. For example, and without limitation, a first strand of PNA may target a homopurine DNA binding site as a function of a Watson-Crick base pairing binding 320, wherein a second strand of PNA may interact with the DNA strand using a Hoogsteen base pair binding 324, which may form a stable PNA₂-DNA triplex. As used in this disclosure, a “Watson-Crick base pair binding” is a chemical bond that a nucleobase may be secured by. For example, and without limitation, Watson-Crick base pair binding 320 may secure an adenine nucleobase to a thymine nucleobase. As a further nonlimiting example, Watson-Crick base pair binding 320 may secure a guanine nucleobase to a cytosine nucleobase. As used in this disclosure, a “Hoogsteen base pair binding” is a chemical bond that secures a nucleobase as a function of a hydrogen bond in a major groove. For example, and without limitation, Hoogsteen base pair binding 324 may secure one or more nucleobases as a function of the N7 position of a purine base and a C6 amino group position of the pyrimidine. As a further nonlimiting example, Hoogsteen base pair binding 324 may include one or more base pair bindings such that a triplex formation of oligonucleotides may form, such as but not limited to triplex-DNA, PNA-DNA triplex, and the like thereof. In an embodiment, and without limitation, PNA and/or antisense PNA may target one or more mRNAs to prevent protein expression, and/or anti-gene PNAs for transcriptional activation.

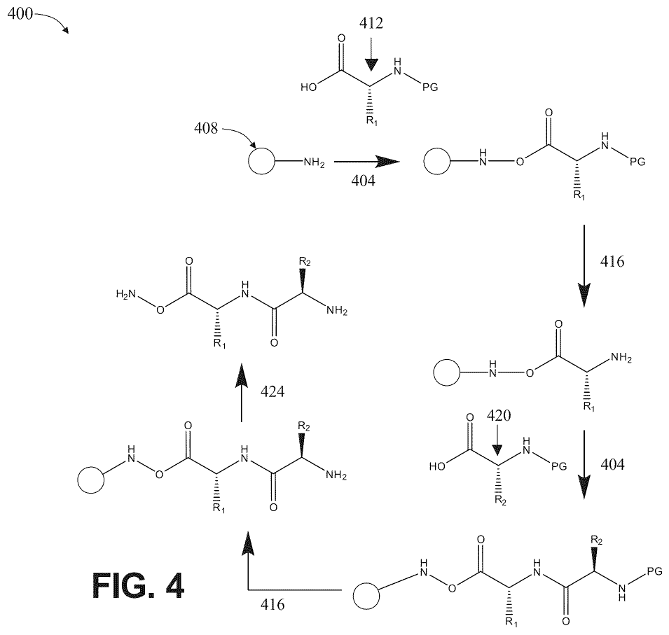

Now referring to FIG. 4, an exemplary embodiment 400 of a peptide synthesis is illustrated. In an embodiment, peptide synthesis may include a coupling process 404. As used in this disclosure, “coupling” is a process where two atoms of a molecule are joined together to form a chemical bond. In an embodiment, and without limitation, coupling process 404 may include a heterocoupling process. As used in this disclosure, a “heterocoupling process” is a process that combines two different chemical structures. For example, and without limitation, a heterocoupling process may include one or more processes such as a Heck reaction of an alkene and an alkyl halide to produce a substituted alkene. As a further nonlimiting example, a heterocoupling process may include a cross-coupling process, such as Cadiot-Chodkiewicz coupling, Castro-Stephens coupling, Corey-House synthesis, Kumada coupling, Sonogashira coupling, Negishi coupling, Stille cross-coupling, Suzuki reaction, Murahashi coupling, Hiyama coupling, Fukuyama coupling, Liebeskind-Srogl coupling, Ullmann-type reaction, Chan-Lam coupling, Buchwald-Hartwig reaction, palladium-catalyzed cross-coupling, and the like thereof. In an embodiment, and without limitation, coupling process 404 may include a homocoupling process. As used in this disclosure, a “homocoupling process” is a process that combines two identical chemical structures. For example, and without limitation, homocoupling process may include one or more processes such as a Glaser coupling process that couples and/or chemically bonds two acetylides, which may form a dialkyne. As a further nonlimiting example, a homocoupling process may include a process such as a Wurtz reaction, Pinacol coupling reaction, Ullmann reaction, and the like thereof.

Still referring to FIG. 4, coupling process 404 may include a resin 408. As used in this disclosure, a “resin” is solid support structure for peptide synthesis. Resin 408 may include any resin that comprises physical stability and/or permits the rapid filtration of liquids. In an embodiment and without limitation, resin 408 may include one or more gel-type support resins, surface-type support resins, and/or composite resins. In another embodiment, and without limitation, resin 408 may be able to withstand repeated use of trifluoroacetic acid (TFA). In another embodiment, resin 408 may include one or more resins as a function of a desired product such as, but not limited to a C-terminal carboxylic acid and/or an amide. In an embodiment, and without limitation, resin 408 may include a Wang resin. Additionally, or alternatively, coupling process 404 may include a first amino acid 412 to form a peptide. As used in this disclosure, a “peptide” is a short chain of between two and fifty amino acids linked by a peptide bond. In an embodiment and without limitation, first amino acid 412 may include an oligopeptide, dipeptide, tripeptide, tetrapeptide, and the like thereof. In another embodiment, and without limitation, first amino acid 412 may include an unbranched peptide chain. In another embodiment, and without limitation, first amino acid 412 may include a residue. As used in this disclosure, a “residue” is the portion of an amino acid, including its side chain, that remains once the amino acid has been incorporated into a peptide. Additionally, or alternatively first amino acid 412 may include one or more cyclic peptides including an N-terminal and/or an amine group and a C-terminal and/or a carboxyl group. In an embodiment, first amino acid 412 may include one or more plant peptides, bacterial and/or antibiotic peptides, fungal peptides, invertebrate peptides, amphibian and/or skin peptides, venom peptides, cancer and/or anticancer peptides, vaccine peptides, immune and/or inflammatory peptides, brain peptides, endocrine peptides, ingestive peptides, gastrointestinal peptides, cardiovascular peptides, renal peptides, respiratory peptides, opiate peptides, neurotrophic peptides, and blood-brain peptides, and/or the like thereof. In another embodiment, first amino acid 412 may include one or more post-translational modifications such as, but not limited to phosphorylated modifications, hydroxylated modifications, sulfonated modifications, palmitoylated modifications, disulfide modifications, and/or the like thereof. In an embodiment, and without limitation, first amino acid 412 may include a carboxylic acid component, an R1 component comprising a functional group containing a carbon and/or hydrogen atom, and/or an amine group bonded to a protecting group. As used in this disclosure, a “protecting group” is a functional group that stabilizes one or more reactive functional groups of a chemical and prevents undesirable side reactions thereof. For example, and without limitation, a protecting group may include a Boc or tBoc protecting group, an Fmoc protecting group, a carboxybenzyl (Cbz) protecting group, allyloxycarbonyl(Aloc) protecting group, and/or the like thereof. In an embodiment, and without limitation, first amino acid 412 may include a naturally occurring amino acid such as an L- or D-amino acid. In another embodiment, first amino acid 412 may include a synthetic amino acid, such as a glycine derivatized with a 2-aminoethyl group and/or a nucleoside/nucleobase, as described above.

Still referring to FIG. 4, a peptide synthesis may include a deprotection process 416. As used in this disclosure a “deprotection process” is a process that removes a protecting group from a reagent. For example, and without limitation, a deprotection process may deprotect an amino group to generate a free amino acid. In an embodiment, and without limitation, deprotection process 416 may include removing a protecting group using an acid, such as but not limited to trifluoroacetic acid, to form a positively charged amino group, wherein the positively charged amino group is then neutralized/deprotonated using a base. In another embodiment, and without limitation, deprotection process 416 may include removing the protecting group using a base, such as but not limited to piperidine. For example, and without limitation, removing a protecting group using a base may result in a neutral amine group, such that no neutralization/deprotonation is required. In an embodiment, and without limitation, A peptide synthesis may chemically bond and/or couple a second amino acid 420 as a function of coupling process 404, wherein the second amino acid 420 may include any of first amino acid 412 as described above. Second amino acid 420 may include a carboxylic acid component, an R2 component including a functional group containing a carbon and/or hydrogen atom, wherein an R2 component may include any of the R1 component structure and/or elements, and/or an amine group bonded to a protecting group, wherein protecting group is described above. In an embodiment, and without limitation, second amino acid 420 may be coupled to first amino acid 412, wherein the carboxylic acid component of second amino acid 420 may be coupled to the amine group of the first amino acid 412. In an embodiment and without limitation, second amino acid 420 may include a protecting group similar to first amino acid 412 or a protecting group distinct from the first amino acid 412. Coupling process 404 joins first amino acid 412 to second amino acid 420 to form a peptide chain and/or oligomer chain. In some cases, first amino acid 412 and/or second amino acid 420 may include a peptide bond. In some cases, first amino acid 412 and/or second amino acid 420 may include a peptide.

Still referring to FIG. 4, a peptide synthesis may deprotect a coupled product of first amino acid 412 and second amino acid 420 as a function of deprotecting process 416, consistent with details described above. In an embodiment, and without limitation, a peptide synthesis may be performed iteratively to produce a peptide chain and/or oligomer chain that is any number of peptides long. For example, and without limitation, a peptide synthesis may perform iterative processes to result in a peptide chain that is 23 peptides in length. Additionally, or alternatively, peptide synthesis may include a cleavage process 424. As used in this disclosure, a “cleavage process” is a process that removes resin from a synthesized peptide chain and/or oligomer chain. For example, and without limitation, cleavage process 424 may include using anhydrous hydrogen fluoride to cleave resin 408 from a peptide chain and/or oligomer chain. As a further nonlimiting example, cleavage process 424 may include using trifluoroacetic acid to cleave resin 408 from the peptide chain and/or oligomer chain.

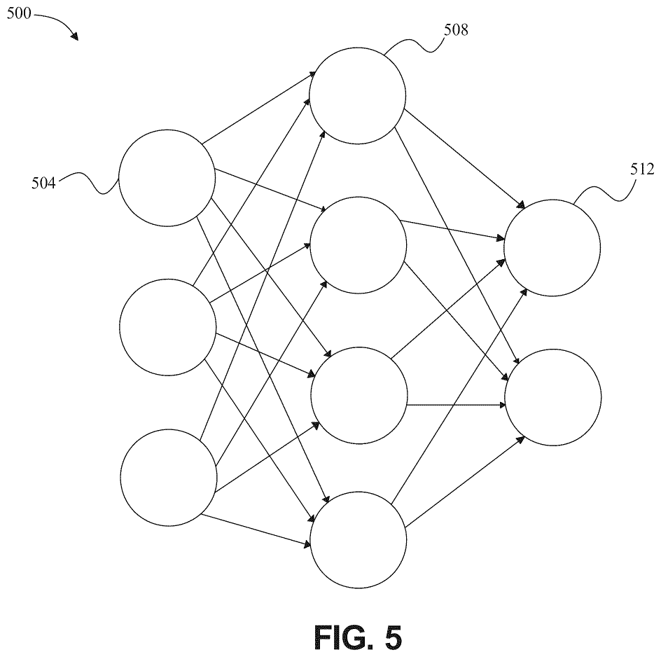

Referring now to FIG. 5, an exemplary embodiment of neural network 500 is illustrated. A neural network 500 also known as an artificial neural network, is a network of “nodes”, or data structures having one or more inputs, one or more outputs, and a function determining outputs based on inputs. Such nodes may be organized in a network, such as without limitation a convolutional neural network, including an input layer of nodes 504, one or more intermediate layers 508, and an output layer of nodes 512. Connections between nodes may be created via the process of “training” the network, in which elements from a training dataset are applied to the input nodes 504, a suitable training algorithm (such as Levenberg-Marquardt, conjugate gradient, simulated annealing, or other algorithms) is then used to adjust the connections and weights between nodes in adjacent layers 508 of the neural network to produce the desired values at the output nodes 512. This process is sometimes referred to as deep learning. Connections may run solely from input nodes toward output nodes in a “feed-forward” network or may feed outputs of one layer back to inputs of the same or a different layer in a “recurrent network.”

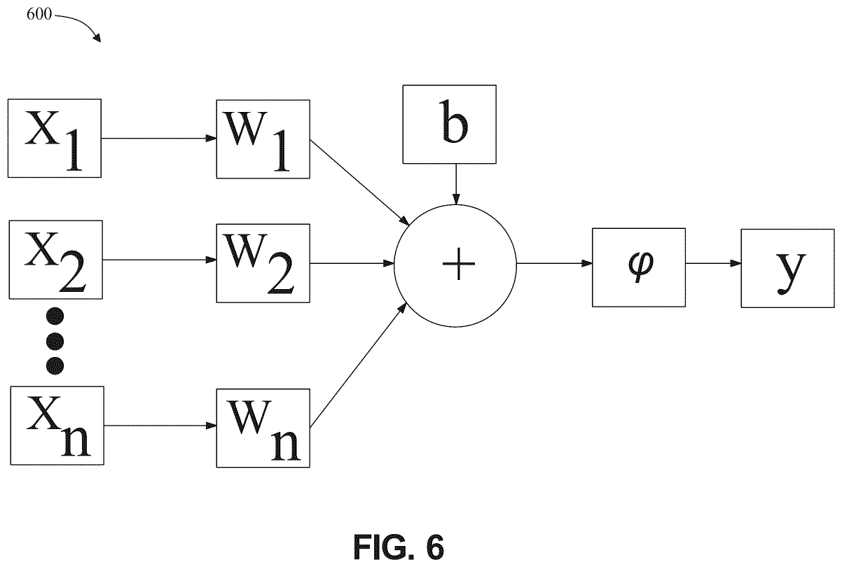

Referring now to FIG. 6, an exemplary embodiment 600 of a node of a neural network is illustrated. A node may include, without limitation, a plurality of inputs xi that may receive numerical values from inputs to a neural network containing the node and/or from other nodes. Node may perform a weighted sum of inputs using weights w; that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory”, indicating that it has strong influence on the one or more outputs y, for instance by the corresponding weight having a large numerical value, and/or an “inhibitory”, indicating it has a weak effect influence on the one or more inputs y, for instance by the corresponding weight having a small numerical value. The values of weights wᵢ may be determined by training a neural network using training data, which may be performed using any suitable process as described above.

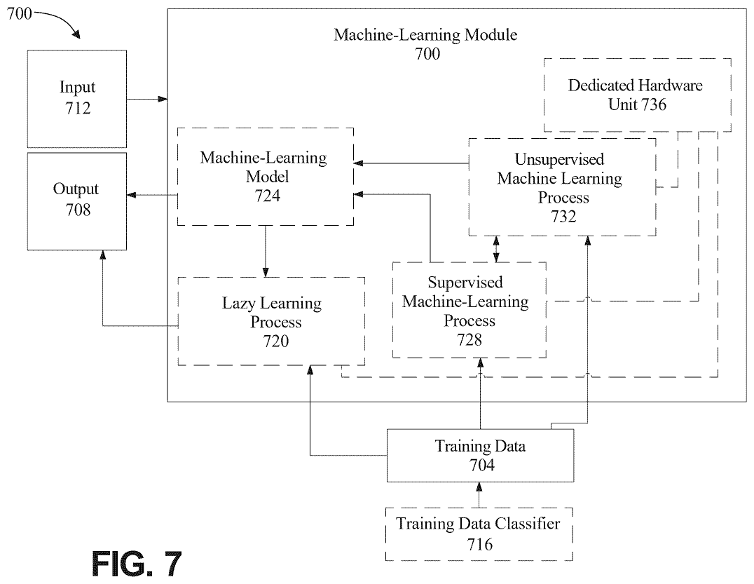

Referring now to FIG. 7, an exemplary embodiment of a machine-learning module 700 that may perform one or more machine-learning processes as described in this disclosure is illustrated. A machine-learning module may perform determinations, classification, and/or analysis steps, methods, processes, or the like as described in this disclosure using machine-learning processes. A “machine-learning process”, as used in this disclosure, is a process that automatically uses training data 704 to generate an algorithm that will be performed by a computing device/module to produce outputs 708 given data provided as inputs 712. This is in contrast to a non-machine-learning software program where the commands to be executed are determined in advance by a user and written in a programming language.

Still referring to FIG. 7, “training data”, as used herein, is data containing correlations that a machine-learning process may use to model relationships between two or more categories of data elements. For instance, and without limitation, training data 704 may include a plurality of data entries, each entry representing a set of data elements that were recorded, received, and/or generated together; data elements may be correlated by shared existence in a given data entry, by proximity in a given data entry, or the like. Multiple data entries in training data 704 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 704 according to various correlations; correlations may indicate causative and/or predictive links between categories of data elements, which may be modeled as relationships such as mathematical relationships by machine-learning processes as described in further detail below. Training data 704 may be formatted and/or organized by categories of data elements, for instance by associating data elements with one or more descriptors corresponding to categories of data elements. As a nonlimiting example, training data 704 may include data entered in standardized forms by persons or processes, such that entry of a given data element in a given field in a form may be mapped to one or more descriptors of categories. Elements in training data 704 may be linked to descriptors of categories by tags, tokens, or other data elements; for instance, and without limitation, training data 704 may be provided in fixed-length formats, formats linking positions of data to categories such as comma-separated value (CSV) formats, and/or self-describing formats such as extensible markup language (XML), JavaScript Object Notation (JSON), or the like, enabling processes or devices to detect categories of data.

Alternatively, or additionally, and continuing to refer to FIG. 7, training data 704 may include one or more elements that are not categorized; that is, training data 704 may not be formatted or contain descriptors for some elements of data. Machine-learning algorithms and/or other processes may sort training data 704 according to one or more categorizations using, for instance, natural language processing algorithms, tokenization, detection of correlated values in raw data and the like; categories may be generated using correlation and/or other processing algorithms. As a nonlimiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatically may enable the same training data 704 to be made applicable for two or more distinct machine-learning algorithms as described in further detail below. Training data 704 used by machine-learning module 700 may correlate any input data as described in this disclosure to any output data as described in this disclosure. As a nonlimiting illustrative example, inputs may include prospective gene targets, wherein outputs may include a proposed therapeutic oligomer sequence.

Further referring to FIG. 7, training data may be filtered, sorted, and/or selected using one or more supervised and/or unsupervised machine-learning processes and/or models as described in further detail below; such models may include without limitation a training data classifier 716. A “classifier”, as used in this disclosure, is a machine-learning model that sorts inputs into categories or bins of data, outputting the categories or bins of data and/or labels associated therewith. A classifier may include a mathematical model, a neural net, or a program generated by a machine-learning algorithm known as a “classification algorithm”, as described in further detail below. A classifier may be configured to output at least a datum that labels or otherwise identifies a set of data that are clustered together, found to be close under a distance metric as described below, or the like. Machine-learning module 700 may generate a classifier using a classification algorithm, whereby a computing device and/or any module and/or component operating thereon derives a classifier from training data 704. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 7, machine-learning module 700 may be configured to perform a lazy-learning process 720 and/or protocol, which may alternatively be referred to as a “lazy loading” or “call-when-needed” process and/or protocol. Lazy-learning process 720 may be a process whereby machine learning is conducted upon receipt of an input to be converted to an output, by combining the input and training set to derive an algorithm to be used to produce the output on demand. For instance, an initial set of simulations may be performed to cover an initial heuristic and/or “first guess” at an output and/or relationship. As a nonlimiting example, an initial heuristic may include a ranking of associations between inputs and elements of training data 704. Heuristic may include selecting some number of highest-ranking associations and/or training data 704 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 7, machine-learning processes as described in this disclosure may be used to generate machine-learning models 724. A “machine-learning model”, as used in this disclosure, is a mathematical and/or algorithmic representation of a relationship between inputs and outputs, as generated using any machine-learning process including without limitation any process as described above and stored in memory; an input is submitted to a machine-learning model 724 once created, which generates an output based on the relationship that was derived. For instance, and without limitation, a linear regression model, generated using a linear regression algorithm, may compute a linear combination of input data using coefficients derived during machine-learning processes to calculate an output datum. As a further nonlimiting example, a machine-learning model 724 may be generated by creating an artificial neural network, such as a convolutional neural network, that include an input layer of nodes, one or more intermediate layers, and an output layer of nodes. Connections between nodes may be created via a process of “training” the network, in which elements from a training data 704 set are applied to input nodes, and a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 7, machine-learning algorithms may include at least a supervised machine-learning process 728. At least a supervised machine-learning process 728, as defined herein, includes algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs as described above in this disclosure, and a scoring function representing a desired form of relationship to be detected between inputs and outputs. A scoring function may, for instance, seek to maximize the probability that a given input and/or combination of inputs elements is associated with a given output to minimize the probability that a given input is not associated with a given output. A scoring function may be expressed as a risk function representing an “expected loss” of an algorithm relating inputs to outputs, where the loss is computed as an error function representing a degree to which a prediction generated by the relation is incorrect when compared to a given input-output pair provided in training data 704. Persons skilled in the art, upon reviewing the entirety of this disclosure, will be aware of various possible variations of at least a supervised machine-learning process 728 that may be used to determine relation between inputs and outputs. Supervised machine-learning processes may include classification algorithms as defined above.

Further referring to FIG. 7, machine-learning processes may include at least an unsupervised machine-learning process 732. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 7, machine-learning module 700 may be designed and configured to create a machine-learning model 724 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 7, machine-learning algorithms may include, without limitation, linear discriminant analysis. Machine-learning algorithms may include quadratic discriminate analysis. Machine-learning algorithms may include kernel ridge regression. Machine-learning algorithms may include support vector machines, including, without limitation, support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

With continued reference to FIG. 7, a machine-learning model and/or process may be deployed or instantiated by incorporation into a program, apparatus, system, and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit, to represent a number according to any suitable encoding system including twos complement or the like, or may be stored in any volatile and/or non-volatile memory. Similarly, mathematical operations and input 712 and/or output 708 of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation application-specific integrated circuits (ASICs), production and/or configuration of reconfigurable hardware elements, circuits, and/or modules such as without limitation field programmable gate arrays (FPGAs), production and/or configuration of non-reconfigurable and/or non-rewritable memory elements, circuits, and/or modules such as without limitation non-rewritable read-only memory (ROM), other memory technology described in this disclosure, and/or production and/or configuration of any computing device and/or component thereof as described in this disclosure. Such deployed and/or instantiated machine-learning model and/or algorithm may receive inputs 712 from any other process, module, and/or component described in this disclosure, and produce outputs 708 to any other process, module, and/or component described in this disclosure.

With continued reference to FIG. 7, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively, or additionally, retraining, deployment, and/or instantiation may be event-based, and may be triggered, without limitation, by user inputs indicating sub-optimal or otherwise problematic performance and/or by automated field testing and/or auditing processes, which may compare outputs 708 of machine-learning models and/or algorithms, and/or errors and/or error functions thereof, to any thresholds, convergence tests, or the like, and/or may compare outputs 708 of processes described herein to similar thresholds, convergence tests or the like. Event-based retraining, deployment, and/or instantiation may alternatively, or additionally, be triggered by receipt and/or generation of one or more new training examples; a number of new training examples may be compared to a preconfigured threshold, where exceeding the preconfigured threshold may trigger retraining, deployment, and/or instantiation.

With continued reference to FIG. 7, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data 704 may include, without limitation, training examples including inputs 712 and correlated outputs 708 used, received, and/or generated from any version of any system, module, machine-learning model or algorithm, apparatus, and/or method described in this disclosure. Such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs 708 for training processes as described above. Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

With continued reference to FIG. 7, one or more processes or algorithms described above may be performed by at least a dedicated hardware unit 736. For the purposes of this disclosure, a “dedicated hardware unit” is a hardware component, circuit, or the like, aside from a principal control circuit and/or computing device 104 performing method steps as described in this disclosure, which is specifically designated or selected to perform one or more specific tasks and/or processes described in reference to this figure. Such specific tasks and/or processes may include without limitation preprocessing and/or sanitization of training data and/or training a machine-learning algorithm and/or model. Dedicated hardware unit 736 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously, in parallel, and/or the like. Such dedicated hardware units 736 may include, without limitation, graphical processing units (GPUs), dedicated signal processing modules, field programmable gate arrays (FPGA), other reconfigurable hardware that has been configured to instantiate parallel processing units for one or more specific tasks, or the like. Computing device 104, system 100, or machine-learning module 700 may be configured to instruct one or more dedicated hardware units 736 to perform one or more operations described herein, such as evaluation of model and/or algorithm outputs, one-time or iterative updates to parameters, coefficients, weights, and/or biases, vector and/or matrix operations, and/or any other operations described in this disclosure.

Now referring to FIG. 8, an exemplary embodiment 800 of a modular approach is illustrated. As used in this disclosure, a “modular approach” is a method and/or process to design proposed therapeutic oligomer sequence 108. In an embodiment, and without limitation, modular approach may include determining a genomic binding domain 804. As used in this disclosure, a “genomic binding domain” is a genomic sequence of interest and/or a genomic sequence to be regulated. For example, and without limitation, genomic binding domain 804 may include a DNA binding domain and/or an RNA binding domain. In an embodiment, and without limitation, determining genomic binding domain 804 may include identifying a target. As used in this disclosure, a “target” is an intended pathogen, viral agent, and/or bacterium to be innervated. For example, and without limitation, a target may include a virus, a bacterium, a microbe, and the like thereof. In an embodiment, and without limitation, determining genomic binding domain 804 may include determine a unique identifier. As used in this disclosure, a “unique identifier” is an identifier and/or signature associated to the target. In an embodiment, and without limitation, a unique identifier may denote that a target is unique to a specific pathogen, viral agent, and/or bacteria. For example, and without limitation, a unique identifier may include a unique genomic sequence and/or unique nucleobase pair sequence. Modular approach may include determining a localization sequence 808. As used in this disclosure, a “localization sequence” is a target location to be innervated in a target. In an embodiment, and without limitation, localization sequence 808 may include a nuclear localization sequence. For example, and without limitation, nuclear localization sequence may denote that genomic binding domain 804 is located within a nucleus of a cell. As a further nonlimiting example, nuclear localization sequence may denote that genomic binding domain 804 is used for transcriptional inhibition. As a further nonlimiting example, nuclear localization sequence may denote that genomic binding domain 804 may be intended for activation.

Still referring to FIG. 8, modular approach may include identifying an activation domain 812. As used in this disclosure, an “activation domain” or “transcriptional activation domain” is a location that may be used to activate transcription from a promoter. In one or more embodiments, an activation domain may be a defined genomic DNA region associated with a promoter that is selected as a functional site at which binding of a synthetic regulatory construct increases transcriptional output from the promoter. In one or more embodiments, activation domain may include a nucleotide sequence located within, adjacent to, or upstream of a transcription start site, and may overlap regulatory features such as promoter elements, enhancer-like motifs, transcription factor binding sites, or chromatin-accessible regions. In one or more embodiments, activation domain may be identified through computational analysis, empirical data, or a combination thereof, as a locus at which targeted binding results in increased transcription by facilitating recruitment of transcriptional machinery, promoting chromatin opening, or displacing repressive regulatory factors. In one or more embodiments, activation domain may be used as a modular targeting site to which a sequence-specific binding element is directed, wherein the binding element may be operably linked to a transcriptional activation function. In one or more embodiments, binding of a construct at activation domain may increase a frequency or efficiency of transcription initiation by enhancing RNA polymerase recruitment, stabilizing pre-initiation complexes, or modifying local chromatin structure. In an embodiment, and without limitation, activation domain 812 may include acidic domains. As used in this disclosure, an “acidic domain” is a domain including a large quantity of D and E amino acids. In an embodiment, and without limitation, an acidic domain may include well-known activators such as Gal4, Gcn4, VP16, p53, p53, and/or the like thereof. In another embodiment, and without limitation, acidic domain may include one or more amino acid sequences including E TFSD LWKL (SEQ ID NO: 12), D DIEQ WFTE (SEQ ID NO: 13), S DIMD FVLK (SEQ ID NO: 14), D LLDF SMMF (SEQ ID NO: 15), E TLDF SLVT (SEQ ID NO: 16), R KILN DLSS (SEQ ID NO: 17), E AILA ELKK (SEQ ID NO: 18), D DVVQ YLNS (SEQ ID NO: 19), D DVYN YLFD (SEQ ID NO: 20), D LFDY DFLV (SEQ ID NO: 21), D FFDY DLLF (SEQ ID NO: 22), E DLYS ILWS (SEQ ID NO: 23), T DLYH TLWN (SEQ ID NO: 24), and/or the like thereof. In another embodiment, and without limitation, activation domain 812 may include glutamine-rich domains, proline-rich peptide domains, isoleucine-rich peptide domains, and the like thereof. Additionally, or alternatively, modular approach may include identifying a nanoparticle-binding domain 816. As used in this disclosure, a “nanoparticle-binding domain” is a location that may be used to bind to a nanoparticle. For example, and without limitation, nanoparticle-binding domain 816 may denote that an oligomer and/or genomic sequence domain may be capable of binding to a nanoparticle. In an embodiment, and without limitation, nanoparticle-binding domain 816 may denote that the size of a nanoparticle is less than 2 nm. In another embodiment, and without limitation, nanoparticle-binding domain 816 may denote that a nanoparticle may be fluorescent. As a further nonlimiting example, nanoparticle-binding domain 816 may denote that a nanoparticle is FDA approved. In an embodiment, and without limitation, nanoparticle binding domain may denote that a nanoparticle is comprised of gold, zinc, iron oxide, and/or the like thereof.

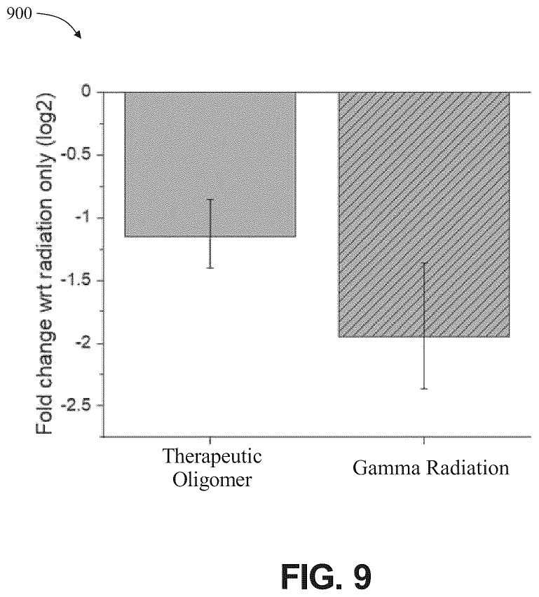

Now referring to FIG. 9, an exemplary embodiment 900 of a regulation modification is illustrated. In an embodiment, and without limitation, therapeutic oligomer 156 may downregulate one or more inflammatory genes such as IL-1α when compared to gamma-radiation stimulated inflammatory genes in donor-derived human PBMCs. In another embodiment, and without limitation, regulation modification may downregulate one or more proinflammatory cytokines. For example, and without limitation, regulation modification may regulate G-CSF and/or Colony-stimulating factor 3 (CSF-3), Stromal cell-derived factor 1 (SDF-1), Epicatchetin (EC) as a small-molecule reactive oxygen species (ROS) inhibitor comparison, Erythropoietin (EPO), Stem cell factor (SCF), TNF-α; Thrombopoietin (THPO), Fms related tyrosine kinase 3 ligand (FLT3LG), IL-3, IL-1α, THPO-isoform, IL-6, IL-1β, and/or CSF-2. In another embodiment, and without limitation, therapeutic oligomer 156 may downregulate one or more inflammatory genes such as IL-1β when compared to gamma-radiation stimulated inflammatory genes in donor-derived human peripheral blood mononuclear cells (PBMCs). In another embodiment, and without regulation, therapeutic oligomer 156 may downregulate one or more inflammatory genes such as IL-6 when compared to gamma-radiation stimulated inflammatory genes in donor-derived human PBMCs. A 3-fold downregulation of IL-6 gene, compared to untreated PBMCs, may be observed. Additionally, or alternatively, regulation modification may downregulate more than 14 additional proinflammatory cytokines associated to neurodegeneration.

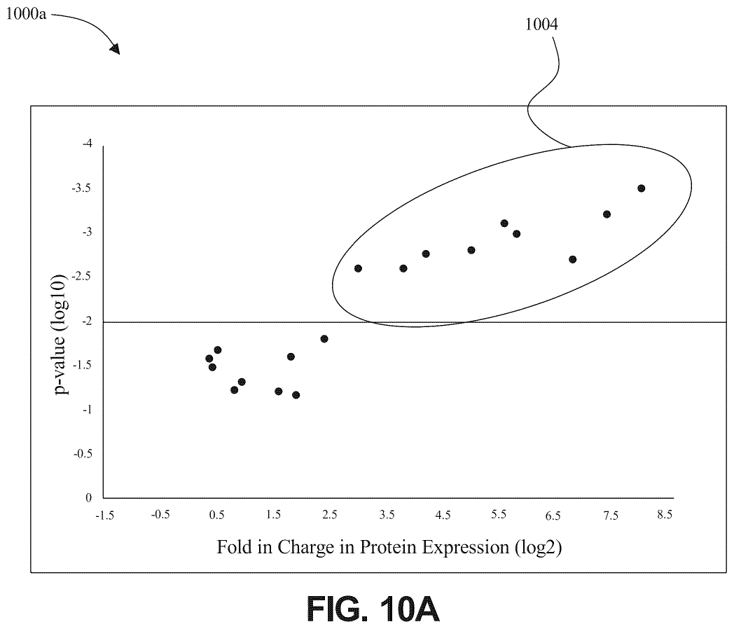

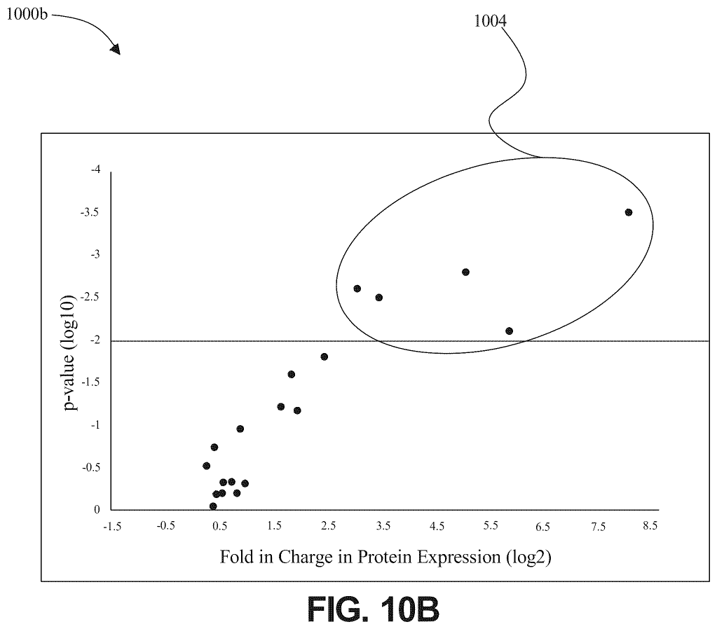

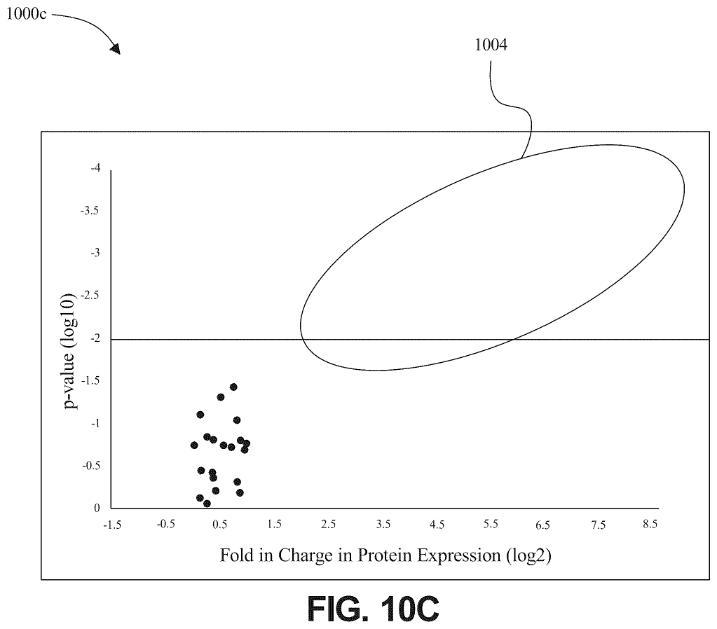

Now referring to FIGS. 10A-C, exemplary embodiments 1000a-c of a therapeutic effect is illustrated. In an embodiment, and without limitation, therapeutic effect may denote a reduced neuroinflammation in cytokine-stimulated primary human astrocytes. For example, and without limitation, and referring to FIG. 10A, untreated primary human astrocytes may result in a strong inflammation as seen in a cytokine cocktail. A cytokine cocktail may include but is not limited to IL-1 IL-1α, TNF-α, and/or complement component C1q, wherein stimulated cells may release proinflammatory cells that are measured as a function of a p-value and/or enrichment and fold-change with respect to untreated human astrocytes. In an embodiment, untreated primary human astrocytes may denote a high inflammation denoted by an inflammatory grouping 1004. As used in this disclosure, an “inflammatory grouping” is a collection of data points that depict one or more frequencies of proinflammatory cell stimulation that exist below a p-value of −2. For example, inflammatory grouping in untreated cells may include a large frequency of proinflammatory cell stimulation. Now referring to FIG. 10B, primary human astrocytes treated with small molecule NF-kβ inhibitor may reduce neuroinflammation as a function of a reduced inflammatory grouping 1004. Additionally, or alternatively, and now referring to FIG. 10C, therapeutic oligomer 156 may eliminate neuroinflammation as a function of an eliminated inflammatory grouping 1004.

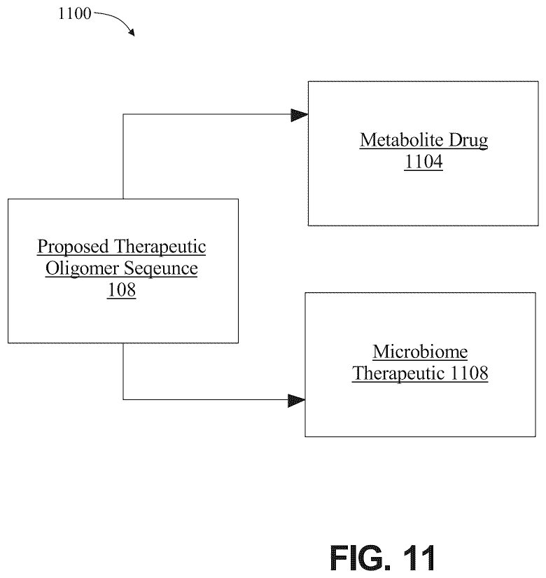

Now referring to FIG. 11, an exemplary embodiment 1100 of proposed therapeutic oligomer sequence 108 is illustrated. Proposed therapeutic oligomer sequence 108 may include a sequence to stimulate a metabolite drug 1104. As used in this disclosure, a “metabolite drug” is a bacterial metabolite that directs a microbiome towards a desired state. In an embodiment, and without limitation, metabolite drug 1104 may include bacterial metabolites that include growth-promoting and/or growth-inhibiting factors. For example, and without limitation, metabolite drug 1104 may include a bacterial metabolite such as granulocyte-macrophage colony-stimulating factor (GM-CSF), granulocyte colony-stimulating factor (G-CSF), and the like thereof. Proposed therapeutic oligomer sequence 108 may include a sequence that encodes a microbiome therapeutic 1108. As used in this disclosure, a “microbiome therapeutic” is a therapeutic that directs a microbiome towards a desired state. In an embodiment, and without limitation, microbiome therapeutic 1108 may include therapeutics such as antisense nanoligomers that may target a peptidase domain-containing ABC transporter gene.

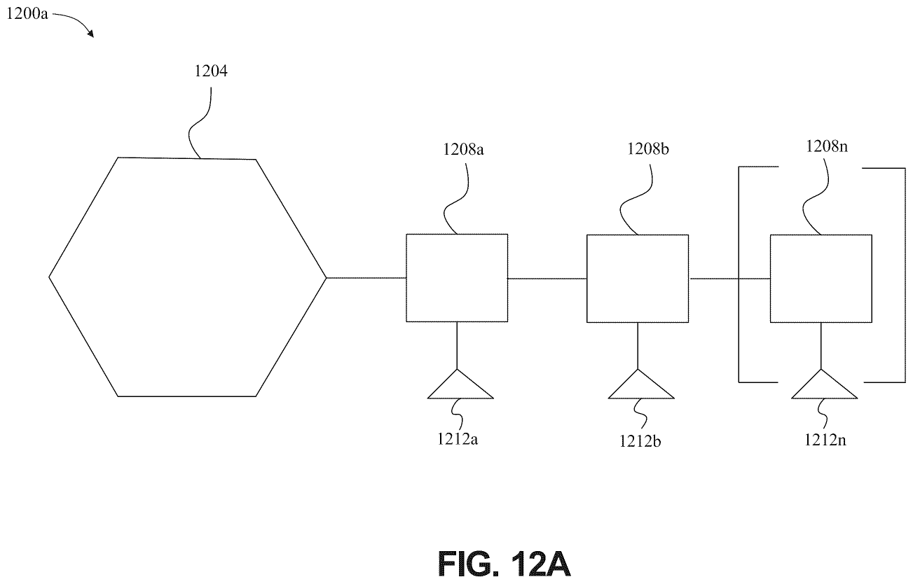

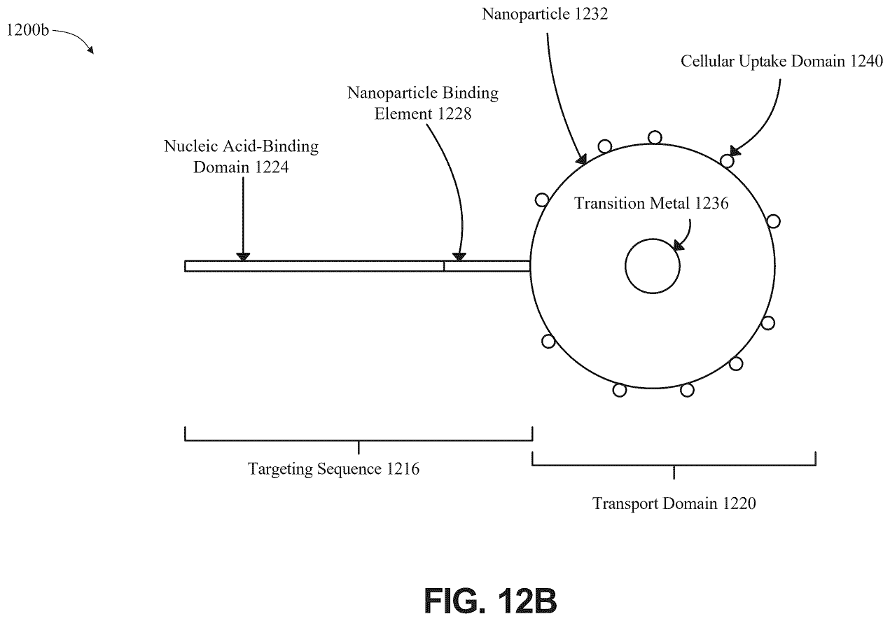

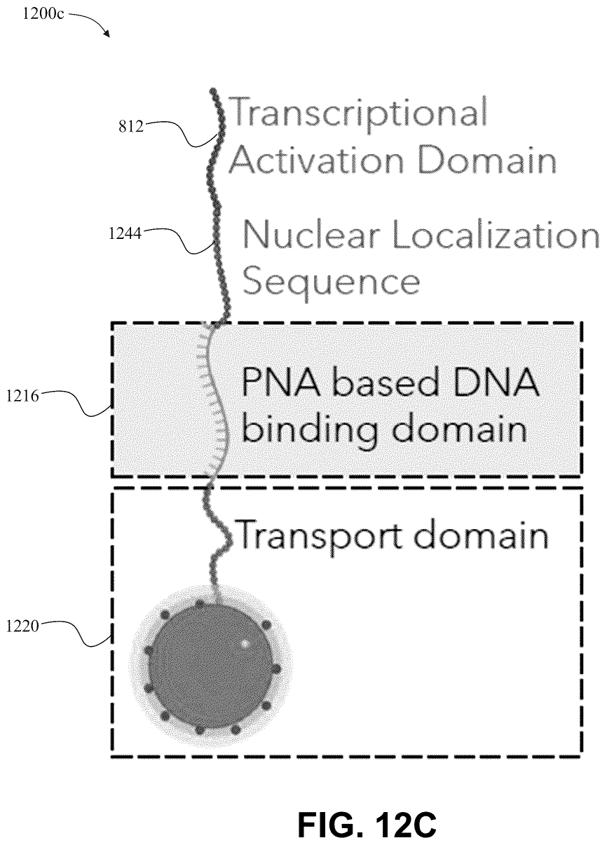

Now referring to FIG. 12A, an exemplary embodiment 1200a of a therapeutic oligomer 156 is illustrated. In an embodiment, and without limitation, therapeutic oligomer 156 may include a support structure 1204. As used in this disclosure, a “support structure” is a physically stable structure with binding affinity to a molecule and/or polymer. In an embodiment, and without limitation, support structure 1204 may include a nanoparticle, consistent with details described above in this disclosure.

Still referring to FIG. 12A, therapeutic oligomer 156 may include a first backbone element 1208a. As used in this disclosure, a “backbone element” is a chemical and/or molecule that attaches, binds, or otherwise connects to another chemical and/or molecule through a bond. In an embodiment, and without limitation, backbone element 1208a may include N-(2-aminoethyl)-glycine, consistent with details described above. In another embodiment, and without limitation, backbone element 1208a may include deoxyribose. In another embodiment, and without limitation, backbone element 1208a may include ribose. In another embodiment, and without limitation, backbone element 1208a may include one or more neutral and/or charged phosphate groups. Additionally, or alternatively, therapeutic oligomer 156 may include a second backbone element 1208b. Second backbone element 1208b may include any chemical and/or molecule applicable to first backbone element 1208a. In an embodiment, and without limitation, therapeutic oligomer 156 may include a plurality of backbone elements 1208n. In an embodiment, and without limitation, plurality of backbone elements 1208n may be coupled via one or more covalent bonds, as described above in reference to FIGS. 1-11.

Still referring to FIG. 12A, therapeutic oligomer 156 may include a first nucleobase 1212a. As used in this disclosure, a “nucleobase” is a chemical and/or molecule including a nitrogenous base. In an embodiment, and without limitation, first nucleobase 1212a may include one or more chemicals and/or molecules such as but not limited to adenine (A), cytosine (C), guanine (G), thymine (T), uracil (U), and the like thereof. In another embodiment, and without limitation, first nucleobase 1212a may include one or more primary and/or canonical nucleobases. In another embodiment, and without limitation, first nucleobase 1212a may include a purine base and/or a pyrimidine base. In another embodiment, and without limitation, first nucleobase 1212a may include one or more chemicals and/or molecules such as but not limited to xanthine, hypoxanthine, 2,6-diaminopurine, 6,8-diaminopurine, and/or the like thereof. In another embodiment, and without limitation, first nucleobase 1212a may include one or more chemicals and/or molecules such as but not limited 7-methylguanine, inosine, 7-methylguanosine, 5,6-dihydrouracil, 5-methylcytosine, 5-hydroxymethylcytosine, dihydrouridine, 5-methylcytidine, and the like thereof, and the like thereof. In another embodiment, and without limitation, first nucleobase 1212a may include one or more chemicals and/or molecules such as but not limited to aminoallyl nucleotides, isoguanine, isocytosine, 2-amino-6 (2-thienyl) purine, pyrrole-2-carbaldehyde, and the like thereof. Additionally, or alternatively, therapeutic oligomer 156 may include a second nucleobase 1212b. Second nucleobase 1212b may include any chemical and/or molecule applicable to first nucleobase 1212a. In an embodiment, and without limitation, therapeutic oligomer 156 may include a plurality of nucleobases 1212n.

Now referring to FIG. 12B, an exemplary embodiment 1200b of a nanoligomer is illustrated, consistent with details described above in this disclosure. A nanoligomer may include a targeting sequence 1216. A nanoligomer may include a transport domain 1220 that functions as a delivery vehicle. In some cases, targeting sequence 1216 may include a nucleic acid-binding domain 1224 that hybridizes, pairs, or otherwise interacts with gene target 116 to achieve an intended therapeutic effect. In some cases, targeting sequence 1216 may include a nanoparticle binding element 1228 that anchors to transport domain 1220. In some cases, nanoparticle binding element 1228 may include a linker. Transport domain 1220 may include a nanoparticle 1232 functionalized with one or more cellular uptake domains 1240. In some cases, nanoparticle 1232 may include a transition metal 1236.

Now referring to FIG. 12C, an exemplary embodiment 1200c of a nanoligomer is illustrated, consistent with details described above in this disclosure. A nanoligomer may include a transport domain 1220, as described above. A nanoligomer may include a targeting sequence 1216, such as a PNA-based DNA binding domain. Additionally, a nanoligomer may include an NLS 1244, consistent with details described above in this disclosure. Additionally, a nanoligomer may include a (transcriptional) activation domain 812, consistent with details described above.

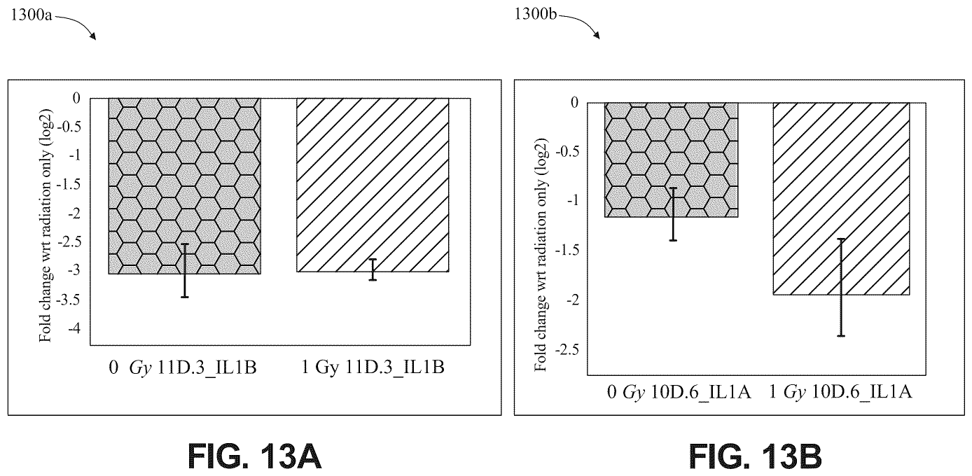

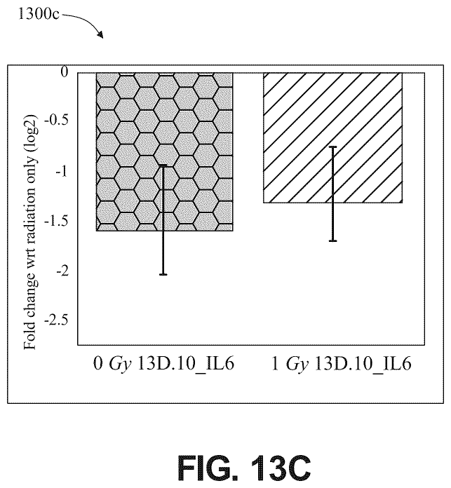

Now referring to FIGS. 13A-C, exemplary embodiments 1300a-c of downregulation using therapeutic nanoligomers are illustrated. In an embodiment, therapeutic nanoligomers may downregulate key proinflammatory genes implicated in neurodegeneration. Now referring to FIG. 13A, therapeutic nanoligomer 11D.3_IL1B may downregulate of IL-1B 8-fold in both unstimulated (0 Gray or Gy) and gamma-radiation stimulated (1 Gray or Gy) donor-derived human peripheral blood mononuclear cells (PBMCs) in comparison to untreated PBMCs (control). Now referring to FIG. 13B, therapeutic nanoligomer 10D.6IL1A may downregulate IL-1α more than 2-3-fold in comparison to untreated PBMCs. Now referring to FIG. 13C, therapeutic nanoligomer 13D.10_IL6 may downregulate IL-6 gene more than 3-fold in comparison to untreated PBMCs. In an embodiment, and without limitation, more than 14-key proinflammatory cytokines implicated in neurodegeneration may be downregulated through therapeutic nanoligomers.

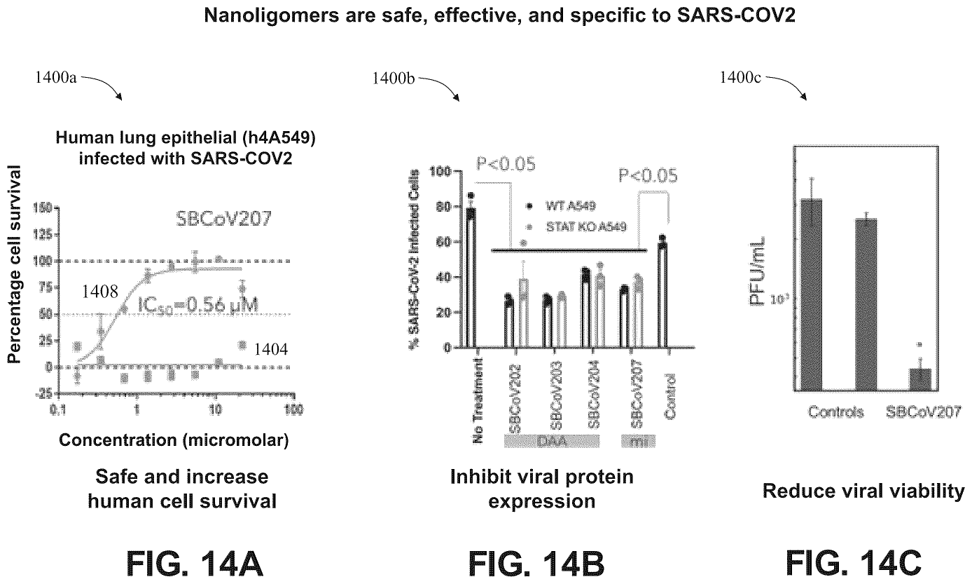

Now referring to FIGS. 14A-C, exemplary embodiments 1400a-c of an antiviral therapeutic oligomer is illustrated. Now referring to FIG. 14A, a cytotoxicity and cell survival graph is represented. Graph may include a cytotoxicity element 1404 denoting the safety and/or cytotoxicity of a SARS-COV-2 antiviral, wherein the SARS-COV-2 antiviral may be host-directed, targeting microRNA mi2392. In an embodiment, cytotoxicity element 1404 may be represented as a curve on the graph. In another embodiment, graph may include a cell survival element 1408 denoting the cell survival of human lung epithelial cells (hA4549) infected with a SARS-COV-2 viral agent. In an embodiment, and without limitation, cytotoxicity element 1404 and cell survival element 1408 may demonstrate the safety and/or efficacy in an in vitro infection model wherein limited and/or no cytotoxicity occurs. A restoration of survival may be observed in the presence of 3-5 μM SARS-COV-2 antiviral. Now referring to FIG. 14B, a viral clearance in an in vitro assay is represented. In an embodiment, and without limitation, viral clearance in an in vitro assay may show elimination of viral load using fluorescence tagging of viral protein and using three direct-acting and one host-directed SARS-COV2 antivirals. Significant reduction of viral protein may demonstrate the ability of a nanoligomer to clear out an infection, even in STAT-3 knockout cells. Now referring to FIG. 14C, an efficacy of clearing infection of a SARS-CoV-2 viral agent is represented. In an embodiment, and without limitation, a reduction in the plaque-forming unit (PFU) of the viral load may demonstrate the efficacy of clearing a SARS-CoV2 infection using a therapeutic nanoligomer as antiviral and anti-infectives.

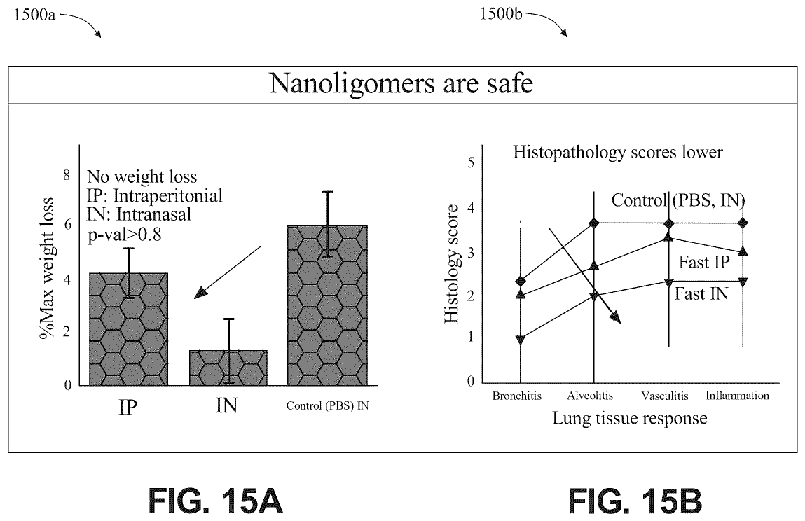

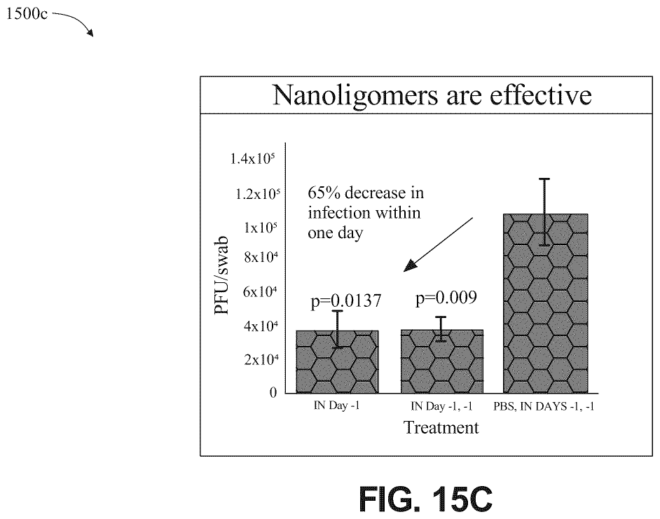

Now referring to FIGS. 15A-C, exemplary embodiments 1500a-c of an in vivo efficacy assessment of a therapeutic oligomer 156 for treating SARS-COV-2 are illustrated. In an embodiment and without limitation, an in vivo efficacy assessment of therapeutic oligomer 156 may be determined using an in vivo assay in Syrian Hamsters, wherein the safety and efficacy assessment of a designed host-directed nanoligomer may be evaluated using weight changes in key organs, histopathology scores, and/or plaque assays retrieved using a swab. Now referring to FIG. 15A, two different routes of administration, intranasal (IN) and intraperitoneal (IP), may be analyzed, wherein significant/statistical increases may be observed in weight loss, indicating the safety of an administered therapeutic oligomer 156. Now referring to FIG. 15B, histopathology scores for both routes of administration may be lower than a control group, wherein a lower score may indicate a healthier organ with a high tolerability and/or safety profile of an administrated therapeutic oligomer 156. A safety profile may represent a lack of any toxicity of inflammation, Now referring to FIG. 15C, the results of a plaque assay are illustrated, showing an efficacy measured in a reduced PFU of the viral load. Such reduction indicates the clearing of a SARS-COV-2 infection using therapeutic oligomer 156 as an effective antiviral.

Now referring to FIG. 16, an exemplary embodiment 1600 of a therapeutic effect of a therapeutic nanoligomer is illustrated. In an embodiment, and without limitation, a therapeutic nanoligomer may have a therapeutic effect of a significant reduction of key proinflammatory cytokines. For example, and without limitation, molecular targets may comprise G-CSF and/or Colony-stimulating factor 3 (CSF-3), Stromal cell-derived factor 1 (SDF-1), Epicatchetin (EC) as small molecule ROS inhibitor comparison, Metformin as small molecule non-sulfonylurea comparison, Aspirin as a small molecule comparison, Erythropoietin (EPO), Stem cell factor (SCF), TNF-α, Thrombopoietin (THPO), fms related tyrosine kinase 3 ligand (FLT3LG), IL-3, IL-1α, THPO-isoform, IL-6, IL-1β, and/or CSF-2. In an embodiment, and without limitation, a therapeutic nanoligomer may downregulate one or more molecular targets by downregulating one or more targeted cytokines/genes. In another embodiment, and without limitation, therapeutic nanoligomer may reduce inflammation by downregulating cytokine expression of key inflammatory cytokines, such as during neurodegeneration.

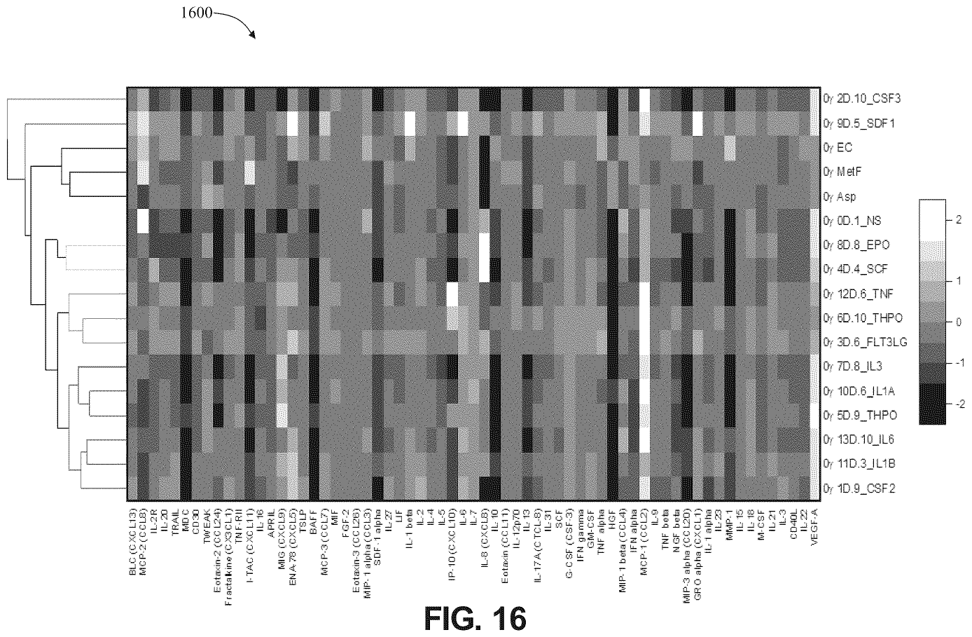

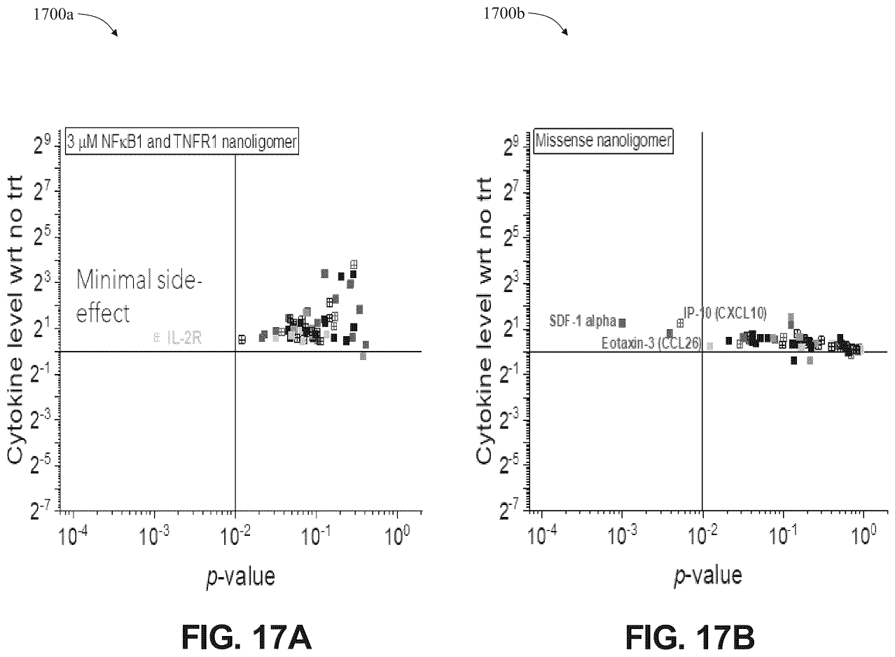

Now referring to FIGS. 17A-B, exemplary embodiments 1700a-b of a toxicity of therapeutic oligomer 156 are illustrated. Referring to FIG. 17A, therapeutic oligomer 156 for neurotherapeutic NF-kβ inhibitor and/or TNF-receptor 1 inhibitor may be applied to human astrocytes, wherein no immunotoxicity may be detected. Referring to FIG. 17B, therapeutic oligomer 156 with a missense nanoligomer may not show any immunotoxicity. In an embodiment, and without limitation, cytokine release syndrome may not be directly related to immunogenicity, wherein a clinical presentation of cytokine release syndrome may overlap with anaphylaxis and other immunologically related adverse reactions. In another embodiment, and without limitation, distinguishing a symptom complex from other types of adverse reactions may be potentially useful for risk mitigation. In another embodiment, and without limitation, a mechanism of action pertaining to therapeutic oligomer 156 may include cross-linking of cell surface-expressed receptors, which may be the targets of the therapeutic protein product (e.g., CD28 expressed on T-cells). In another embodiment, and without limitation, a risk-based evaluation on the mechanism of action of a therapeutic protein product as well as results of animal and/or in vitro evaluations may be performed to determine the need for a collection of pre- and post-dose cytokine levels in an early phase of clinical development. In another embodiment, and without limitation, an evaluation may provide evidence to support a clinical diagnosis of cytokine release syndrome and help distinguish it from other acute drug reactions.

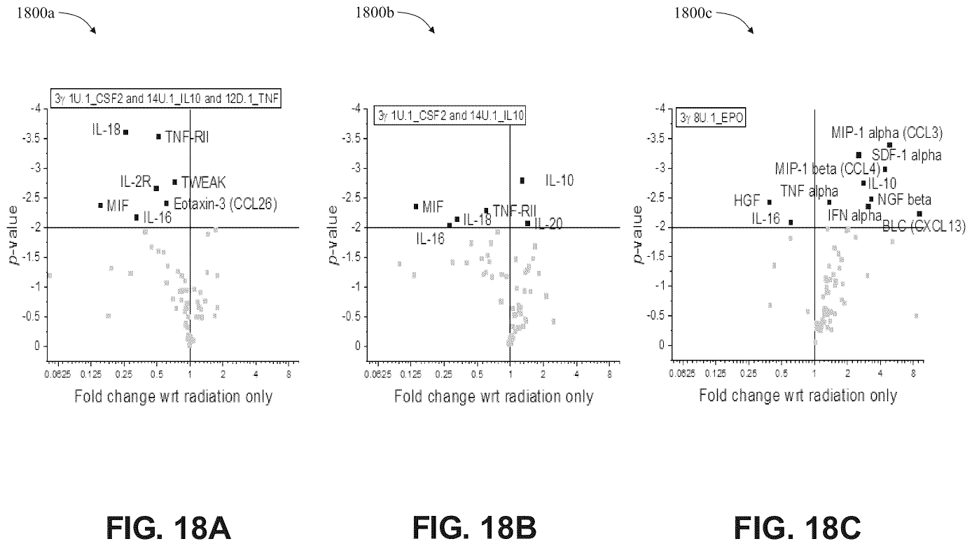

Now referring to FIGS. 18A-C, exemplary embodiments 1800a-c of an efficacy of therapeutic oligomer 156 are illustrated. Referring now to FIG. 18A, therapeutic oligomer 156 may include an oligomer designed to upregulate GM-CSF and/or CSF2, which may result in a significant increase in protein expression. Such increase may be measured using cytokine quantification on PBMCs stimulated using a 3-Gy gamma radiation. Expressed protein may include a GM-CSF, an associated G-CSF and other growth factors, proinflammatory cytokines (IL-1 a, IL-1 b, TNF-α, TNF receptors, etc.), and/or IL-10 included in a gene interaction network. Referring now to FIG. 18B, therapeutic oligomer 156 may include an oligomer designed to upregulate GM-CSF and/or CSF2, which may result in a significant increase in protein expression, wherein the increase may be measured using cytokine quantification on PBMCs stimulated using a 3-Gy gamma radiation. Expressed protein may include a GM-CSF, an associated G-CSF and other growth factors, proinflammatory cytokines (IL-1 α, IL-1 b, TNF-α, TNF receptors, etc.), and/or IL-10 included in a gene interaction network. Referring now to FIG. 18C, therapeutic oligomer 156 may upregulate hemopoietic proteins and/or proinflammatory enzymes. In an embodiment, and without limitation, therapeutic oligomer 156 may reduce the probability of developing cancer as a function of upregulating hemopoietic proteins and/or associated growth factors.

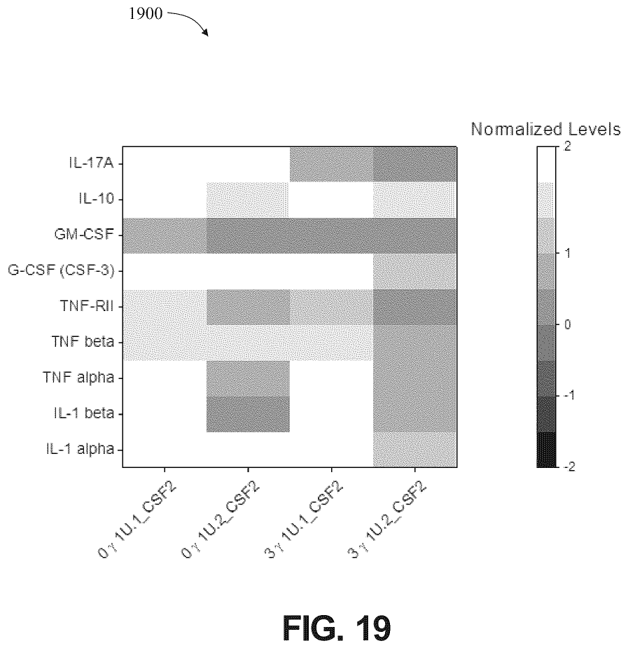

Now referring to FIG. 19, an exemplary embodiment 1900 of a therapeutic effect of a therapeutic nanoligomer is illustrated. In an embodiment, and without limitation, therapeutic nanoligomer may have a therapeutic effect of upregulating a growth factor and/or hemopoietic protein. Such upregulation may result in a significant increase in protein expression of targeted growth factor proinflammatory cytokines, such as but not limited to IL-1 α, IL-1 B, TNF-α, TNF receptors, and/or IL-10, among others. Such therapeutic effect may be characterized via cytokine quantification on PBMCs stimulated using 3-Gy gamma radiation.

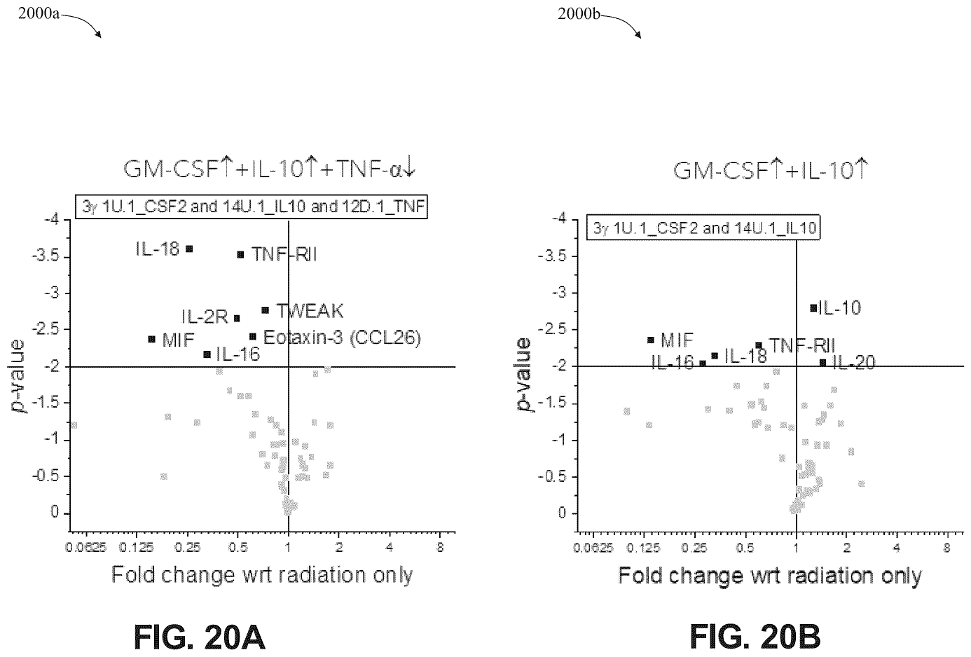

Now referring to FIGS. 20A-B, exemplary embodiments 2000a-b of a therapeutic effect of therapeutic oligomer 156 are illustrated. Referring now to FIG. 20A, therapeutic oligomer 156 may be multiplexed using 10 mM CSF-2 upregulators, IL-10 upregulators, and/or TNF-α downregulators, wherein the multiplexed therapeutic oligomer 156 may exert a therapeutic effect on one or more cytokine expression profiles. Referring now to FIG. 20B, therapeutic oligomer may be multiplied using 10 mM CSF-2 upregulators and/or IL-10 upregulators, wherein the multiplexed therapeutic oligomer 156 may reduce the increase in expression of proinflammatory cytokines and/or result in an increase in IL-10 expression. In an embodiment, and without limitation, multiplexing therapeutic oligomers 156 may allow for precise tuning of the therapeutic profile of immune engineering therapeutics.

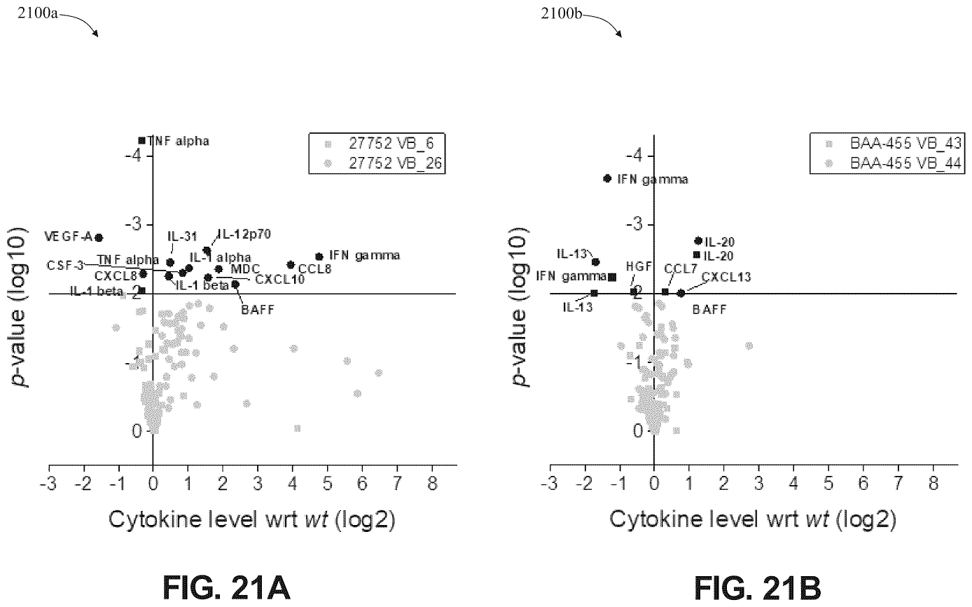

Now referring to FIGS. 21A-B, exemplary embodiments 2100a-b of a genomic outcome are illustrated. In an embodiment, and without limitation, therapeutic oligomer 156 may modulate one or more gene expressions in a range of genetically intractable anaerobes to tweak the human microbiome for a plurality of health and/or wellness purposes. Referring now to FIG. 21A, Pseudobutyrivibrio xylanivorans may be cultured, and two genes of interest may be identified from patients undergoing clinical trials for an immune checkpoint therapy: 1) Gene 1: alpha/beta hydrolase family protein and 2) Gene 2: helix-turn-helix domain-containing protein. In an embodiment, and without limitation, an observed effect may include a strong downregulation of pro-inflammatory cytokines and/or upregulation of anti-inflammatory cytokines upon an application of their antisense therapeutic oligomers 156, followed by treating anaerobic cultures, collecting cell lysates, and/or treating human PBMCs. In another embodiment, and without limitation, bacterial lysates may be used to isolate bacterial metabolites, wherein the isolated bacterial metabolites may be used for metabolic sequencing. Therapeutic oligomers 156 may be developed for oral delivery in an enteric polymer coating to treat autoimmune diseases. Referring now to FIG. 21B, two or more genes, such as but not limited to Gene 1: metallo-hydrolase-like_MBL-fold and/or Gene 2: glf UDP-galactopyranose mutase in Blautia hansenii, may be targeted to increase the expression of proinflammatory cytokines, wherein an exposure of bacterial lysates to human PBMCs may occur. In an embodiment, and without limitation, bacterial metabolites and/or therapeutic oligomers 156 may include validated targets for the development of anticancer drugs by modulating the human microbiome.

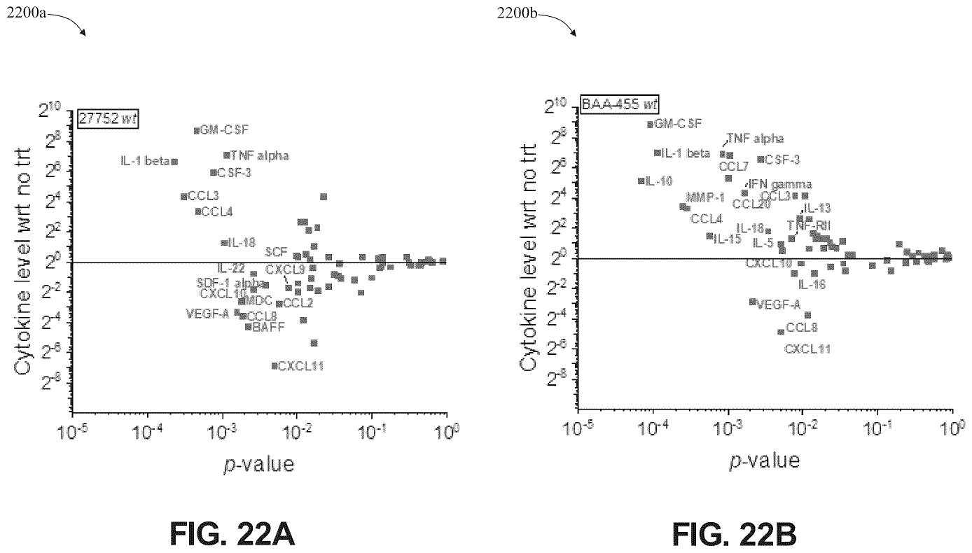

Now referring to FIGS. 22A-B, exemplary embodiments 2200a-b of a therapeutic effect of therapeutic oligomer 156 is illustrated. In an embodiment, and without limitation, therapeutic effect may be an effect of target microbes on human cytokine expression. Referring now to FIG. 22A, bacterial lysates of Pseudobutyrivibrio xylanivorans (ATC27752), may be evaluated such that an impact on cytokine expression to develop bacterial metabolites and growth-promoting/inhibiting therapeutic oligomers to perturb the human microbiome may be determined. Referring now to FIG. 22B, bacterial lysates of Blautia hansenii (BAA-455) may be evaluated such that an impact on cytokine expression to develop bacterial metabolites and growth-promoting/inhibiting therapeutic oligomers 156 to perturb the human microbiome may be determined. In an embodiment, and without limitation, Pseudobutyrivibrio xylanivorans (ATCC 27752) and/or Blautia hansenii (BAA-455) may both promote growth factors, such as but not limited to GM-CSF, G-CSF and/or pro-inflammatory cytokine expression, which may result in a desired drug profile for promoting growth while ensuring the elimination of cancer development. In an embodiment, and without limitation, any undesired change in cytokines or other protein expressions may be easily targeted/eliminated, and/or an expression of desired proteins may be further increased, as a function of addition of desired therapeutic oligomer 156.

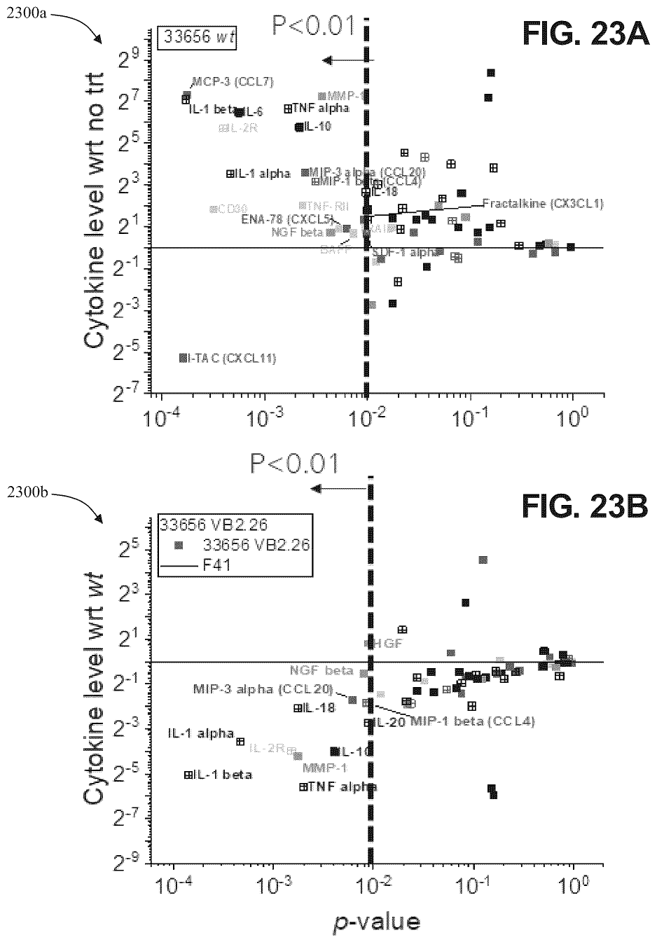

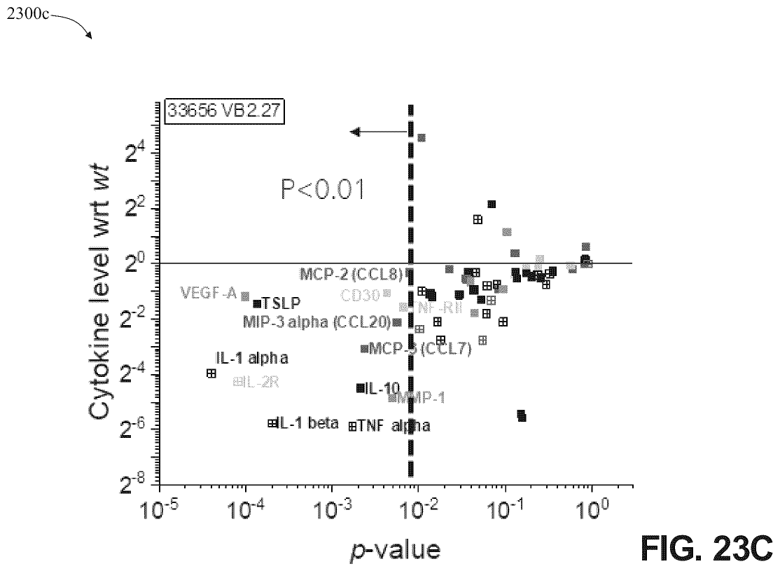

Now referring to FIG. 23A-C, exemplary embodiments 2300a-c of a therapeutic effect of therapeutic oligomer 156 is illustrated. In an embodiment, and without limitation, therapeutic effect of therapeutic oligomer 156 may include a therapeutic effect on a human microbiome. Referring now to FIG. 23A, therapeutic effect may include an effect of Eubacterium rectale bacterial lysates on a human microbiome, wherein the therapeutic effect may lead to a strong increase in pro-inflammatory cytokine expression in human PBMCs. Referring now to FIG. 23B, an addition of antisense therapeutic oligomer 156 may target a scfB thioether cross-link-forming SCIFF peptide maturase gene in Eubacterium rectale, wherein the addition may lead to increase in anti-inflammatory cytokine and/or reduction in pro-inflammatory cytokine production. Referring now to FIG. 23C, an addition antisense therapeutic oligomer 156 may result in a therapeutic effect of targeting peptidase domain-containing ABC transporter gene in Eubacterium rectale, wherein the therapeutic effect may lead to a strong anti-inflammatory cytokine expression. In an embodiment, and without limitation, all three bacterial metabolites and two therapeutic oligomers may be developed as health and wellness products. In another embodiment, and without limitation, an anti-inflammatory response may be used for targeting autoimmune diseases, such as but not limited to Crohn's and colitis, inflammatory bowel disease, and/or for targeting proinflammatory cytokines for anticancer drugs using the human microbiome, consistent with details described above in this disclosure.

Referring now to FIG. 24, an exemplary embodiment of a naive Bayes classification model 2400 that may perform one or more machine-learning processes as described in this disclosure is illustrated. Naïve Bayes classification model 2400 may be configured to classify inputs 2404 into a plurality of Bayes categories. A naïve Bayes classification algorithm may generate classifiers by assigning Bayes categories to inputs 2404. In some embodiments, this may be done using a Bayes category classifier 2408. As used in the current disclosure, a “Bayes category classifier” is a machine-learning model that sorts inputs into categories or bins of data, outputting the categories or bins of data and/or labels associated therewith. Bayes category classifier 2408 receives input 2404 and sorts the input into a plurality of Bayes categories and Bayes vectors. As used in the current disclosure, a “Bayes category” is a set of categories specific to a given input. Bayes categories may include both categories and subcategories, wherein the categories may include possible outcomes for naive Bayes classification model 2400 and the subcategories may include variables that are outcome determinative for the classification model 2400. Bayes categories and subcategories may be drawn from a finite set of variables within input 2404. Once inputs 2404 are classified into Bayes categories and subcategories, they may be represented as Bayes vectors. In some embodiments, Bayes categories and subcategories may be reflected as a histogram or a frequency table. As used in the current disclosure, a “Bayes vector” is an enumeration of each input within a given Bayes category. As a nonlimiting example, a Bayes vector may be the number of times a given element occurs in a Bayes subcategory.

Still Referring to FIG. 24, “training data”, as used herein, is data containing correlations that a machine-learning process may use to model relationships between two or more categories of data elements. For instance, and without limitation, training data 2412 may include a plurality of data entries, each entry representing a set of data elements that were recorded, received, and/or generated together; data elements may be correlated by shared existence in a given data entry, by proximity in a given data entry, or the like. In some embodiments, training data 2412 may be the same or substantially similar to the training data disclosed within FIG. 7. Training data 2412 may include identification of Bayes categories and subcategories. Training data 2412 may additionally include outcomes of previous naive Bayes classification model 2400.

Still Referring to FIG. 24, a naive Bayes classification model 2400 may apply Bayes theorem 2416 as a function of the output of Bayes category classifier 2408. As used in the current disclosure, “Bayes theorem” is a theorem describing the probability of an event based on prior knowledge of conditions that might be related to the event. In some embodiments, Bayes theorem 2416 may be expressed as P(A/B)=P(B/A)×P(A)=P(B), where P(A/B) is the probability of hypothesis A given data B, also known as posterior probability; P(B/A) is the probability of data B given that the hypothesis A was true; P(A) is the probability of hypothesis A being true regardless of data, also known as prior probability of A; and P(B) is the probability of the data regardless of the hypothesis. In some embodiments, applying Bayes theorem 2416 may include replacing variables with Bayes vectors and Bayes categories/subcategories.

Still Referring to FIG. 24, a naive Bayes classification model 2400 may be configured to generate a naive Bayes classifier 2420 as a function of Bayes theorem 2416. As used in the current disclosure, “Naïve Bayes classifiers” are a family of probabilistic classifiers based on applying Bayes' theorem 2416 with strong (naive) independence assumptions between the features. In an embodiment, naive Bayes classifier 2420 may include a classifier or a machine-learning model as defined herein above in FIG. 7. Generating naive Bayes classifier 2420 may include generating a family of algorithms that assume that the value of a particular element is independent of the value of any other element, given a class variable. A naïve Bayes classifier 2420 may include a multinomial naive Bayes classifier or a Gaussian naive Bayes classifier. Naïve Bayes classification module may be implemented as any hardware and/or software module. Naïve Bayes classification module may be configured to generate a naive Bayes classification algorithm. A naïve Bayes classifier 2420 may additionally be trained using training data 2412.

Still referring to FIG. 24, naive Bayes classifier 2420 may be configured to output 2424 a posterior probability as a function input 2404. Bayes categories and subcategories may utilize Bayes theorem 2416 to calculate a posterior probability for each class. As used in the current disclosure, a “posterior probability” is a type of conditional probability that results from updating the prior probability with information summarized by the likelihood, through an application of Bayes theorem 2416. A posterior probability distribution may describe the epistemic uncertainty about statistical parameters conditional on a collection of observed data. From a given posterior distribution, various point and interval estimates can be derived, such as the maximum a posteriori (MAP) or the highest posterior density interval (HPDI). A class containing the highest posterior probability is the outcome of prediction. Naïve Bayes classification algorithm may include a gaussian model that follows a normal distribution. Naive Bayes classification algorithm may include a multinomial model that is used for discrete counts. Naive Bayes classification algorithm may include a Bernoulli model that may be utilized when vectors are binary.

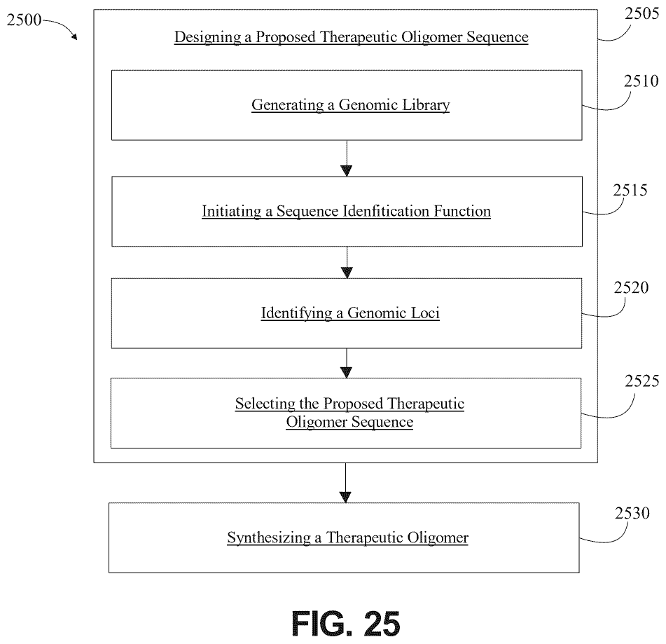

Now referring to FIG. 25, a method for producing therapeutic oligomer 156 is illustrated. At step 2505, a computing device 104 designs a proposed therapeutic oligomer sequence 108. Computing device 104 includes any of computing device 104 as described above, in reference to FIGS. 1-24. Therapeutic oligomer sequence 108 includes any of the therapeutic oligomer sequence 108 as described above, in reference to FIGS. 1-24.

Still referring to FIG. 25, at step 2510, computing device 104 generates a genomic library 112 for an organism. Genomic library 112 may include any of the genomic library 112 as described above, in reference to FIGS. 1-24. Computing device 104 generates genomic library 112 for an organism from gene target 116. Gene target 116 may include any of the gene target 116 as described above, in reference to FIGS. 1-24.

Still referring to FIG. 25, at step 2515, computing device 104 initiates a sequence identification function 120. Sequence identification function 120 may include any of the sequence identification function 120 as described above, in reference to FIGS. 1-24. Computing device 104 initiates sequence identification function 120 as a function of identifying a plurality of prospective gene targets 124 and genomic library 112. Prospective gene target 116 may include any of the prospective gene target 124 as described above, in reference to FIGS. 1-24. Computing device 104 initiates sequence identification function 120 as a function of the plurality of prospective gene targets 124 and an oligomeric machine-learning model 128. Oligomeric machine-learning model 128 may include any of the oligomeric machine-learning model 128 as described above, in reference to FIGS. 1-24. Oligomeric machine-learning model 128 is trained as a function of an oligomeric training set. Oligomeric training set includes any of the oligomeric training set as described above, in reference to FIGS. 1-24. Oligomeric training set correlates the plurality of prospective gene targets to an oligomer that regulates a gene expression. Oligomer that regulates gene expression may include any of the oligomer that regulates gene expression as described above, in reference to FIGS. 1-24.

Still referring to FIG. 25, at step 2520, computing device 104 identifies a genomic locus 132 that proposed therapeutic oligomer sequence 108 is predicted to bond to as a function of an off-target sequence function 136. Genomic locus 132 may include any of the genomic locus 132 as described above, in reference to FIGS. 1-24. Off-target sequence function 136 may include any of the off-target sequence function 136 as described above, in reference to FIGS. 1-24. Computing device 104 identifies an incidental alignment 140 as a function of proposed therapeutic oligomer sequence 108 and genomic library 112. Incidental alignment 140 may include any of the incidental alignment 140 as described above, in reference to FIGS. 1-24. Computing device 104 identifies genomic locus 132 as a function of modeling incidental alignment 140 to a corresponding genome assembly location 144. Genome assembly location 144 may include any of the genome assembly location 144 as described above, in reference to FIGS. 1-24. Computing device 104 identifies genomic locus 132 as a function of incidental alignment 140 and an overlap element 148. Overlap element 148 may include any of the overlap element 148 as described above, in reference to FIGS. 1-24.

Still referring to FIG. 25, at step 2525, computing device 104 selects proposed therapeutic oligomer sequence 108 as a function of sequence identification function 120, genomic locus 132, and criterion element 152. Criterion element 152 may include any of the criterion element 152 as described above, in reference to FIGS. 1-24.

Still referring to FIG. 25, at step 2530, computing device 104 synthesizes a therapeutic oligomer 156 as a function of proposed therapeutic oligomer sequence 108. Therapeutic oligomer 156 may include any of the therapeutic oligomer 156 as described above, in reference to FIGS. 1-24. In an embodiment, and without limitation, computing device 104 may synthesize therapeutic oligomer 156 using automated synthesizer 160. Automated synthesizer 160 may include any of the automated synthesizer 160 as described above, in reference to FIGS. 1-24. In an embodiment, and without limitation, computing device 104 may update genomic library 112 as a function of therapeutic oligomer 156.

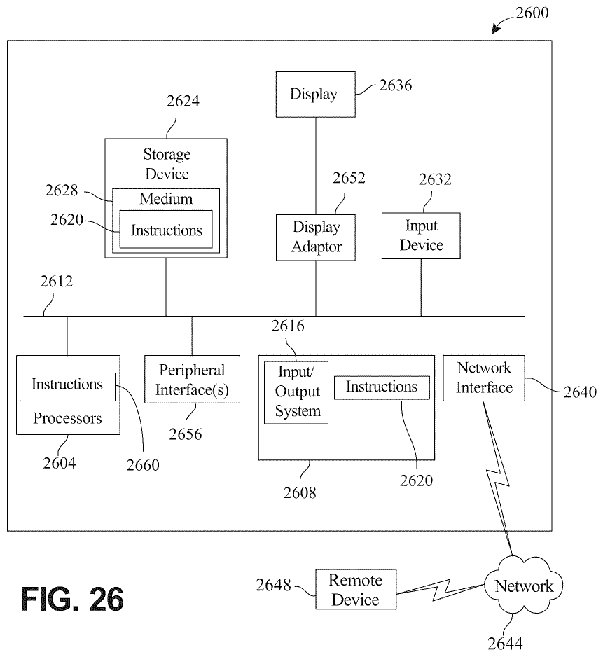

Referring now to FIG. 26, it is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to one of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module. Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission. Such software may also include information (e.g., data) carried as a data signal on a data carrier, such as a carrier wave. For example, machine-executable information may be included as a data-carrying signal embodied in a data carrier in which the signal encodes a sequence of instructions, or portion thereof, for execution by a machine (e.g., a computing device) and any related information (e.g., data structures and data) that causes the machine to perform any one of the methodologies and/or embodiments described herein. Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

With continued reference to FIG. 26, the figure shows a diagrammatic representation of one embodiment of a computing device in the exemplary form of a computing system 2600 within which a set of instructions for causing the computing system 2600 to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computing system 2600 may include a processor 2604 and a memory 2608 that communicate with each other, and with other components, via a bus 2612. Bus 2612 may include any of several types of bus structures including, but not limited to, a memory bus, a memory controller, a peripheral bus, a local bus, and any combinations thereof, using any of a variety of bus architectures. Processor 2604 may include any suitable processor, such as without limitation a processor incorporating logical circuitry for performing arithmetic and logical operations, such as an arithmetic and logic unit, which may be regulated with a state machine and directed by operational inputs from memory and/or sensors; processor 2604 may be organized according to Von Neumann and/or Harvard architecture as a non-limiting example. Processor 2604 may include, incorporate, and/or be incorporated in, without limitation, a microcontroller, microprocessor, digital signal processor, field programmable gate array, complex programmable logic device, graphical processing unit, general-purpose graphical processing unit, tensor processing unit, analog or mixed signal processor, trusted platform module, a floating-point unit, and/or system on a chip.

With continued reference to FIG. 26, memory 2608 may include various components (e.g., machine-readable media) including, but not limited to, a random-access memory component, a read-only component, and any combinations thereof. In one example, a basic input/output system 2616, including basic routines that help to transfer information between elements within computing system 2600, such as during start-up, may be stored in memory 2608. Memory 2608 (e.g., stored on one or more machine-readable media) may also include instructions (e.g., software) 2620 embodying any one or more of the aspects and/or methodologies of the present disclosure. In another example, memory 2608 may further include any number of program modules including, but not limited to, an operating system, one or more application programs, other program modules, program data, and any combinations thereof.

With continued reference to FIG. 26, computing system 2600 may also include a storage device 2624. Examples of a storage device (e.g., storage device 2624) include, but are not limited to, a hard disk drive, a magnetic disk drive, an optical disc drive in combination with an optical medium, a solid-state memory device, and any combinations thereof. Storage device 2624 may be connected to bus 2612 by an appropriate interface (not shown). Example interfaces include, but are not limited to, small computer system interface, advanced technology attachment, serial advanced technology attachment, universal serial bus, IEEE 1394 (FIREWIRE), and any combinations thereof. In one example, storage device 2624 (or one or more components thereof) may be removably interfaced with computing system 2600 (e.g., via an external port connector (not shown)). Particularly, storage device 2624 and an associated machine-readable medium 2628 may provide nonvolatile and/or volatile storage of machine-readable instructions, data structures, program modules, and/or other data for computing system 2600. In one example, software 2620 may reside, completely or partially, within machine-readable medium 2628. In another example, software 2620 may reside, completely or partially, within processor 2604.

With continued reference to FIG. 26, computing system 2600 may also include an input device 2632. In one example, a user of computing system 2600 may enter commands and/or other information into computing system 2600 via input device 2632. Examples of input device 2632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 2632 may be interfaced to bus 2612 via any of a variety of interfaces (not shown) including, but not limited to, a serial interface, a parallel interface, a game port, a USB interface, a FIREWIRE interface, a direct interface to bus 2612, and any combinations thereof. Input device 2632 may include a touch screen interface that may be a part of or separate from display device 2636, discussed further below. Input device 2632 may be utilized as a user selection device for selecting one or more graphical representations in a graphical interface as described above.

With continued reference to FIG. 26, user may also input commands and/or other information to computing system 2600 via storage device 2624 (e.g., a removable disk drive, a flash drive, etc.) and/or network interface device 2640. A network interface device, such as network interface device 2640, may be utilized for connecting computing system 2600 to one or more of a variety of networks, such as network 2644, and one or more remote devices 2648 connected thereto. Examples of a network interface device include, but are not limited to, a network interface card (e.g., a mobile network interface card, a LAN card), a modem, and any combination thereof. Examples of a network include, but are not limited to, a wide-area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 2644, may employ a wired and/or a wireless mode of communication. In general, any network topology may be used. Information (e.g., data, software 2620, etc.) may be communicated to and/or from computing system 2600 via network interface device 2640.

With continued reference to FIG. 26, computing system 2600 may further include a video display adapter 2652 for communicating a displayable image to a display device, such as display device 2636. Examples of a display device include, but are not limited to, a liquid crystal display (LCD), a cathode ray tube (CRT), a plasma display, a light emitting diode (LED) display, and any combinations thereof. Display adapter 2652 and display device 2636 may be utilized in combination with processor 2604 to provide graphical representations of aspects of the present disclosure. In addition to a display device, computing system 2600 may include one or more other peripheral output devices including, but not limited to, an audio speaker, a printer, and any combinations thereof. Such peripheral output devices may be connected to bus 2612 via a peripheral interface 2656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A therapeutic nanoligomer composition comprising:
at least a peptide nucleic acid (PNA), the at least a PNA comprising:
a sequence of nucleobases capable of interacting with a gene target in a target host; and
a polypeptide backbone attached to the sequence of nucleobases, the polypeptide backbone comprising a plurality of amino acid units, wherein the plurality of amino acid units comprises at least a 2-N-aminoethylglycine unit; and
a transport domain, the transport domain comprising:
a delivery nanoparticle, a cellular uptake domain (CUD) associated with the delivery nanoparticle, wherein the delivery nanoparticle is attached to the at least a PNA, and wherein the CUD comprises hydrophilic polymeric coating or one or more weakly charged surface groups.

2. The therapeutic nanoligomer composition of claim 1, wherein the least a PNA further comprises a nucleic acid-binding domain.

3. The therapeutic nanoligomer composition of claim 1, wherein the sequence of nucleobases comprise sequences selected from a group consisting of SEQ ID NO: 27-28.

4. The therapeutic nanoligomer composition of claim 1, wherein the CUD comprises cysteine, glutathione, or a combination thereof.

5. The therapeutic nanoligomer composition of claim 4, wherein the glutathione comprises eighteen glutathione molecules configured to enable active transport across a blood-brain barrier.

6. The therapeutic nanoligomer composition of claim 1, wherein the hydrophilic polymeric coating comprises poly(ethylene glycol), poly(ethylene oxide), poly(vinyl alcohol), poly(vinylpyrrolidone), poly(2-oxazoline), polysaccharides, zwitterionic polymers, poly(acrylamide), poly(amino acid) polymers, or a combination thereof.

7. The therapeutic nanoligomer composition of claim 1, wherein the one or more weakly charged surface groups comprise primary, secondary, or tertiary amines, protonatable heterocycles, carboxylate, phosphate, or sulfonate groups, zwitterionic moieties, pH-responsive functional groups or a combination thereof.

8. The therapeutic nanoligomer composition of claim 1, wherein:
the at least a PNA further comprises a nanoparticle binding element, wherein the nanoparticle binding element comprises a linker, wherein the linker comprises a sequence selected from a group consisting of SEQ ID NO: 1-2; and
the delivery nanoparticle is attached to the at least a PNA through the nanoparticle binding element.

9. The therapeutic nanoligomer composition of claim 1, wherein the at least a PNA further comprises a nuclear localization sequence (NLS) selected from a group consisting of SEQ ID NO: 3-7.

10. The therapeutic nanoligomer composition of claim 1, wherein the at least a PNA further comprises a transcriptional activation domain.

11. The therapeutic nanoligomer composition of claim 10, wherein the transcriptional activation domain comprises one or more acidic domains selected from a group consisting of SEQ ID NO: 12-24.

12. The therapeutic nanoligomer composition of claim 1, wherein the delivery nanoparticle comprises a transition-metal nanoparticle.

13. The therapeutic nanoligomer composition of claim 12, wherein the transition-metal nanoparticle comprises a gold nanoparticle, wherein the gold nanoparticle comprises Au22 or Au25 nanoparticles.

14. The therapeutic nanoligomer composition of claim 12, wherein the at least a PNA further comprises a linker, wherein the linker is configured to link the at least a PNA and the transition-metal nanoparticle.

15. The therapeutic nanoligomer composition of claim 1, wherein the at least a PNA further comprises:
a nucleic acid-binding domain; and
a nanoparticle binding element, wherein the nanoparticle binding element comprises a linker, wherein the linker is configured to link the nucleic acid-binding domain and the nanoparticle binding element.

16. The therapeutic nanoligomer composition of claim 1, further comprising a pharmaceutically acceptable excipient.

17. The therapeutic nanoligomer composition of claim 16, wherein the pharmaceutically acceptable excipient comprises solvents, dispersion media, coatings, antibacterial and antifungal agents, isotonic and absorption delaying agents, liquid or solid fillers, diluents, excipients, manufacturing aids, and solvent-encapsulating materials.

18. The therapeutic nanoligomer composition of claim 1, wherein the therapeutic nanoligomer composition is administered to a subject via one or more manners selected from a group consisting of oral administration, nasal administration, intraperitoneal administration, parenteral administration, intravenous administration, intramuscular administration, topical administration, subcutaneous administration, and injection into tissue.

19. The therapeutic nanoligomer composition of claim 1, wherein the gene target is associated with one or more members selected from a group consisting of chromosome, plasmid, DNA, RNA, dsRNA, dsDNA, mRNA, siRNA, RNA, hpRNA, RNA sequencing target, Interleukin-1 including Interleukin-1α(IL-1α) and Interleukin-1β (IL-1β), tumor necrosis factor alpha (TNF-α), tumor necrosis factor-alpha receptor 1 (TNF-R1), Interleukin-4 (IL-4), Interleukin-6 (IL-6), Interleukin-10 (IL-10), Interleukin-13 (IL-13), Interleukin-18 (IL-18), Interleukin-31 (IL-31), inflammasomes including NOD-like receptor family, pyrin domain containing 1 (NLRP1), NOD-like receptor family, pyrin domain containing 3 (NLRP3), NOD-like receptor family, pyrin domain containing 4 (NLRP4), NOD-like receptor family, pyrin domain containing 6 (NLRP6), Interferon-inducible protein AIM2 (AIM2), granulocyte-macrophage colony-stimulating factor (GM-CSF), Granulocyte colony-stimulating factor (G-CSF), key transcription factor including nuclear factor kappa-light-chain-enhancer of activated B cells (NF-κB), erythropoietin (EPO), gamma globulin (GG), TAR DNA binding protein 43 (TDP-43), telomerase reverse transcriptase (TERT), chemokine ligands 4 (CCL4), chemokine ligands 20 (CCL20), C—X—C motif chemokine 5 (CXCL5), C—X—C motif chemokine 11 (CXCL11), CD40 ligand (CD40L), TNF receptor superfamily member 8 (CD30), and microRNA including miR-2392.

20. The therapeutic nanoligomer composition of claim 1, wherein the at least a PNA is configured to upregulate a gene expression in a target host.

21. The therapeutic nanoligomer composition of claim 20, wherein the gene expression in the target host comprises GM-CSF or CSF2.

22. The therapeutic nanoligomer composition of claim 1, wherein the at least a PNA is configured to downregulate a gene expression in a target host.

23. The therapeutic nanoligomer composition of claim 20, wherein the gene expression in the target host comprises Interleukin-1 (IL-1) such as Interleukin-1α(IL-1α) and Interleukin-1β (IL-1β), tumor necrosis factor alpha (TNF-α), tumor necrosis factor-alpha receptor 1 (TNF-R1), Interleukin-4 (IL-4), Interleukin-6 (IL-6), Interleukin-10 (IL-10), Interleukin-13 (IL-13), Interleukin-18 (IL-18), or Interleukin-31 (IL-31).

24. The therapeutic nanoligomer composition of claim 1, wherein the at least a PNA comprises:
a first PNA configured to interact with a first gene target comprising NLRP3; and
a second PNA configured to interact with a second gene target comprising NF-κB,
wherein the first PNA and the second PNA are attached to the transport domain.

25. The therapeutic nanoligomer composition of claim 1, wherein the delivery nanoparticle has a total hydrodynamic size of less than 2 nm.

26. The therapeutic nanoligomer composition of claim 1, wherein the delivery nanoparticle has a total hydrodynamic size of 4 nm to 5 nm.
